(19) United States
(12) Patent Application Publication
(10) Pub. No.: US 2023/0090742 A1
(43) Pub. Date: Mar. 23, 2023

(54) AMINOPYRIMIDINYLAMINOBENZONITRILE DERIVATIVES AS NEK2 INHIBITORS

(71) Applicant: Sumitomo Pharma Oncology, Inc., Cambridge, MA (US)

(72) Inventors: Adam Siddiqui-Jain, South Jordan, UT (US); Steven L. Warner, Cambridge, UT (US); Harshil Dhruv, Cambridge, UT (US)

(21) Appl. No.: 17/759,147

(22) PCT Filed: Jan. 29, 2021
(86) PCT No.: PCT/US2021/015758

§ 371 Date: Jul. 20, 2022

Related U.S. Application Data
(60) Provisional application No. 62/968,033, filed on Jan. 30, 2020.

(51) Int. Cl. A61K 31/506; C07D 239/48; C07D 401/14; C07D 403/14; C07D 403/12; A61K 31/5377

(52) U.S. Cl. CPC A61K 31/506; C07D 239/48; C07D 401/14; C07D 403/14; C07D 403/12; A61K 31/5377

(57) ABSTRACT

The present invention provides aminopyrimidinylaminobenzonitrile compounds that inhibit the activity of never in mitosis gene A-related kinase 2 (NEK2) and are useful in the treatment of diseases related to activity of NEK2, including cancer (e.g., multiple myeloma, and breast, liver, pancreatic and colorectal cancers).

### RELATED APPLICATIONS

This application claims the benefit of U.S. Provisional Application No. 62/968,033, filed Jan. 30, 2020. The entire disclosure of this application is incorporated herein by reference.

### FIELD OF THE INVENTION

The present invention provides aminopyrimidinylaminobenzonitrile compounds that inhibit the activity of never in mitosis gene A-related kinase 2 (NEK2) and are useful in the provision of treatment of diseases related to activity of NEK2, including cancer (e.g., multiple myeloma, breast, liver, pancreatic and colorectal cancer).

### BACKGROUND OF THE INVENTION

Cancer is characterized as the uncontrolled proliferation of cells in the body due to the abnormal activity of various proteins. (Kokuryo, T. et al. Anticancer Research 2019, 39, 2251-2258). It has been suggested that cell cycle proteins play a key role in multiple cancer types. Many forms of cancer are dependent on cell cycle-regulated proteins and tend to be sensitive to their inhibition (Xia, J. et al. BioMed Research International 2015, 1-12; Fang, Y. et al. Cell Cycle 2016, 15(7), 895-907). As such, cell cycle regulators are sought out as targets for cancer treatment.

Never in mitosis gene A-related kinase 2 (NEK2) is a serine/threonine protein kinase, belonging to the NEK family of cell cycle regulator proteins (Kokuryo, T. et al., NEK2 Is an Effective Target for Cancer Therapy With Potential to Induce Regression of Multiple Human Malignancies, Anticancer Research 2019, 39, 2251-2258, doi/10.21873/anticanres.13341; Meng, L. et al. BioMed Research International 2014, 1-13; Fang, Y. et al. Cell Cycle 2016, 15(7), 895-907; Xia, J. et al., Role of NEK2A in Human Cancer and Its Therapeutic Potentials, BioMed Research International 2015, 1-12, doi/10.1155/2015/862461). The first member of this family, the mitotic regulator never in mitosis gene A (NIMA), was originally identified as a mutant preventing Aspergillus nidulans cells from entering mitosis. The NEK family has eleven members (NEK1 to NEK11), with NEK2 having the highest sequence identity to NIMA (Meng, L. et al. BioMed Research International 2014, 1-13; Kokuryo, T. et al. Anticancer Research 2019, 39, 2251-2258). NEK2 in mammals is expressed as three slice variants: NEK2A, NEK2B and NEK2C. NEK2A is the full length protein with 445 amino acids (48 KDa). It has structures with an N-terminal catalytic kinase domain and a C-terminal catalytic domain. The C-terminus has multiple regulatory motifs, including a leucine zipper, coiled coil, centrosome and microtubule localization sites, protein phosphatase 1 (PP1) binding site, KEN-box, nucleolar localization sites, anaphase-promoting complex (APC) binding site, and destruction-box (Fang, Y. et al. Cell Cycle 2016, 15(7), 895-907; Kokuryo, T. et al. Anticancer Research 2019, 39, 2251-2258). There is substantial evidence that NEK2 plays a central role in centrosome separation and promotion of the cell cycle from G₂ to M phase. NEK2 overexpression results in chromosome instability and aneuploidy in cancer cells. NEK2 overexpression also activates several oncogenic pathways and ATP-binding cassette transporters, thereby leading to cell proliferation, invasion and drug resistance (Zhou, W. et al. Cancer Cell 2013, 23(1), 48-62; Kokuryo, T. et al. Anticancer Research 2019, 39, 2251-2258).

It has been suggested that inhibition of NEK2 can be beneficial in providing treatment for patients with cancer. NEK2 is highly expressed in multiple cancer types, for example, but not limited to, Ewing's sarcoma, breast, colorectal, testicular, cervical, liver, prostate, lung, ovarian, renal cell cancer, myeloma, lymphoma, pancreatic, cholangiocarcinoma and others (Kokuryo, T. et al. Anticancer Research 2019, 39, 2251-2258; Wu, S. et al. International Journal of Cancer 2016, 140, 1581-1596; Zhou, W. et al. Cancer Cell 2013, 23(1), 48-62). NEK2 overexpression is significantly associated with histological differentiation, higher “TNM” classification of malignant tumor stage, lymph node metastasis, and tumor invasion in colon, pancreatic, and lung cancer. NEK2 is a promising predictor of poor prognosis in cancer because its expression is highly correlated with rapid relapse and poor outcome in multiple cancer types (Fang, Y. et al. Cell Cycle 2016, 15(7), 895-907; Kokuryo, T. et al. Anticancer Research 2019, 39, 2251-2258).

Inhibitors of NEK2 are widely sought and several classes of compounds have been reported, for example: Meng, L. et al. BioMed Research International 2014, 1-13; Fang, Y. et al. Cell Cycle 2016, 15(7), 895-907; and Kokuryo, T. et al. Anticancer Research 2019, 39, 2251-2258.

Thus, new or improved agents which inhibit NEK2 are continually needed that act as agents for the prevention and treatment of diseases, including cancer (e.g., but not limited to, multiple myeloma, breast, liver, pancreatic and colorectal cancer). The compounds, compositions and methods described herein are directed towards these and other ends.

### SUMMARY OF THE INVENTION

The present invention provides compounds of Formula I:

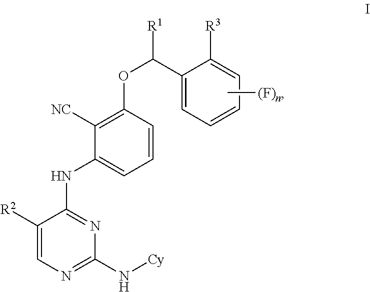

or a pharmaceutically acceptable salt thereof, wherein R¹, R², R³, n, and Cy are defined hereinbelow.

The present invention further provides compounds of Formula II:

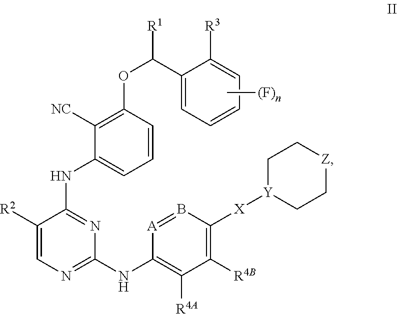

or a pharmaceutically acceptable salt thereof, wherein R¹, R², R³, n, R^{4A}, R^{4B}, A, B, X, Y, and Z are defined hereinbelow.

The present invention further provides compounds of Formula III:

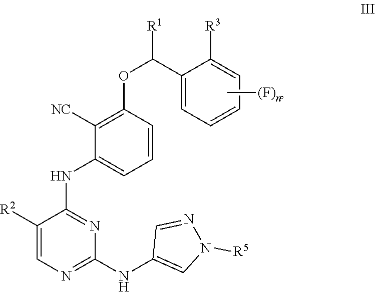

or a pharmaceutically acceptable salt thereof, wherein R¹, R², R³, n, and R⁵ are defined hereinbelow.

The present invention further provides compositions comprising at least one compound selected from compounds of Formula I, II, or III, or from any of the compounds of the invention exemplified herein, or one or more of any of these in the form of a pharmaceutically acceptable salt. In some embodiments the composition comprises at least one pharmaceutically acceptable excipient which may be part of a pharmaceutically acceptable carrier.

The present invention further provides methods of inhibiting the activity of NEK2 comprising contacting cells displaying over-expression of NEK2 or abnormally high NEK2 activity with at least one compound selected from compounds of Formula I, II, or III, or any of the compounds of the invention exemplified herein, or one or more of any of these in the form of a pharmaceutically acceptable salt.

The present invention further provides methods of treating a disease (e.g., cancer, for example, a hematologic cancer or solid tumor cancer) in a subject, wherein the disease is associated with NEK2 activity, comprising administering to the subject a therapeutically effective amount of at least one compound selected from Formula I, II, or III, or of any of the compounds of the invention exemplified herein, or one or more of any of these in the form of a pharmaceutically acceptable salt. In some embodiments, the cancer being treated is a hematologic cancer, for example, but not limited to, multiple myeloma, myelodysplastic syndrome (MDS), acute myelogenous leukemia (AML), acute lymphoblastic leukemia (ALL), acute lymphocytic leukemia, chronic lymphogenous leukemia, chronic lymphocytic leukemia (CLL), small lymphocytic lymphoma (SLL), mantle cell lymphoma, diffuse large B-cell lymphoma, follicular lymphoma, or non-Hodgkin's lymphoma. In some embodiments, the cancer being treated is a solid tumor cancer, for example, but not limited to, a tumor of the bones, digestive organs, reproductive organs, head, neck, lung, heart, skin, nervous system, endocrine system, neuroendocrine system, urinary system, soft tissue, or brain, for example, but not limited to, non-small cell lung cancer (NSCLC), colorectal carcinoma (CRC), ovarian cancer, melanoma, breast carcinoma, neurodocrine carcinoma, prostate adenocarcinoma, cholangiocarcinoma, uterine carcinoma, and pancreatic cancer.

A method of inhibiting the activity of NEK2 in a subject (e.g., patient) having a condition in which NEK2 has activity above normally functioning cells comprising administering an amount of at least one compound of any of Formula I, II, or III or of the compounds of the invention exemplified herein or at least one compound of any of these in the form of a pharmaceutically acceptable salt, in an amount sufficient to reduce the level of NEK2 activity.

A method of treating a disease in a subject, wherein the disease is associated with over-expression of NEK2 or high levels of NEK2 activity, comprising administering to the subject a therapeutically effective amount of at least one compound of any of Formula I, II, or III, or of the compounds of the invention exemplified herein or at least one compound of any of these in the form of a pharmaceutically acceptable salt, in an amount sufficient to reduce the level of NEK2 activity. In some embodiments the disease is cancer. In some embodiments, the cancer is a solid tumor cancer. In some embodiments, the cancer is a hematologic cancer.

In some embodiments wherein the cancer is a solid tumor cancer, it is a cancer of the bones, digestive organs, reproductive organs, head, neck, lung, heart, skin, nervous system, endocrine system, neuroendocrine system, urinary system, soft tissue, or brain, melanoma, renal cell cancer, non-small cell lung cancer (NSCLC), colorectal carcinoma (CRC), cervical cancer, ovarian cancer, melanoma, breast carcinoma, neurodocrine carcinoma, prostate cancer, cholangiocarcinoma, uterine carcinoma, neuroblastoma, peripheral nerve sheath tumor, testicular cancer, bladder cancer, pancreatic cancer or pancreatic cancer.

In some embodiments wherein the cancer is a hematologic cancer, the cancer is multiple myeloma, myelodysplastic syndrome (MDS), acute myelogenous leukemia (AML), acute lymphoblastic leukemia (ALL), acute lymphocytic leukemia, chronic lymphogenous leukemia, chronic lymphocytic leukemia (CLL), small lymphocytic lymphoma (SLL), mantle cell lymphoma, diffuse large B-cell lymphoma, follicular lymphoma, or non-Hodgkin's lymphoma.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments.

FIG. 1A shows the cell cycle analysis in SW480 cells after 24-hour treatment with MK-5108.

FIG. 1B shows the cell cycle analysis in SW480 cells after 24-hour treatment with AZD-1152.

FIG. 1C shows the cell cycle analysis in SW480 cells after 24-hour treatment with CMPD-16.

FIG. 1D shows the cell cycle analysis in SW480 cells after 24-hour treatment with CMPD-27.

FIG. 1E shows the cell cycle analysis in SW480 cells after 24-hour treatment with CMPD-12.

FIG. 1F shows the cell cycle analysis in SW480 cells after 24-hour treatment with CMPD-10.

FIG. 1G shows the cell cycle analysis in SW480 cells after 24-hour treatment with CMPD-07.

FIG. 1H shows EZH2 expression in SW480 cells after 24-hour treatment with selected compounds.

FIG. 2A is an image of a Western blot, and shows NEK2 inhibition suppresses activation of PP1α and HEC1.

FIG. 2B shows the ratio of pHEC1/HEC1 in the −TDTN lanes of the Western blot shown in FIG. 2A.

FIG. 2C shows the ratio of pHEC1/HEC1 in the +TDTN lanes of the Western blot shown in FIG. 2A.

FIG. 2D shows the ratio of pPP1α/PP1α in the −TDTN lanes of the Western blot shown in FIG. 2A.

FIG. 2E shows the ratio of pPP1α/PP1α in the +TDTN lanes of the Western blot shown in FIG. 2A.

FIG. 2F shows the ratio of pHistoneH3/HistoneH3 in the −TDTN lanes of the Western blot shown in FIG. 2A.

FIG. 2G shows the ratio of pHistoneH3/HistoneH3 in the +TDTN lanes of the Western blot shown in FIG. 2A.

### DETAILED DESCRIPTION

The present invention provides, inter alia, compounds of Formula I:

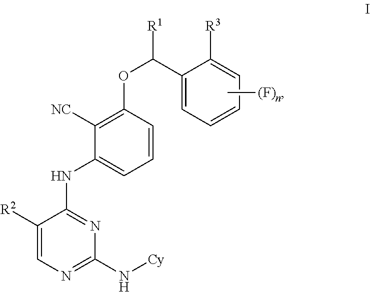

or a pharmaceutically acceptable salt thereof, wherein:

R¹ is —H or alkyl, in some embodiments, preferably, —H or methyl;

R² is alkoxy, alkyl, cycloalkyl, halo or —OH, in some embodiments, preferably, methoxy, —OH, chloro, methyl, ethyl, n-propyl, isopropyl, or cyclopropyl;

R³ is methoxy, H or fluoro, and when R³ is H or fluoro, n is 0 or 1, and when R³ is methoxy, n is 0; and

Cy is a moiety having the structure:

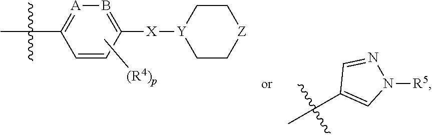

wherein:

A and B are independently selected from CH or N, with the proviso that when one of A or B is N the other is CH;

X is a bond, CH₂, or C(O);

R⁴ is alkoxy or halo, in some embodiments, preferably, methoxy or fluoro;

p is 0, 1 or 2, in some embodiments, preferably, 0 or 1;

Y is N or CH;

Z is NH, N(CH₂)₀₋₂CH₃, N—(CH₂)₁₋₃N(CH₃)₂, N(CH₂)₁₋₂OH or O; and

R⁵ is

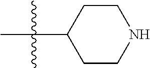 [SMILES: *C1CCNCC1]

or —(CH₂)₁₋₃N(CH₃)₂.

In some embodiments, R¹ is H.

In some embodiments, R² is chloro.

In some embodiments, R³ is fluoro.

In some embodiments, R³ is H and n is 1.

In some embodiments, Cy is:

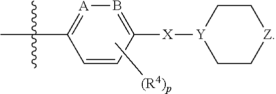

In some embodiments, A and B are each CH.

In some embodiments, p is 0.

In some embodiments, X is a bond.

In some embodiments, Y is N.

In some embodiments, Z is NH, NCH₃ or O.

In some embodiments, Z is NH.

In some embodiments, Z is NCH₂CH₂OH.

In some embodiments, Z is NCH₂CH₂N(CH₃)₂.

In some embodiments, Z is NCH₃.

In some embodiments, Z is O.

In some embodiments, R⁵ is CH₂CH₂N(CH₃)₂.

In some embodiments, R¹ is H; R² is chloro; R³ is fluoro and Cy is:

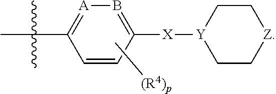

In some embodiments, the compounds of Formula I of the present invention have the structure of Formula II:

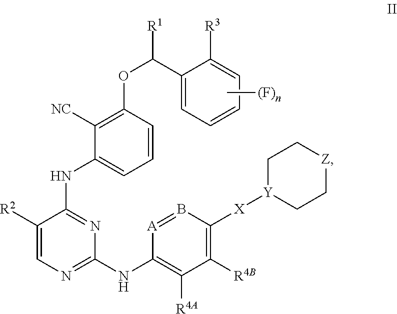

or a pharmaceutically acceptable salt thereof, wherein R^{4A} and R^{4B} are each independently selected from H, methoxy or fluoro.

In some embodiments, R^{4A} and R^{4B} are each H.

In some embodiments, the compounds of Formula I of the present invention have the structure of Formula III:

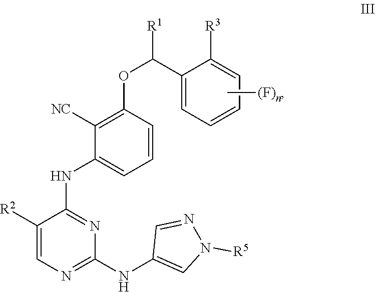

or a pharmaceutically acceptable salt thereof.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl. Likewise, the term “N(CH₂)₀₋₂CH₃” is specifically intended to individually disclose NCH₃, NCH₂CH₃, and NCH₂CH₂CH₃.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. Unless specified otherwise at the point of use herein, an alkyl group contains from 1 to 10 carbon atoms. In some embodiments, an alkyl group preferably contains from 1 to 8, from 1 to 6, from 1 to 4, or from 1 to 3 carbon atoms. The term “alkylenyl” refers to a bivalent alkyl group.

As used herein, “alkenyl” refers to an alkyl group having one or more double carbon-carbon bonds. Example alkenyl groups include ethenyl, propenyl, cyclohexenyl, and the like. The term “alkenylenyl” refers to a bivalent alkenyl group.

As used herein, “alkynyl” refers to an alkyl group having one or more triple carbon-carbon bonds. Example alkynyl groups include ethynyl, propynyl, and the like. The term “alkynylenyl” refers to a bivalent alkynyl group.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups, which can be poly-fused ring systems, can have from 6 to about 20 carbon atoms. Unless specified otherwise at the point of use herein, a monocyclic aryl has 5 to 8 ring members, and a polycyclic (e.g., fused) aryl has 8 to 10 ring members. In some embodiments, a monocyclic aryl preferably contains 6 ring members. In some embodiments, a polycyclic aryl preferably contains 10 ring members.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems. Also included in the definition of cycloalkyl are moieties that have one or more aromatic rings fused (i.e., having a bond in common with) to the cycloalkyl ring, for example, benzo derivatives of pentane, pentene, hexane, and the like. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. Unless specified otherwise at the point of use herein, a monocyclic cycloalkyl contains from 3 to 8 ring members, and a polycyclic (e.g., fused) cycloalkyl contains from 5 to 10 ring members. In some embodiments, a cycloalkyl (e.g., monocyclic cycloalkyl) preferably contains from 3 to 6, from 5 to 6, 5 or 6 ring members.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms. Unless specified otherwise at the point of use herein, a monocyclic heteroaryl has 5 to 8 ring members, and a polycyclic (e.g., fused) heteroaryl has 8 to 10 ring members. In some embodiments, a monocyclic aryl preferably contains from 5 to 6 ring members. In some embodiments, a polycyclic heteroaryl preferably contains 9 ring members.

As used herein, “heterocycloalkyl” refers to non-aromatic heterocycles containing at least one ring-forming heteroatom such as an O, N, or S atom. Heterocycloalkyl groups can be mono- or polycyclic (e.g., having 2, 3 or 4 fused rings) ring systems. Any ring-forming heteroatom or ring-forming carbon atom of a heterocycloalkyl group can also be oxidized by one or two oxo or sulfido substituents. Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 10 carbon atoms, and in further embodiments from about 3 to about 8 carbon atoms. In some embodiments, the heterocycloalkyl group contains 3 to about 7, 3 to about 6, or 3 to about 5 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds. Unless specified otherwise at the point of use herein, a monocyclic heterocycloalkyl contains from 3 to 8 ring members, and a polycyclic (e.g., fused) heterocycloalkyl contains from 5 to 10 ring members. In some embodiments, a heterocycloalkyl (e.g., monocyclic heterocycloalkyl) preferably contains from 3 to 6, from 5 to 6, 5 or 6 ring members.

As used herein, “halo” or “halogen” includes fluoro, chloro, bromo, and iodo. In some embodiments, halo is fluoro or chloro. In some embodiments, halo is fluoro. In some embodiments, halo is chloro.

As used herein, “alkoxy” refers to an —O-alkyl group. Example alkoxy groups include methoxy, ethoxy, propoxy (e.g., n-propoxy and isopropoxy), t-butoxy, and the like.

As used herein, “amino” refers to NH₂.

As used herein, “alkylamino” refers to an amino group substituted by an alkyl group.

As used herein, “dialkylamino” refers to an amino group substituted by two alkyl groups.

As used herein, “acyl” refers to —C(O)-alkyl.

As used herein, “acyloxy” refers to —OC(O)-alkyl.

As used herein, “acylamino” refers to an amino group substituted with an acyl group.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional crystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms, such as keto-enol tautomers.

As discussed below, compounds of the invention include all isotopes of atoms occurring in the intermediates or final compounds. Isotopes include those atoms having the same atomic number but different mass numbers. For example, isotopes of hydrogen include tritium and deuterium.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio, including those compounds which appear on the GRAS list as being recognized as acceptable for use in preparing pharmaceutical compositions for human use.

The present invention also includes pharmaceutically acceptable salts of the compounds described herein. As used herein, “salts” refers to derivatives of the disclosed compounds wherein the parent compound is modified by converting an existing acid or base moiety to its salt form. Examples of salts (e.g., pharmaceutically acceptable salts) include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods.

Depending on the process conditions, the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free form of the compound or another salt; a mixture of isomeric compounds of the present disclosure may be separated into the individual isomers.

Pharmaceutically acceptable salts are preferred. However, other salts may be useful, e.g., in isolation or purification steps which may be employed during preparation, and thus, are contemplated to be within the scope of the present disclosure.

As used herein, “pharmaceutically acceptable salts” refers to salts derived from suitable inorganic and organic acids and bases that are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable acid addition salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethanedisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate and xinafoate salts.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, or copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Examples of organic amines include, but are not limited to, isopropylamine, benzathine, cholinate, diethanolamine, diethylamine, lysine, meglumine, piperazine and tromethamine.

The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the relevant disclosure of which is hereby incorporated by reference in its entirety.

Compounds of the present disclosure that contain groups capable of acting as donors and/or acceptors for hydrogen bonds may be capable of forming co-crystals with suitable co-crystal formers. These co-crystals may be prepared from compounds of the present disclosure by known co-crystal forming procedures. Such procedures include grinding, heating, co-subliming, co-melting, or contacting in solution compounds of the present disclosure with the co-crystal former under crystallization conditions and isolating co-crystals thereby formed. Suitable co-crystal formers include those described in WO 2004/078163. Hence, the present disclosure further provides co-crystals comprising a compound of the present disclosure and a co-crystal former.

The present invention also includes prodrugs of the compounds described herein. As used herein, “prodrugs” refer to any compound which can act as a carrier to which an active pharmaceutical moiety, for example, a compound of the invention, is bonded, and from which the active pharmaceutical moiety is released when administered to a mammalian subject. Prodrugs can be prepared by modifying functional groups present in the compounds in such a way that the modifications are cleaved after administration to a subject (e.g., patient), producing in vivo the active pharmaceutical moiety. Prodrugs include, for example, but not limited to, compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

### Methods of Treatment Including Combinations

Compounds of the invention inhibit the activity of NEK2 (never in mitosis gene A-related kinase 2). In general, subjects (e.g., patients) needing treatment for a condition, for example, cancer, which treatment may benefit from inhibiting NEK2 activity therein, can benefit from administering a therapeutically effective amount (an NEK2-inhibiting amount) of a compound of the invention.

The compounds of the invention selectively inhibit NEK2 kinase. A compound which is “selective” demonstrates greater affinity for binding or inhibition of NEK2 than for binding or inhibition of other kinases. Without wishing to be bound by theory, it is believed that because inhibitors of NEK2 can promote anticancer effects, selectivity for NEK2 over other cell cycle-related proteins (e.g., other NEKs, aurora kinases or polo-like kinases), for example, the selectivity shown by the compounds of the invention described herein can offer the additional advantage of having fewer side effects and be particularly useful in the treatment of cancer. In some embodiments, the compounds of the invention are shown to be highly selective for NEK2 over the aurora kinases (e.g., Aurora A). Selectivity can be at least about 5-fold, at least about 10-fold, at least about 20-fold, at least about 50-fold, at least about 100-fold, at least about 200-fold, at least about 500-fold or at least about 1000-fold. Selectivity can be measured by methods routine in the art, for example, using assays described herein.

Another aspect of the present invention pertains to methods of treating or preventing (e.g., treating) a disease or disorder wherein selective inhibition of a NEK2 activity promotes a beneficial response to adjunct therapy or suppresses tumor proliferation or suppresses development of tumor resistance to therapeutic agents and is therefore associated with a desirable outcome in a subject (e.g., patient). Accordingly, in one aspect, treatment comprises administering to a subject in need of such treatment a therapeutically effective amount of at least one compound of the present invention in a free or pharmaceutically acceptable salt form. As used herein, a NEK2-associated disease can include any disease, disorder or condition that is directly or indirectly linked to over-expression or abnormally-high activity level of NEK2. A NEK2-associated disease can also include any disease, disorder or condition that can be prevented, ameliorated, or cured by in part modulating or inhibiting NEK2 activity.

Examples of NEK2-associated diseases include diseases involving uncontrolled cell proliferation due to the aberrant activity of various proteins, including NEK2 and/or its binding partners. In further embodiments, the NEK2-associated disease is cancer such as, for example, lung (e.g., non-small cell lung cancer or lung adenocarcinoma), breast, liver (e.g., hepatocellular), cervical, ovarian, prostate, leukemia, colorectal, neuroblastoma, peripheral nerve sheath tumor, testicular, bladder, pancreatic (e.g., pancreatic ductal adenocarcinoma), cholangiocarcinoma, renal, lymphoma, Ewing's sarcoma, glioblastoma, melanoma, cancers of the head and neck, mesothelioma, or multiple myeloma (Kokuryo, T. et al. Anticancer Research 2019, 39, 2251-2258; Franqui-Machin, R. et al. Journal of Clinical Investigation 2018, 128(7), 2877-2893). Examples of NEK2-associated diseases include hematologic cancers, for example, but not limited to, myeloma, leukemia or lymphoma. Further examples of NEK2-associated diseases are solid tumor cancers, such as breast, liver, pancreatic, or colorectal cancer. Other cancers are described herein, the treatment of which may be benefited by inhibition of NEK2 activity.

A wide variety of cancers, including solid tumor cancers, leukemias, lymphomas, and myelomas are amenable to the methods disclosed herein. In some embodiments, the cancer comprises a solid tumor (e.g., a colorectal, breast, prostate, lung, pancreatic, renal or ovarian tumor). Accordingly, in some embodiments, the cancer is a solid tumor cancer. In some embodiments, the cancer is selected from one or more of a cancer of the pulmonary system, a brain cancer, a cancer of the gastrointestinal tract, a skin cancer, a genitourinary cancer, head and neck cancer, a sarcoma, a carcinoma, and a neuroendocrine cancer. In various embodiments, the solid tumor cancer is breast cancer, bladder cancer, endometrial cancer, esophageal cancer, liver cancer, pancreatic cancer, lung cancer, cervical cancer, colon cancer, colorectal cancer, gastric cancer, kidney cancer, ovarian cancer, prostate cancer, testicular cancer, uterine cancer, a viral-induced cancer, melanoma or sarcoma. In some embodiments, the cancer is bladder cancer. In some embodiments, the cancer is lung cancer (e.g., non-small cell lung cancer). In other embodiments, the cancer is liver cancer. In some embodiments, the cancer is a sarcoma, bladder cancer or renal cancer. In some embodiments, the cancer is prostate cancer (e.g., castration-resistant prostate cancer, castration-sensitive prostate cancer). In other embodiments, the cancer is bladder cancer, pancreatic cancer, colorectal cancer, glioblastoma, kidney cancer, non-small cell lung carcinoma, prostate cancer, sarcoma, skin cancer, thyroid cancer, testicular cancer or vulvar cancer. In some embodiments, the cancer is endometrial cancer, pancreatic cancer, testicular cancer, renal cancer, melanoma, colorectal cancer, thyroid cancer, bladder cancer, pancreatic cancer, vulvar cancer, sarcoma, prostate cancer, lung cancer or anal cancer. In some embodiments, the cancer is a sarcoma. In some embodiments, the cancer is a renal cell carcinoma.

In some embodiments, the cancer is a hematologic cancer. Hematologic cancers that can be treated according to the methods described herein include leukemias (e.g., acute leukemias, chronic leukemias), lymphomas (e.g., B-cell lymphoma, T-cell lymphoma) and multiple myeloma. In some embodiments, the hematologic cancer is selected from multiple myeloma, myelodysplastic syndrome (MDS), acute myeloid leukemia (AML), acute lymphoblastic leukemia (ALL), acute lymphocytic leukemia, lymphocytic lymphoma, mycosis fungoides, chronic lymphogenous leukemia, chronic lymphocytic leukemia (CLL), mantle cell lymphoma, diffuse large B-cell lymphoma, follicular lymphoma, Hodgkin's lymphoma, non-Hodgkin's lymphoma or myelofibrosis.

In some embodiments, the cancer is a pre-metastatic cancer. In some embodiments, the cancer is a metastatic cancer.

Examples of cancer amenable to the methods described herein include, but are not limited to, adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, lung cancer (e.g., large cell lung cancer, such as squamous cell carcinoma, non-small cell lung), oat cell, papillary, scirrhous, bronchiolar, bronchogenic, squamous cell, and transitional cell). Additional examples of cancer amenable to the methods described herein include, but are not limited to, histiocytic disorders; leukemia; histiocytosis malignant; Hodgkin's disease; hypereosinophilia, immunoproliferative small; non-Hodgkin's lymphoma; plasmacytoma; reticuloendotheliosis; melanoma; chondroblastoma; chondroma; chondrosarcoma; dermatofibrosarcoma protuberans, fibrotic cancer (myelofibrosis, pancreatic cancer (e.g., pancreatic ductal adenocarcinoma), kidney cancer, liver cancer, lung cancer (e.g., large cell lung cancer, such as squamous cell carcinoma), breast cancer (e.g., inflammatory breast cancer), ovarian cancer (e.g., high grade serious ovarian carcinoma), endometrial cancer, uterine cancer, uterine sarcoma (e.g., uterine leiomyosarcoma), renal cell cancer, sarcoma (e.g., soft tissue sarcoma), malignant fibrous histiocytoma, fibrosarcoma (e.g., dermatofibrosarcoma protuberans) and hepatocellular carcinoma); fibroma; fibrosarcoma; giant cell tumors; histiocytoma; lipoma; liposarcoma; mesothelioma; myxoma; myxosarcoma; osteoma; osteosarcoma; pediatric malignancy, chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatocellular cancer, hepatoma; hidradenoma; islet cell tumor; Leydig cell tumor; papilloma; sertoli cell tumor; theca cell tumor; leiomyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. Yet more examples of cancer treatable according to the methods described herein include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Further examples of cancers amenable to the methods described herein include, but are not limited to, Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Cancer of the anal region; Anal Cancer; Appendix Cancer; Astrocytomas, Childhood; Atypical Teratoid/Rhabdoid Tumor, Childhood, Central Nervous System (CNS); Neoplasms of the CNS (e.g., primary CNS lymphoma, spinal axis tumors, medulloblastoma, brain stem gliomas or pituitary adenomas), Barrett's esophagus (e.g., pre-malignant syndrome), and mycoses fungoides, Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CIVIL); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sézary Syndrome); Ductal Carcinoma In Situ (DCIS); Embryonal Tumors, Central Nervous System, Childhood; Cancer of the Endocrine system (e.g., cancer of the thyroid, pancreas, parathyroid or adrenal glands), Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Gynecologic Tumors ((e.g., uterine sarcomas, carcinoma of the fallopian tubes, carcinoma of the endometrium, carcinoma of the cervix, carcinoma of the vagina or carcinoma of the vulva), Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Cutaneous or Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sézary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis (e.g., renal cell carcinoma, carcinoma of the renal pelvis), benign prostatic hypertrophy, parathyroid cancer, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated and/or prevented (e.g., treated) in accordance with the methods described herein.

Enhancer of zeste homolog 2 (EZH2) is a member of the polycomb group of genes, which are factors in the regulation of cell proliferation and differentiation. EZH2 expression has been associated with high proliferation rate and aggressive tumor subgroups in cutaneous melanoma, cancers of the endometrium, prostate and breast, and thyroid cancer. Bachmann, I. M., et al., J. Clin. Oncol. 24(2006):268-273; and Masudo, K., et al., in vivo 32:25-31 (2018). Overexpression of EZH2 and mutations to EZH2 resulting in gains of function have been shown to be particularly oncogenic. Kim, K. H. and Roberts, C. W. M., Nat. Med. 2016 February; 22(2):128-134. It has been shown that inhibition of EZH2 prevents NEK2 from antagonizing cellular senescence in NEK2-overexpressing multiple myeloma cells. See Zhu, Y., et al., Blood (2019), 134 (Supplement 1): 3102. NEK2-mediated signaling has also been observed in glioblastoma core cells, where NEK2 has been observed to bind with EZH2 and prevent proteasome-mediated degradation of EZH2. Wang, J., et al., bioRxiv https://doi.org/10.1101/2020.12.01.405696 (2020). Described herein are data that show that certain NEK2 inhibitors selectively suppress EZH2 expression, suggesting that reduced EZH2 expression or activity may be indicative of NEK2 inhibition and, potentially, may be useful as a biomarker for NEK2-associated diseases and disorders, and/or as a biomarker for treatment response or efficacy of a therapy comprising a NEK2 inhibitor compound disclosed herein.

Accordingly, also provided herein is a method of modulating (e.g., inhibiting) the expression or activity (e.g., expression) of EZH2 in a subject (e.g., patient), comprising administering an amount of at least one compound of any of Formula I, II, or III or of the compounds of the invention exemplified herein or at least one compound of any of these in the form of a pharmaceutically acceptable salt, in an amount sufficient to modulate (e.g., reduce) the expression or activity (e.g., expression) of EZH2. In some embodiments, the expression or activity of EZH2 is modulated (e.g., reduced) without affecting cell cycle (e.g., without inducing cell cycle arrest).

Also provided herein is a method of modulating (e.g., inhibiting) the expression or activity (e.g., expression) of EZH2 in a subject (e.g., patient) having a condition in which EZH2 is expressed aberrantly (e.g., overexpressed) or has activity different from (e.g., above) normally functioning cells, comprising administering an amount of at least one compound of any of Formula I, II, or III or of the compounds of the invention exemplified herein or at least one compound of any of these in the form of a pharmaceutically acceptable salt, in an amount sufficient to modulate (e.g., reduce) the expression or activity (e.g., expression) of EZH2.

Also provided herein is a method of treating a disease in a subject, wherein the disease is associated with expression (e.g., overexpression) or activity (e.g., increased activity) of EZH2, comprising administering to the subject a therapeutically effective amount of at least one compound of any of Formula I, II, or III, or of the compounds of the invention exemplified herein or at least one compound of any of these in the form of a pharmaceutically acceptable salt (e.g., an amount sufficient to reduce the expression or activity of EZH2). In some embodiments the disease is cancer. In some embodiments, the cancer is a solid tumor cancer. In some embodiments, the cancer is a hematologic cancer.

Also provided is a method of treating a disease or disorder described herein (e.g., a NEK2-associated disease or disorder, an EZH2-associated disease or disorder) in a subject in need thereof, comprising administering to the subject a therapeutically effective amount of at least one compound of the present invention in a free or pharmaceutically acceptable salt form, wherein the subject has been identified as having a biomarker associated with the disease or disorder. For example, it is thought that aberrantly expressed (e.g., overexpressed and/or mutated) and/or aberrantly active (e.g., overactive) EZH2 may serve as a biomarker indicative of a NEK2-associated disease or disorder.

EZH2 may also serve as a biomarker for treatment response or efficacy of a therapy comprising a NEK2 inhibitor compound disclosed herein, and expression or activity of EZH2 may serve as an indicator of (e.g., may correlate with) NEK2 expression or activity. Thus, in some embodiments of a method described herein, the method further comprises measuring (e.g., monitoring) the expression or activity of EZH2 in the subject. In some embodiments, the method further comprises measuring (e.g., monitoring) expression or activity of EZH2 in the subject, thereby determining efficacy of the compound of the present disclosure in the subject. For example, a reduction in the expression or activity of EZH2 (e.g., over time, as over the course of treatment, or as measured before and during and/or after treatment or before or during and after treatment) can be indicative of inhibition of NEK2 and/or efficacy of a compound of the present disclosure in the subject. A measured level of expression or activity of EZH2 can also or alternatively be compared to an appropriate control (e.g., based on a population of healthy subjects) to provide an indication of inhibition of NEK2 and/or efficacy of a compound of the present disclosure in the subject.

As used herein, the term “contacting” refers to the bringing the indicated moieties together within the same chemical environment in an in vitro system or an in vivo system. For example, “contacting” NEK2 with a compound of the invention includes the administration of at least one compound of the present invention to an individual or subject (e.g., patient), such as a human, in need of inhibition of having NEK2 activity.

As used herein, the terms “subject” and “individual”, used interchangeably, refer to any animal, including mammals, preferably mice, rats, other rodents, rabbits, dogs, cats, swine, cattle, sheep, horses, or primates, and most preferably humans. “Subject” and “individual” also include “patients,” humans in need of a treatment. In some embodiments herein, a subject is a human, e.g., a patient.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease state; for example, preventing a disease, condition or disorder in an individual who may be predisposed to the disease, condition or disorder but does not yet experience or display the pathology or symptomatology of the disease;

(2) inhibiting or modulating the disease; for example, inhibiting a disease, condition or disorder in an individual who is experiencing or displaying the pathology or symptomatology of the disease, condition or disorder (i.e., reducing one or more symptoms of the disease or arresting further development of the pathology and/or symptomatology);

(3) ameliorating the disease; for example, ameliorating a disease, condition or disorder in an individual who is experiencing or displaying the pathology or symptomatology of the disease, condition or disorder (i.e., reversing the pathology and/or symptomatology); and

(4) reversing some aspect of the disease or a mechanistic pathway by which the disease progresses which permits further therapeutic treatment by other medicaments, for example, reversing drug resistance in a cancer, reducing the rate of proliferation of the cancer or interrupting a pathway by which a cancer mutates during the progression of the disease.

Compounds of the invention may be employed with one or more additional pharmaceutical agents to effectuate a full treatment response such as is described below. For example, a NEK2 inhibitor can be used in combination with a chemotherapeutic in the treatment of a solid tumor cancer (for example, but not limited to breast cancer, multiple myeloma, hepatocellular carcinoma and pancreatic ductal adenocarcinoma), or a hematologic cancer, for example, but not limited to multiple myeloma, myelodysplastic syndrome (MDS), acute myelogenous leukemia (AML), acute lymphoblastic leukemia (ALL), acute lymphocytic leukemia, chronic lymphogenous leukemia, chronic lymphocytic leukemia (CLL), or small lymphocytic lymphoma (SLL).

### Combination Therapies

Examples of pharmaceutical agents used in the treatment and/or prevention (e.g., treatment) of cancer, for example, multiple myeloma, can include, without limitation, bortezomib (Velcade), P5091, melphalan, melphalan plus prednisone, doxorubicin, and dexamethasone. Additive or synergistic effects are desirable outcomes of combining a NEK2 inhibitor of the present invention with an additional agent. Furthermore, resistance of multiple myeloma and other cancer cells to agents such as bortezomib, 5-fluorouracil, tamoxifen, trastuzumab, paclitaxel and doxorubicin may be reversible upon treatment with a NEK2 inhibitor of the present invention (Kokuryo, T. et al. Anticancer Research 2019, 39, 2251-2258; Franqui-Machin, R. et al. Journal of Clinical Investigation 2018, 128(7), 2877-2893). The agents can be combined with the present compounds in a single or continuous dosage form, or the agents can be administered simultaneously or sequentially as separate dosage forms.

In still another embodiment, at least one compound of Formula I, II, III or any of the exemplified compounds of the invention, or one or more of any of these in the form of a pharmaceutically acceptable salt, is administered to a subject in need thereof in combination with a B-cell receptor signaling antagonist (e.g., a Bruton's tyrosine kinase (BTK) inhibitor, such as Ibrutinib). Accordingly, methods of the present disclosure include methods for treating cancer comprising administering a therapeutically effective amount of at least one compound of Formula I, II, III or any of the exemplified compounds of the invention, or one or more of any of these in the form of a pharmaceutically acceptable salt and a Bruton's tyrosine kinase (BTK) inhibitor to a subject in need thereof. In some embodiments, administration of at least one compound of Formula I, II, III or any of the exemplified compounds of the invention, or one or more of any of these in the form of a pharmaceutically acceptable salt, is administered before administration of the B-cell receptor signaling antagonist (e.g., the BTK inhibitor). In some embodiments, administration of at least one compound of Formula I, II, III or any of the exemplified compounds of the invention, or any of these in the form of a pharmaceutically acceptable salt, is administered concurrently with (contemporaneously with) administration of the B-cell receptor signaling antagonist (e.g., the BTK inhibitor). In some embodiments, administration of a compound of at least one compound Formula I, II, III or any of the exemplified compounds of the invention, or any of these in the form of a pharmaceutically acceptable salt, is administered after administration of the B-cell receptor signaling antagonist (e.g., the BTK inhibitor).

In various embodiments, the BTK inhibitor is ibrutinib. In some particular embodiments, the cancer is chronic lymphocytic leukemia (CLL), small lymphocytic lymphoma (SLL), or both. In some embodiments, the subject has received a prior treatment regimen for CLL, SLL, or both. In some embodiments, the subject was refractory after the prior treatment regimen, the subject has relapsed CLL, SLL, or both after a response to the prior treatment regimen, or the subject has detectable minimal residual disease (MRD).

In some embodiments, at least one compound of Formula I, II, III or any one or more of the exemplified compounds of the invention, or any of these in the form of a pharmaceutically acceptable salt, is administered to a subject in need thereof in combination with a Bcl-2 inhibitor, for example, venetoclax, for example, in the treatment of leukemia (e.g., CLL, SLL, or both). The administration may be before, concurrently (contemporaneously with) or after administration of the Bcl-2 inhibitor. Such treatment may be carried out even when a subject was insensitive to treatment with a Bcl-2 inhibitor, is ineligible for treatment with a Bcl-2 inhibitor or has relapsed after treatment with a Bcl-2 inhibitor.

Immunomodulators may be employed along with one or more compounds of Formula I, II, III or any of the exemplified compounds of the invention, or one or more of any of these in the form of a pharmaceutically acceptable salt. Immunomodulators of particular interest for use in combination with compounds of the present disclosure include: afutuzumab (available from ROCHE®); pegfilgrastim (NEULASTA®); lenalidomide (CC-5013, REVLIMID®); thalidomide (THALOMID®); actimid (CC4047); and IRX-2 (mixture of human cytokines including interleukin 1, interleukin 2, and interferon γ, CAS 951209-71-5, available from IRX Therapeutics).

Chimeric Antigen Receptor T-Cell (CAR-T) therapies may be employed along with one or more compounds of Formula I, II, III or any of the exemplified compounds of the invention, or any of these in the form of a pharmaceutically acceptable salt. CAR-T therapies of particular interest for use in combination with compounds of the present disclosure include: Tisagenlecleucel (Novartis), Axicabtagene ciloleucel (Kite), and Tocilizumab and Atlizumab (Roche).

In some embodiments, administration of at least one compound of Formula I, II, III or any of the exemplified compounds of the invention, or one or more of any of these in the form of a pharmaceutically acceptable salt (NEK2 inhibitor of the invention), is administered to a subject in need thereof in combination with an immune checkpoint inhibitor (e.g., a PD-1 inhibitor (such as Pembrolizumab or Nivolumab), a PD-L1 inhibitor (such as Atezolizumab, Avelumab, or Durvalumab), a CTLA-4 inhibitor, a LAG-3 inhibitor, or a Tim-3 inhibitor). Accordingly, methods of the present disclosure include methods for treating cancer comprising administering an effective amount of a NEK2 inhibitor of the invention and an immune checkpoint inhibitor to a subject in need thereof. The administration of the NEK2 inhibitor of the invention may be before, concurrently or after administration of the immune checkpoint inhibitor (e.g., a PD-1 inhibitor (such as Pembrolizumab or Nivolumab), a PD-L1 inhibitor (such as Atezolizumab, Avelumab, or Durvalumab), a CTLA-4 inhibitor, a LAG-3 inhibitor, or a Tim-3 inhibitor).

In some embodiments, the NEK2 inhibitor of the invention and immune checkpoint inhibitor are co-administered. In other embodiments, the NEK2 inhibitor of the invention is administered after the immune checkpoint inhibitor. In still different embodiments, the NEK2 inhibitor of the invention is administered before the immune checkpoint inhibitor.

Immune checkpoint inhibitors of interest for use in combination with compounds of the present disclosure include: PD-1 inhibitors, such as pembrolizumab (KEYTRUDA®), nivolumab (OPDIVO®), cemiplimab (LIBTAYO®), spartalizumab (PDR001), Pidilizumab (CureTech), MEDI0680 (Medimmune), cemiplimab (REGN2810), dostarlimab (TSR-042), PF-06801591 (Pfizer), tislelizumab (BGB-A317), camrelizumab (INCSHR1210, SHR-1210), and AMP-224 (Amplimmune); PD-L1 inhibitors, such as atezolizumab (TECENTRIQ®), avelumab (BAVENCIO®), durvalumab (IMFINZI®), FAZ053 (Novartis), and BMS-936559 (Bristol-Myers Squibb); and drugs that target CTLA-4, such as ipilimumab (YERVOY®).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy in combination with compounds of the present disclosure. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

In various embodiments, the immune checkpoint inhibitor is a PD-1 inhibitor. In specific embodiments, the PD-1 inhibitor is Pembrolizumab, Nivolumab, or a combination thereof. In particular embodiments, the PD-1 inhibitor is Pembrolizumab (also known as Lambrolizumab, MK-3475, MK03475, SCH-900475, or KEYTRUDA®). Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In particular embodiments, the PD-1 inhibitor is Nivolumab (also known as MDX-1106, MDX-1106-04, ONO-4538, BMS-936558, or OPDIVO®). Nivolumab (clone 5C4) and other anti-PD-1 antibodies are disclosed in U.S. Pat. No. 8,008,449 and WO 2006/121168, incorporated by reference in their entirety. In some other embodiments, the PD-1 inhibitor is AMP-224 (Amplimmune), CBT-501 (CBT Pharmaceuticals), CBT-502 (CBT Pharmaceuticals), JS001 (Junshi Biosciences), IBI308 (Innovent Biologics), INCSHR1210 (Incyte), also known as SHR-1210 (Hengrui Medicine), BGBA317 (Beigene), BGB-108 (Beigene), BAT-I306 (Bio-Thera Solutions), GLS-010 (Gloria Pharmaceuticals; WuXi Biologics), AK103, AK104, AK105 (Akesio Biopharma; Hangzhou Hansi Biologics; Hanzhong Biologics), LZMO09 (Livzon), HLX-10 (Henlius Biotech), MEDI0680 (Medimmune), PDF001 (Novartis), PF-06801591 (Pfizer), Pidilizumab (CureTech), REGN2810 (Regeneron), TSR-042 (Tesaro) also known as ANB011, or CS1003 (CStone Pharmaceuticals). MEDI0680 (Medimmune), is also known as AMP-514 MEDI0680 and other anti-PD-1 antibodies are disclosed in U.S. Pat. No. 9,205,148 and WO 2012/145493, incorporated by reference in their entirety. Pidilizumab is also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety.

In one embodiment, the anti-PD-1 antibody molecule is Cemiplimab. In one embodiment, the anti-PD-1 antibody molecule is Sintilimab. In one embodiment, the anti-PD-1 antibody molecule is Toripalimab. In one embodiment, the anti-PD-1 antibody molecule is Camrelizumab.

Further known anti-PD-1 antibody molecules include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In one embodiment, the anti-PD-1 antibody molecule comprises the CDRs, variable regions, heavy chains and/or light chains of BAP049-Clone-E or BAP049-Clone-B disclosed in US 2015/0210769. The antibody molecules described herein can be made by vectors, host cells, and methods described in US 2015/0210769, incorporated by reference in its entirety.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

In some embodiments, the immune checkpoint inhibitor is a PD-L1 inhibitor. In some such embodiments, the PD-L1 inhibitor is Atezolizumab, Avelumab, Durvalumab, or a combination thereof. In particular embodiments, the PD-L1 inhibitor is Atezolizumab also known as MPDL3280A, RG7446, RO5541267, YW243.55.570, or TECENTRIQ™. Atezolizumab and other anti-PD-L1 antibodies are disclosed in U.S. Pat. No. 8,217,149, incorporated by reference in its entirety. In particular embodiments, the PD-L1 inhibitor is Avelumab also known as MSB0010718C. Avelumab and other anti-PD-L1 antibodies are disclosed in WO 2013/079174, incorporated by reference in its entirety. In particular embodiments, the PD-L1 inhibitor is Durvalumab also known as MEDI4736. Durvalumab and other anti-PD-L1 antibodies are disclosed in U.S. Pat. No. 8,779,108, incorporated by reference in its entirety. In certain embodiments, the PD-L1 inhibitor is KN035 (Alphamab; 3DMed), BMS 936559 (Bristol-Myers Squibb), CS1001 (CStone Pharmaceuticals), FAZ053 (Novartis), SHR-1316 (Hengrui Medicine), TQB2450 (Chiatai Tianqing), STI-A1014 (Zhaoke Pharm; Lee's Pharm), BGB-A333 (Beigene), MSB2311 (Mabspace Biosciences), or HLX-20 (Henlius Biotech). In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In some embodiments, the PD-L1 inhibitor is a monoclonal antibody (e.g., as made by Hisun Pharm and applying for clinical trials as of this filing).

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety. In one embodiment, the anti-PD-L1 antibody molecule comprises the CDRs, variable regions, heavy chains and/or light chains of BAP058-Clone 0 or BAP058-Clone N disclosed in US 2016/0108123.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In some embodiments, the immune checkpoint inhibitor is a CTLA-4 inhibitor. In certain embodiments, the CTLA-4 inhibitor is ipilimumab. In other embodiments, the CTLA4 inhibitor is tremelimumab.

In some embodiments, the immune checkpoint inhibitor is a LAG-3 inhibitor. In some embodiments, the LAG-3 inhibitor is chosen from LAG525 (Novartis), BMS-986016 (Bristol-Myers Squibb), or TSR-033 (Tesaro).

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety. In one embodiment, the anti-LAG-3 antibody molecule comprises the CDRs, variable regions, heavy chains and/or light chains of BAP050-Clone I or BAP050-Clone J disclosed in US 2015/0259420.

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule is TSR-033 (Tesaro). In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule is IMP761 (Prima BioMed).

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 inhibitor is a soluble LAG-3 protein, e.g., IMP321 (Prima BioMed), e.g., as disclosed in WO 2009/044273, incorporated by reference in its entirety.

In some embodiments, the immune checkpoint inhibitor is a TIM-3 inhibitor. In some embodiments, the TIM-3 inhibitor is MGB453 (Novartis) or TSR-022 (Tesaro).

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled“Antibody Molecules to TIM-3 and Uses Thereof,” incorporated by reference in its entirety. In one embodiment, the anti-TIM-3 antibody molecule comprises the CDRs, variable regions, heavy chains and/or light chains of ABTIM3-hum11 or ABTIM3-hum03 disclosed in US 2015/0218274.

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121. APE5137, APE5121, and other anti-TIM-3 antibodies are disclosed in WO 2016/161270, incorporated by reference in its entirety. In one embodiment, the anti-TIM-3 antibody molecule is the antibody clone F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In some embodiments, at least one compound of Formulae I, II, III or any of the exemplified compounds of the invention, or one or more of any of these in the form of a pharmaceutically acceptable salt, is administered to a subject in need thereof in combination with a bromodomain inhibitor, a histone deacetylase (HDAC), or both.

A bromodomain inhibitor inhibits at least one bromodomain protein, such as Brd2, Brd3, Brd4 and/or BrdT, for example Brd4. In some of these embodiments, the bromodomain inhibitor is JQ-1 (Nature 2010 Dec. 23; 468(7327):1067-73), BI2536 (ACS Chem. Biol. 2014 May 16; 9(5):1160-71; Boehringer Ingelheim), TG101209 (ACS Chem. Biol. 2014 May 16; 9(5):1160-71), OTX015 (Mol. Cancer Ther. November 201312; C244; Oncoethix), IBET762 (J Med Chem. 2013 Oct. 10; 56(19):7498-500; GlaxoSmithKline), IBET151 (Bioorg. Med. Chem. Lett. 2012 Apr. 15; 22(8):2968-72; GlaxoSmithKline), PFI-1 (J. Med. Chem. 2012 Nov. 26; 55(22):9831-7; Cancer Res. 2013 Jun. 1; 73(11):3336-46; Structural Genomics Consortium) of CPI-0610 (Constellation Pharmaceuticals). In some embodiments, the bromodomain inhibitor is TG101209, BI2536, OTX015, C244, IBET762, IBET151, or PFI-1.

A HDAC inhibitor inhibits at least one HDAC protein. HDAC proteins may be grouped into classes based on homology to yeast HDAC proteins with Class I made up of HDAC1, HDAC2, HDAC3 and HDAC 8; Class IIa made up of HDAC4, HDAC5, HDAC7 and HDAC 9; Class IIb made up of HDAC6 and HDAC10; and Class IV made up of HDAC11. In some of these embodiments, the HDAC inhibitor is trichostatin A, vorinostat (Proc. Natl. Acad. Sci. U.S.A. 1998 Mar. 17; 95(6):3003-7), givinostat, abexinostat (Mol. Cancer Ther. 2006 May; 5(5):1309-17), belinostat (Mol. Cancer Ther. 2003 August; 2(8):721-8), panobinostat (Clin. Cancer Res. 2006 Aug. 1; 12(15):4628-35), resminostat (Clin. Cancer Res. 2013 Oct. 1; 19(19):5494-504), quisinostat (Clin. Cancer Res. 2013 Aug. 1; 19(15):4262-72), depsipeptide (Blood. 2001 Nov. 1; 98(9):2865-8), entinostat (Proc. Natl. Acad. Sci. U.S.A. 1999 Apr. 13; 96(8):4592-7), mocetinostat (Bioorg. Med. Chem. Lett. 2008 Feb. 1; 18(3):106771) or valproic acid (EMBO J. 2001 Dec. 17; 20(24):6969-78). For example, in some embodiments the HDAC inhibitor is panobinostat, vorinostat, MS275, belinostat, or LBH589. In some embodiments, the HDAC inhibtior is panobinostat or SAHA.

In some embodiments, methods of the present disclosure further comprise administering radiation therapy to the subject.

Some patients may experience allergic reactions to compounds of the present disclosure and/or other therapeutic agent(s) (e.g., anti-cancer agent(s)) during or after administration. Therefore, anti-allergic agents can be administered in combination with compounds of the present disclosure and/or other therapeutic agent(s) (e.g., anti-cancer agent(s)) to minimize the risk of an allergic reaction. Suitable anti-allergic agents include corticosteroids (Knutson, S., et al., PLoS One, DOI:10.1371/journal.pone.0111840 (2014)), such as dexamethasone (e.g., DECADRON®), beclomethasone (e.g., BECLOVENT®), hydrocortisone (also known as cortisone, hydrocortisone sodium succinate, hydrocortisone sodium phosphate, sold under the tradenames ALA-CORT®, hydrocortisone phosphate, SOLU-CORTEF®, HYDROCORT ACETATE® and LANACORT®), prednisolone (sold under the tradenames DELTA-CORTEL®, ORAPRED®, PEDIAPRED® and PRELONE®), prednisone (sold under the tradenames DELTASONE®, LIQUID RED®, METICORTEN® and ORASONE®), methylprednisolone (also known as 6-methylprednisolone, methylprednisolone acetate, methylprednisolone sodium succinate, sold under the tradenames DURALONE®, MEDRALONE®, MEDROL®, M-PREDNISOL® and SOLU-MEDROL®); antihistamines, such as diphenhydramine (e.g., BENADRYL®), hydroxyzine, and cyproheptadine; and bronchodilators, such as the beta-adrenergic receptor agonists, albuterol (e.g., PROVENTIL®), and terbutaline (BRETHINE®).

Some patients may experience nausea during and after administration of the compounds described herein and/or other therapeutic agent(s) (e.g., anti-cancer agent(s)). Therefore, anti-emetics can be used in combination with compounds of the present disclosure and/or other therapeutic agent(s) (e.g., anti-cancer agent(s)) to prevent nausea (upper stomach) and vomiting. Suitable anti-emetics include aprepitant (EMEND®), ondansetron (ZOFRAN®), granisetron HCl (KYTRIL®), lorazepam (ATIVAN®, dexamethasone (DECADRON®), prochlorperazine (COMPAZINE®), casopitant (REZONIC® and ZUNRISA®), and combinations thereof.

Medication to alleviate the pain experienced during the treatment period is often prescribed to make the patient more comfortable. Common over-the-counter analgesics, such TYLENOL®, can also be used in combination with compounds of the present disclosure and/or other therapeutic agent(s) (e.g., anti-cancer agent(s)). Opioid analgesic drugs such as hydrocodone/paracetamol or hydrocodone/acetaminophen (e.g., VICODIN®), morphine (e.g., ASTRAMORPH® or AVINZA®), oxycodone (e.g., OXYCONTIN® or PERCOCET®), oxymorphone hydrochloride (OPANA®), and fentanyl (e.g., DURAGESIC®) can be useful for moderate or severe pain, and can be used in combination with compounds of the present disclosure and/or other therapeutic agent(s) (e.g., anti-cancer agent(s)).

In some of the foregoing embodiments, the method is for treating liver cancer, refractory cancers (e.g., non-small cell lung cancer), lung cancer, esophageal cancer, Hodgkin's lymphoma, NK/T-cell lymphoma, or melanoma. In some specific embodiments, the method is for treating esophageal squamous cell carcinoma, gastric cancer, lung cancer, nasopharyngeal carcinoma, bladder cancer, soft tissue sarcoma, diffuse large B-cell lymphoma, head and neck squamous cell carcinomas, kidney cancer, urothelial carcinoma, ovarian cancer, uterine cancer, or pancreatic cancer.

Other embodiments provide methods for combination therapies in which an agent known to modulate other pathways, or other components of the same pathway, or even overlapping sets of target enzymes are used in combination with a compound of comprising administering an effective amount of at least one compound of Formula I, II, III or any of the exemplified compounds of the invention, or one or more of any of these in the form of a pharmaceutically acceptable salt with chemotherapeutic agents, therapeutic antibodies, and radiation treatment, to provide a synergistic or additive therapeutic effect.

Many chemotherapeutics are presently known in the art and can be used in combination with a compound of Formula I, II, III or any of the exemplified compounds of the invention, or one or more of any of these in the form of a pharmaceutically acceptable salt. In some embodiments, the chemotherapeutic is selected from the group consisting of mitotic inhibitors, alkylating agents, hypomethylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Non-limiting examples of therapeutic agents that can be used in combinations with a compound of Formula I, II, III or any of the exemplified compounds of the invention, or one or more of any of these in the form of a pharmaceutically acceptable salt are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex®, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.RTM.; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above.

Examples of chemotherapeutic agents for use in combination with a compound of Formula I, II, III or any of the exemplified compounds of the invention, or one or more of any of these in the form of a pharmaceutically acceptable salt (e.g., in combination therapy, in a pharmaceutical combination) include capecitabine (Xeloda®), N4-pentoxycarbonyl-5-deoxy-5-fluorocytidine, carboplatin (Paraplatin®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), doxorubicin hydrochloride (Adriamycin®, Rubex®), fludarabine phosphate (Fludara®), 5-fluorouracil (Adrucil®, Efudex®), gemcitabine (difluorodeoxycitidine), irinotecan (Camptosar®), L-asparaginase (ELSPAR®), 6-mercaptopurine (Purinethol®), methotrexate (Folex®), pentostatin, 6-thioguanine, thiotepa, and topotecan hydrochloride for injection (Hycamptin®).

Anti-cancer agents of particular interest for use in combination with the compounds of the present disclosure include:

Purine antimetabolites and/or inhibitors of de novo purine synthesis: pemetrexed (Alimta®), gemcitabine (Gemzar®), 5-fluorouracil (Adrucil®, Carac® and Efudex®), methotrexate (Trexall®), capecitabine (Xeloda®), floxuridine (FUDR®), decitabine (Dacogen®), azacitidine (Vidaza® and Azadine®), 6-mercaptopurine (Purinethol®), cladribine (Leustatin®, Litak® and Movectro®), fludarabine (Fludara®), pentostatin (Nipent®), nelarabine (Arranon®), clofarabine (Clolar® and Evoltra®), and cytarabine (Cytosar®).

MTAP inhibitors: (3R,4S)-1-((4-amino-5H-pyrrolo[3,2-d]pyrimidin-7-yl)methyl)-4-((methylthio)methyl)pyrrolidin-3-ol (MT-DADMe-Immucillin-A, CAS 653592-04-2).

Methylthioadenosine: ((2R,3R,4S,5S)-2-(6-amino-9H-purin-9-yl)-5-((methylthio)methyl)tetrahydrofuran-3,4-diol, CAS 2457-80-9).

MET inhibitors: capmatinib (INC280, CAS 1029712-80-8).

Platelet-derived growth factor (PDGF) receptor inhibitors: imatinib (Gleevec®); linifanib (N-[4-(3-amino-1H-indazol-4-yl)phenyl]-N′-(2-fluoro-5-methylphenyl)urea, also known as ABT 869, available from Genentech); sunitinib malate (Sutent®); quizartinib (AC220, CAS 950769-58-1); pazopanib (Votrient®); axitinib (Inlyta®); sorafenib (Nexavar®); vargatef (BIBF1120, CAS 928326-83-4); telatinib (BAY57-9352, CAS 332012-40-5); vatalanib dihydrochloride (PTK787, CAS 212141-51-0); and motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470).

Phosphoinositide 3-kinase (PI3K) inhibitors: 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 4-(trifluoromethyl)-5-(2,6-dimorpholinopyrimidin-4-yl)pyridin-2-amine (also known as BKM120 or NVP-BKM120, and described in PCT Publication No. WO 2007/084786); alpelisib (BYL719): (5Z)-5-[[4-(4-Pyridinyl)-6-quinolinyl]methylene]-2,4-thiazolidinedione (GSK1059615, CAS 958852-01-2); 5-[8-methyl-9-(1-methylethyl)-2-(4-morpholinyl)-9H-purin-6-yl]-2-pyrimidinamine (VS-5584, CAS 1246560-33-7) and everolimus (AFINITOR®).

Cyclin-dependent kinase (CDK) inhibitors: ribociclib (LEE011, CAS 1211441-98-3); aloisine A; alvocidib (also known as flavopiridol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002); crizotinib (PF-02341066, CAS 877399-52-5); 2-(2-chlorophenyl)-5,7-dihydroxy-8-[(2R,3S)-2-(hydroxymethyl)-1-methyl-3-pyrrolidinyl]-4H-1-benzopyran-4-one, hydrochloride (P276-00, CAS 920113-03-7); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265, CAS 927880-90-8); indisulam (E7070); roscovitine (CYC202); 6-acetyl-8-cyclopentyl-5-methyl-2-(5-piperazin-1-yl-pyridin-2-ylamino)-8H-pyrido[2,3-d]pyrimidin-7-one, hydrochloride (PD0332991); dinaciclib (SCH727965); N-[5-[[(5-tert-butyloxazol-2-yl)methyl]thio]thiazol-2-yl]piperidine-4-carboxamide (BMS 387032, CAS 345627-80-7); 4-[[9-chloro-7-(2,6-difluorophenyl)-5H-pyrimido[5,4-d][2]benzazepin-2-yl]amino]-benzoic acid (MLN8054, CAS 869363-13-3); 5-[3-(4,6-difluoro-1H-benzimidazol-2-yl)-1H-indazol-5-yl]-N-ethyl-4-methyl-3-pyridinemethanamine (AG-024322, CAS 837364-57-5); 4-(2,6-dichlorobenzoylamino)-1H-pyrazole-3-carboxylic acid N-(piperidin-4-yl)amide (AT7519, CAS 844442-38-2); 4-[2-methyl-1-(1-methylethyl)-1H-imidazol-5-yl]-N-[4-(methylsulfonyl)phenyl]-2-pyrimidinamine (AZD5438, CAS 602306-29-6); palbociclib (PD-0332991); and (2R,3R)-3-[[2-[[3[[S(R)]-S-cyclopropylsulfonimidoyl]-phenyl]amino]-5-(trifluoromethyl)-4-pyrimidinyl]oxy]-2-butanol (BAY 10000394).

p53-MDM2 inhibitors: (S)-1-(4-chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, (S)-5-(5-chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, [(4S,5R)-2-(4-tert-butyl-2-ethoxyphenyl)-4,5-bis(4-chlorophenyl)-4,5-dimethylimidazol-1-yl]-[4-(3-methylsulfonylpropyl)piperazin-1-yl]methanone (RG7112), 4-[[(2R,3S,4R,5S)-3-(3-chloro-2-fluorophenyl)-4-(4-chloro-2-fluorophenyl)-4-cyano-5-(2,2-dimethylpropyl)pyrrolidine-2-carbonyl]amino]-3-methoxybenzoic acid (RG7388), SAR299155, 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin yl)acetic acid (AMG232), {(3R,5R,6S)-5-(3-chlorophenyl)-6-(4-chlorophenyl)-1-[(2S,3S) hydroxy-3-pentanyl]-3-methyl-2-oxo-3-piperidinyl}acetic acid (AM-8553), (±)-4-[4,5-bis(4-chlorophenyl)-2-(2-isopropoxy-4-methoxy-phenyl)-4,5-dihydro-imidazole-1-carbonyl]-piperazin-2-one (Nutlin-3), 2-methyl-7-[phenyl(phenylamino)methyl]-8-quinolinol (NSC 66811), 1-N-[2-(1H-indol-3-yl)ethyl]-4-N-pyridin-4-ylbenzene-1,4-diamine (JNJ-26854165), 4-[4,5-bis(3,4-chlorophenyl)-2-(2-isopropoxy-4-methoxy-phenyl)-4,5-dihydro-imidazole carboxyl]-piperazin-2-one (Caylin-1), 4-[4,5-bis(4-trifluoromethyl-phenyl)-2-(2-isopropoxy-4-methoxy-phenyl)-4,5-dihydro-imidazole-1-carboxyl]-piperazin-2-one (Caylin-2), 5-[[3-dimethylamino)propyl]amino]-3,10-dimethylpyrimido[4,5-b]quinoline-2,4(3H,10H)-dione dihydrochloride (HLI373) and trans-4-iodo-4′-boranyl-chalcone (SC204072).

Mitogen-activated protein kinase (MEK) inhibitors: XL-518 (also known as GDC-0973, CAS No. 1029872-29-4, available from ACC Corp.); selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886, described in PCT Publication No. WO 2003/077914); 2-[(2-chloro-4-iodophenyl)amino]-N-(cyclopropylmethoxy)-3,4-difluoro-benzamide (also known as CI-1040 or PD184352 and described in PCT Publication No. WO 2000/035436); N-[(2R)-2,3-dihydroxypropoxy]-3,4-difluoro-2-[(2-fluoro-4-iodophenyl)amino]-benzamide (also known as PD0325901 and described in PCT Publication No. WO 2002/006213); 2,3-bis[amino[(2-aminophenyl)thio]methylene]-butanedinitrile (also known as U0126 and described in U.S. Pat. No. 2,779,780); N-[3,4-difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO 2007/014011); (3S,4R,5Z,8S,9S,11E)-14-(ethylamino)-8,9,16-trihydroxy-3,4-dimethyl-3,4,9; 19-tetrahydro-1H-2-benzoxacyclotetradecine-1,7(8H)-dione] (also known as E6201 and described in PCT Publication No. WO 2003/076424); 2′-amino-3′-methoxyflavone (also known as PD98059 available from Biaffin GmbH & Co., KG, Germany); (R)-3-(2,3-dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); trametinib dimethyl sulfoxide (GSK-1120212, CAS 1204531-25-80); 2-(2-fluoro-4-iodophenylamino)-N-(2-hydroxyethoxy)-1,5-dimethyl-6-oxo-1,6-dihydropyridine-3-carboxamide (AZD 8330); 3,4-difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655); and 5-[(4-bromo-2-fluorophenyl)amino]-4-fluoro-N-(2-hydroxyethoxy)-1-methyl-1H-benzimidazole-6-carboxamide (MEK162).

m-TOR inhibitors, for example, AZD2014, as described by Broutin et al. in Insights into Significance of Combined Inhibition of MEK and m-TOR Signalling Output in KRAS-mutant Non-Small-Cell Lung Cancer, British J. of Cancer (2016) 115, pp 549-552, which publication, along with its references, is incorporated herein by reference.

Hypomethylating agents (HMA), for example, decitabine and azacytidine, or a prodrug thereof, and other azanucleosides.

B-RAF inhibitors: regorafenib (BAY73-4506, CAS 755037-03-7); tuvizanib (AV951, CAS 475108-18-0); vemurafenib (ZELBORAF®, PLX-4032, CAS 918504-65-1); encorafenib (also known as LGX818); 1-methyl-5-[[2-[5-(trifluoromethyl)-1H-imidazol-2-yl]-4-pyridinyl]oxy]-N-[4-(trifluoromethyl)phenyl-1H-benzimidazol-2-amine (RAF265, CAS 927880-90-8); 5-[1-(2-hydroxyethyl)-3-(pyridin-4-yl)-1H-pyrazol-4-yl]-2,3-dihydroinden-1-one oxime (GDC-0879, CAS 905281-76-7); 5-[2-[4-[2-(dimethylamino)ethoxy]phenyl]-5-(4-pyridinyl)-1H-imidazol-4-yl]-2,3-dihydro-1H-inden-1-one oxime (GSK2118436 or SB590885); (+/−)-methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662), dabrafenib (TAFINLAR®), and N-(3-(5-chloro-1H-pyrrolo[2,3-b]pyridine-3-carbonyl)-2,4-difluorophenyl)propane-1-sulfonamide (also known as PLX4720).

ALK inhibitors: crizotinib (XALKORI®).

PIM kinase inhibitors:

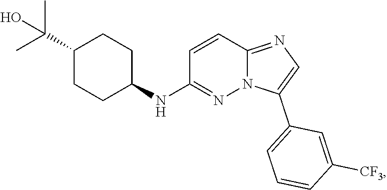 [SMILES: CC(C)(O)[C@H]1CC[C@H](Nc2ccc3ncc(-c4cccc(C(F)(F)F)c4)n3n2)CC1]

or a pharmaceutically acceptable salt thereof.

Proteasome inhibitors: bortezomib (VELCADE®), N-5-benzyloxycarbonyl-Ile-Glu(O-tert-butyl)-Ala-leucinal (PSI), carfilzomib and ixazomib (e.g., bortezomib), marizomib (NPI-0052), delanzomib (CEP-18770), 0-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912). A RNAi screen identified TNK1 as a potential modulator of proteasome inhibitor sensitivity in myeloma. Zhu et al., Blood (2011) 117 (14): 3847-3857. In some embodiments, a compound of the present disclosure (e.g., a compound of Formula I, or a subformula thereof, or a pharmaceutically acceptable salt of the foregoing) is administered in combination with a proteasome inhibitor described herein, e.g., for the treatment of multiple myeloma.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomeRASe inhibitor RFS 2000; difluoromethylornithine (DMFO).

Non-limiting examples of therapeutic agents that can be used in combinations with at least one compound of Formula I, II, III or any of the exemplified compounds of the invention, or one or more of any of these in the form of a pharmaceutically acceptable salt, are mTOR inhibitors. Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R, 23 S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4- azatricyclo[30.3.1.0^{4,9}] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-inner salt (SEQ ID NO: 1482) (SF1126, CAS 936487-67-1), and XL765.

Where desired, at least one compound of Formula I, II, III or any of the exemplified compounds of the invention, or any of these in the form of a pharmaceutically acceptable salt can be used in combination with commonly prescribed anti-cancer drugs (for example, but not limited to: anti-metabolites; DNA-fragmenting agents; DNA-crosslinking agents; intercalating agents; protein synthesis inhibitors; topoisomerase I and II poisons (for example, camptothecin, topotecan); microtubule-directed agents; kinase inhibitors; hormones; and hormone antagonists. Examples of some of these additional agents include, but are not limited to, Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BMW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

In some embodiments, at least one compound of Formula I, II, III or any of the exemplified compounds of the invention, or any of these in the form of a pharmaceutically acceptable salt is administered to a subject in need thereof in combination with a CDK9 inhibitor, such as Alvocidib. In a related embodiment, a pharmaceutically acceptable salt of a compound of structure (I) (e.g., a tartrate salt) is administered to a subject in need thereof in combination with a CDK9 inhibitor, such as Alvocidib. The administration may be before, concurrently or after administration of the CDK9 inhibitor. In some embodiments, a compound of Formula I, II, III or any of the exemplified compounds of the invention, or any of these in the form of a pharmaceutically acceptable salt is administered to a subject in need thereof in combination with a CDK9 inhibitor, such as Alvocidib for treatment of pancreatic cancer.

In some embodiments, the CDK inhibitor is a CDK2, CDK4, CDK6, CDK7, CDK8, CDK9, CDK10, and/or CDK11 inhibitor. In some embodiments, the CDK inhibitor is a CDK7, CDK9 inhibitor, or both. In some embodiments, the CDK inhibitor is dinaciclib (ACS Med. Chem. Lett. 2010 May 17; 1(5):204-8; Mol. Cancer Ther. 2010 August; 9(8):2344-53; Merck, Sharp and Dohme), AT7519 (J. Med. Chem. 2008 Aug. 28; 51(16):4986-99; Astex Pharmaceutical) or palbociclib (J. Med. Chem. 2005 Apr. 7; 48(7):2388-406; Pfizer). In certain embodiments, the CDK inhibitor is a CDK9 inhibitor, such as alvocidib. The alvocidib may be administered as the free bases, as a pharmaceutically acceptable salt or as a prodrug. In certain embodiments, the CDK9 inhibitor is alvocidib. in other embodiments, the CDK9 inhibitor is a pharmaceutically acceptable salt of alvocidib. In other embodiments, the CDK9 inhibitor is a prodrug of alvocidib. Prodrugs of alvocidib include those disclosed in WO 2016/187316, the full disclosure of which is hereby incorporated by reference in its entirety.

Various different cancers can be treated with the combination of at least one compound of Formulae I, II, III or any of the exemplified compounds of the invention, or any of these in the form of a pharmaceutically acceptable salt and CDK inhibitor. In some embodiments, the cancer is a hematologic cancer or solid tumor, for example any of the hematologic cancers or solid tumor cancers disclosed herein or known in the art.

In some specific embodiments, the cancer is a hematologic cancer, such as multiple myeloma, myelodysplastic syndrome (MDS), acute myelogenous leukemia (AML), acute lymphoblastic leukemia (ALL), acute lymphocytic leukemia, chronic lymphogenous leukemia, chronic lymphocytic leukemia (CLL), mantle cell lymphoma, diffuse large B-cell lymphoma, follicular lymphoma, or non-Hodgkin's lymphoma. In some specific embodiments, the hematologic cancer is CLL, SLL, or both. In some specific embodiments, the hematologic cancer is CLL. In some specific embodiments, the hematologic cancer is SLL.

In some other specific embodiments, the cancer treated by the combination of at least one NEK2 kinase inhibitor compound of the invention or the same in a pharmaceutically acceptable salt form and a CDK inhibitor is a solid tumor, such as a pancreatic, colon or lung cancer.

Embodiments further relate to a method of administering at least one compound of Formula I, II, III or any of the exemplified compounds of the invention, or any of these in the form of a pharmaceutically acceptable salt, to a subject in need thereof in combination with a BTK inhibitor (e.g., Ibrutinib) or a CDK9 inhibitor (e.g., Alvocidib) provided herein, in combination with radiation therapy for inhibiting abnormal cell growth or treating the hyperproliferative disorder in the mammal. Techniques for administering radiation therapy are known in the art, and these techniques can be used in the combination therapy described herein. The administration of at least one compound of Formula I, II, III or any of the exemplified compounds of the invention, or any of these in the form of a pharmaceutically acceptable salt in this combination therapy can be determined as described herein.

In one embodiment, at least one compound of Formula I, II, III or any of the exemplified compounds of the invention, or any of these in the form of a pharmaceutically acceptable salt is administered to a subject in need thereof in combination with an ATR inhibitor, such as AZD6738 or VX-970. In a related embodiment, at least one NEK2 inhibitor of the invention in the form of a pharmaceutical salt is administered to a subject in need thereof in combination with an ATR inhibitor, such as AZD6738 or VX-970. The administration may be before, concurrently or after administration of the ATR inhibitor. In one specific embodiment, at least one compound of Formula I, II, III or any of the exemplified compounds of the invention, or one or more of any of these in the form of a pharmaceutically acceptable salt is administered to a subject in need thereof in combination with an ATR inhibitor, such as AZD6738 or VX-970 for treatment of non-small cell lung cancer. In a related specific embodiment, at least one compound of Formula I, II, III or any of the exemplified compounds of the invention, in the form of a pharmaceutically acceptable salt is administered to a subject in need thereof in combination with an ATR inhibitor, such as AZD6738 or VX-970 for treatment of non-small cell lung cancer. In some of the foregoing embodiments, the salt is a tartrate salt. In some of the foregoing embodiments, the ATR inhibitor is AZD6738. In some of the foregoing embodiments, the ATR inhibitor is VX-970 or AZD6738. In some of the foregoing embodiments, the ATR inhibitor is a combination of AZD6738 and VX-970.

In some of the foregoing embodiments, the non-small cell lung cancer comprises TCGA lung adenocarcinoma, one or more LUAD tumors, TCGA lung squamous cell carcinoma, one or more LUSC tumors, one or more MDACC PROSPECT tumors, one or more MDACC BATTLE1 tumors, one or more BATTLE2 tumors, or combinations thereof. In some embodiments, the non-small cell lung cancer comprises TCGA LUAD tumors, for example, tumors enriched in ALK translocations. In some embodiments, the non-small cell lung cancer comprises TCGA LUAD tumors, for example, tumors comprising one or more EGFR mutations.

In one embodiment, at least one compound of Formula I, II, III or any of the exemplified compounds of the invention, or any of these in the form of a pharmaceutically acceptable salt, is administered to a subject in need thereof thereby sensitizing the subject to administration of an ATR inhibitor, such as AZD6738 or VX-970. In a related embodiment, a pharmaceutically acceptable salt of a compound of structure (I) (e.g., a tartrate salt) is administered to a subject in need thereof thereby sensitizing the subject to administration of an ATR inhibitor, such as AZD6738 or VX-970. In one specific embodiment, at least one compound of Formula I, II, III or any of the exemplified compounds of the invention, or any of these in the form of a pharmaceutically acceptable salt is administered to a subject in need thereof thereby sensitizing the subject to administration of an ATR inhibitor, such as AZD6738 or VX-970 for treatment of non-small cell lung cancer. In a related specific embodiment, at least one compound of Formula I, II, III, or any of the exemplified compounds of the invention or one or more of any of these in the form of a pharmaceutically-acceptable salt is administered to a subject in need thereof thereby sensitizing the subject to administration of an ATR inhibitor, such as AZD6738 or VX-970 for treatment of non-small cell lung cancer. In some of the foregoing embodiments, the ATR inhibitor is AZD6738. In some of the foregoing embodiments, the ATR inhibitor is VX-970. In some embodiments, the salt is a tartrate salt and the ATR inhibitor is AZD6738. In some embodiments, the salt is a tartrate salt and the ATR inhibitor is VX-970. In some of the foregoing embodiments, the ATR inhibitor is a combination of AZD6738 and VX-970.

In some embodiments, radiation therapy can be administered in combination with administration of at least one compound of Formula I, II, III or any of the exemplified compounds of the invention, or one or more of any of these in the form of a pharmaceutically acceptable salt. Exemplary radiation therapies include external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At211, I131, I125, Y90, Re186, Re188, Sm153, Bi212, P32, and radioactive isotopes of Lu). Suitable radiation sources for use as a cell conditioner of the present invention include both solids and liquids. By way of non-limiting example, the radiation source can be a radionuclide, such as I125, I131, Yb169, Ir192 as a solid source, I125 as a solid source, or other radionuclides that emit photons, beta particles, gamma radiation, or other therapeutic rays. The radioactive material can also be a fluid made from any solution of radionuclide(s), e.g., a solution of I125 or I131, or a radioactive fluid can be produced using a slurry of a suitable fluid containing small particles of solid radionuclides, such as Au198, Y90. Moreover, the radionuclide(s) can be embodied in a gel or radioactive micro spheres.

Without being limited by any theory, administration of at least one compound of Formula I, II, III or any of the exemplified compounds of the invention, or one or more of any of these in the form of a pharmaceutically acceptable salt can render abnormal cells more sensitive to treatment with radiation for purposes of killing and/or inhibiting the growth of such cells. Accordingly, some embodiments include a method for sensitizing abnormal cells in a mammal to treatment with radiation which comprises administering to the mammal an amount of at least one compound of Formula I, II, III or any of the exemplified compounds of the invention, or one or more of any of these in the form of a pharmaceutically acceptable salt, which amount is effective in sensitizing abnormal cells to treatment with radiation. The amount of one or more compounds of Formula I, II, III or any of the exemplified compounds of the invention, or one or more of any of these in the form of a pharmaceutically acceptable salt to be administered in this method can be determined according to the means for ascertaining effective amounts of such compounds and salts described herein.

One or more compounds of Formula I, II, III or any of the exemplified compounds of the invention, or one or more of any of these in the form of a pharmaceutically acceptable salt can also be used in combination with an amount of one or more substances selected from anti-angiogenesis agents, signal transduction inhibitors, antiproliferative agents, glycolysis inhibitors, or autophagy inhibitors.

Anti-angiogenesis agents include, for example, MMP-2 (matrix-metalloproteinase 2) inhibitors, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Embodiments of MMP-2 and MMP-9 inhibitors include those that have little or no activity inhibiting MMP-1. Other embodiments include those that selectively inhibit MMP-2 and/or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors useful in some embodiments are AG-3340, RO 323555, and RS 13-0830.

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

In other embodiments, agents useful in methods for combination therapy with at least one compound of Formula I, II, III or any of the exemplified compounds of the invention, or one or more of any of these in the form of a pharmaceutically acceptable salt include, but are not limited to: Erlotinib, Afatinib, Iressa, GDC0941, MLN1117, BYL719 (Alpelisib), BKM120 (Buparlisib), CYT387, GLPG0634, Baricitinib, Lestaurtinib, momelotinib, Pacritinib, Ruxolitinib, TG101348, Crizotinib, tivantinib, AMG337, cabozantinib, foretinib, onartuzumab, NVP-AEW541, Dasatinib, Ponatinib, saracatinib, bosutinib, trametinib, selumetinib, cobimetinib, PD0325901, RO5126766, Axitinib, Bevacizumab, Bostutinib, Cetuximab, Crizotinib, Fostamatinib, Gefitinib, Imatinib, Lapatinib, Lenvatinib, Ibrutinib, Nilotinib, Panitumumab, Pazopanib, Pegaptanib, Ranibizumab, Ruxolitinib, Sorafenib, Sunitinib, SU6656, Trastuzumab, Tofacitinib, Vandetanib, Vemurafenib, Irinotecan, Taxol, Docetaxel, Rapamycin or MLN0128.

In embodiments, at least one compound of Formula I, II, III or any of the compounds of the invention exemplified herein, or one or more of any of these in the form of a pharmaceutically acceptable salt is administered in combination with an epidermal growth factor receptor tyrosine kinase (EGFR) inhibitor, including EGFR inhibitors which are whole antibodies, for example cetuximab (Erbitux®). Examples of EGFR inhibitors include erlotinib, osimertinib, cetuximab, gefitinib, necitumumab, lapatinib, neratinib, panitumumab, vandetanib, and necitumumab. A combination of at least one compound of Formula I, II, III or any of the compounds of the invention exemplified herein, or one or more of any of these in the form of a pharmaceutically acceptable salt and an EGFR inhibitor may be useful, for example, in the treatment of cancers that are related to EGFR dysregulation, such as non-small-cell lung cancer (NSCLC), pancreatic cancer, breast cancer, and colon cancer. EGFR may be dysregulated, for example, due to activating mutations in exons 18, 19, 20, or 21. In particular embodiments, the EGFR inhibitor is erlotinib or osimertinib. In particular embodiments, a combination of at least one compound of Formula I, II, III or any of the compounds of the invention exemplified herein, or one or more of any of these in the form of a pharmaceutically acceptable salt and an EGFR inhibitor is used to treat EGFR-mutated NSCLC. In particular embodiments, the combination of at least one compound of Formula I, II, III or any of the compounds of the invention exemplified herein, or any one or more of these in the form of a pharmaceutically acceptable salt and an EGFR inhibitor is used to treat an EGFR inhibitor-resistant cancer.

In certain embodiments, at least one compound of Formula I, II, III or any of the compounds of the invention exemplified herein, or one or more of any of these in the form of a pharmaceutically acceptable salt is administered in combination with Erlotinib. In some embodiments, such a combination is used to treat pancreatic cancer. In other embodiments, such a combination is used to treat lung cancer. In further embodiments, the lung cancer is non-small cell lung cancer.

In certain embodiments, at least one compound of Formula I, II, III or any of the compounds of the invention exemplified herein, or one or more of any of these in the form of a pharmaceutically acceptable salt, is administered in combination with osmertinib. In some embodiments, such a combination is used to treat lung cancer. In further embodiments, the lung cancer has an EGFR mutation.

When used in combination therapy, administration of at least one compound of Formula I, II, III or any of the compounds of the invention exemplified herein, or one or more of any of these in the form of a pharmaceutically acceptable salt, is in combination with administration of a second agent. This administration in combination can include simultaneous administration of the two agents in the same dosage form, simultaneous administration in separate dosage forms, and separate contemporaneous administration. That is, at least one compound of Formula I, II, III or any of the compounds of the invention exemplified herein, or one or more of any of these in the form of a pharmaceutically acceptable salt, and any of the agents described above (e.g., Ibrutinib or Alvocidib) can be formulated together in the same dosage form and administered simultaneously. Alternatively, at least one compound of Formula I, II, III or any of the compounds of the invention exemplified herein, or one or more of any of these in the form of a pharmaceutically acceptable salt, and any of the agents described above (e.g., Ibrutinib or Alvocidib) can be simultaneously administered, wherein both the agents are present in separate pharmaceutical compositions. In another alternative, at least one compound of Formula I, II, III or any of the compounds of the invention exemplified herein, or one or more of any of these in the form of a pharmaceutically acceptable salt, can be administered just followed by and any of the agents described above, or vice versa. In some embodiments of the separate administration protocol, at least one compound of Formula I, II, III or any of the compounds of the invention exemplified herein, or one or more of any of these in the form of a pharmaceutically acceptable salt, and any of the agents described above are administered a few minutes apart, or a few hours apart, or a few days apart.

When administering at least one compound of Formula I, II, III or any of the compounds of the invention exemplified herein, or one or more of any of these in the form of a pharmaceutically acceptable salt, in any of the foregoing embodiments of the disclosure, said compound may also be administered simultaneously with, prior to, or after administration of one or more additional therapeutic agents. For example, said at least one compound can be administered and after a sufficient period of time a second therapeutic agent is administered. In such embodiments, the period of time between administering at least one compound of Formula I, II, III or any of the compounds of the invention exemplified herein, or one or more of any of these in the form of a pharmaceutically acceptable salt, and the second therapeutic agent may be referred to as a “treatment break.” In some embodiments, such a treatment break ranges from about 12 hours to about 48 hours. In some embodiments, such a treatment break ranges from about 18 to about 40 hours. In some embodiments, such a treatment break ranges from about 18 to about 36 hours. In some embodiments, such a treatment break ranges from about 24 to about 48 hours. One of ordinary skill in the art can derive an appropriate dosing schedule based on common techniques and knowledge.

In some embodiments, at least one compound of Formula I, II, III or any of the compounds of the invention exemplified herein, or one or more of any of these in the form of a pharmaceutically acceptable salt and a second therapeutic agent are administered sequentially. In some embodiments, there is a treatment break between administering at least one compound of Formula I, II, III or any of the compounds of the invention exemplified herein, or one or more of any of these in the form of a pharmaceutically acceptable salt and administering the second therapeutic agent.

### Salts, Pharmaceutical Compositions and Dosage Forms

When employed in treatment of a patient, the compounds described herein can be administered in unit dosage form (where a “unit dose” is a discrete amount of the pharmaceutical composition comprising a predetermined amount of the active ingredient) comprising a portion of a pharmaceutically acceptable composition (e.g., at least one compound of Formula I, II, III, or any of the exemplified compounds of the invention, or one or more of any of these in the form of a pharmaceutically acceptable salt, and at least one pharmaceutically acceptable excipient which may be part of a pharmaceutically acceptable carrier. A “pharmaceutically acceptable carrier” refers to media generally accepted in the art for the delivery of biologically active agents to animals, in particular, mammals, and in some embodiments, humans. Excipients which may comprise a pharmaceutically acceptable carrier, or any portion of a pharmaceutically acceptable composition, can include materials from the generally recognized as safe (GRAS) list, including solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like, and combinations thereof, as would be known to those skilled in the art (see, for example, Allen, L. V., Jr. et al., Remington: The Science and Practice of Pharmacy (2 Volumes), 22nd Edition, Pharmaceutical Press (2012). A pharmaceutically acceptable carrier, as the phrase is used herein, is distinguished from a “carrier” that forms a portion of a prodrug, as discussed above with regard to prodrug carriers.

These compositions can be prepared in a manner well known in the pharmaceutical art, and can be administered by a variety of routes, depending upon whether local or systemic treatment is desired and upon the area to be treated and depending upon the suitability of the compound itself for being adapted to a particular route of administration. Accordingly, routes of administration which may be suitable include topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions for topical administration may be made into unit dosage forms which include transdermal patches, ointments, lotions, creams, gels, drops, suppositories, sprays, liquids and powders. Pharmaceutical compositions may contain conventional pharmaceutical excipients, aqueous, powder or oily bases, thickeners and the like may be necessary or desirable, any or all of which may form part of a pharmaceutically acceptable carrier into which a compound of the invention, or a salt thereof, is incorporated. Coated condoms, gloves and the like may also be useful.

This invention also includes pharmaceutical compositions which contain, as the active ingredient, one or more of the compounds of Formula I, II, or III, or of any of the exemplified compounds of the invention, or one or more of any of these in the form of a salt, in combination with one or more excipients which may include a pharmaceutically acceptable carrier, and may be in any convenient form, for example, a solid, semi-solid, or liquid form or as a suspension in a carrier which is solid or liquid, and which may be suitable for injection, infusion or oral administration. For administering to patients, pharmaceutical compositions of the invention are usually prepared in individual dosage forms, for example, a lozenge, tablet, capsule, sachet, strip (soluble or otherwise), or in any other form which may be adapted to administration to a patient, Thus, pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a pharmaceutical composition, it will be appreciated that where the active compound is present in the form of a solid, the particle size can be adjusted by methods known in the art, for example, milling or agglomeration, to provide the appropriate particle size prior to or after combining with the other ingredients.

Some examples of suitable excipients include lactose, dextrose, sucrose, sorbitol, mannitol, starches, gum acacia, calcium phosphate, alginates, tragacanth gum, gelatin, calcium silicate, microcrystalline cellulose, polyvinylpyrrolidone, cellulose, water, syrup, and methyl cellulose. The pharmaceutical compositions can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 1 mg to about 1000 mg, for example, about 5 to about 1000 mg (1 g), more usually about 50 to about 500 mg, or from about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid unit dosage forms comprising pharmaceutically acceptable compositions, for example, tablets, capsules, infusion bag, the principal active ingredient may be mixed with one or more excipients to form a pharmaceutically acceptable composition suitable for incorporating into the unit dosage, for example, a tablet, filled capsule, or sealed IV bag. The pharmaceutically acceptable composition is then subdivided into unit dosage forms of the type described above containing from, for example, about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the pharmaceutical composition in an appropriate manner. In such an instance, the delivery device itself provides the unit dose of the pharmaceutical composition.

The amount of compound or composition administered to a patient, for example, the amount contained in a unit dose as described above, will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques or may be sterile filtered. Aqueous solutions can be packaged for use as is, or lyophilized, the lyophilized preparation being combined with a sterile aqueous pharmaceutically acceptable carrier prior to administration. In general, the pH of the compound preparations will be in a physiologically acceptable range, which is typically from about pH 3 to about pH 11. In some embodiments the pH is preferably from about pH 5 to about pH 9. In some embodiments the pH is preferably from about pH 7 to about pH 8.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Without being bound by tradition or theory, some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, the properties of the pharmaceutical composition, and its route of administration. Effective doses can be extrapolated from dose-response curves derived from in vitro or animal model test systems.

### Labeled Compounds and Assay Methods

Any formula given herein is intended to represent equally unlabeled forms of the compound as well as isotopically enriched (isotopically labeled) forms of the compounds. Isotopically labeled compounds have structures depicted by the formulas given herein but have been prepared such that one or more atoms in the structure have a greater than natural abundance of one or more isotopes of the selected atom. Examples of isotopes that can be incorporated into compounds of the present disclosure in excess of the naturally occurring abundance of these isotopes include isotopes of hydrogen, carbon, nitrogen, oxygen, phosphorous, fluorine, chlorine and iodine, for example ²H, ³H, ¹¹C, ¹³C, ¹⁴C, ¹⁵N, ¹⁸F, ³¹P, ³²P, ³⁵S, ³⁶Cl, ¹²³I, ¹²⁴I and ¹²⁵I, respectively. In some embodiments radionuclides for labeling compounds of the invention will be selected from ³H, ¹⁴C, ¹²⁵I, ³⁵S and ⁸²Br.

The present disclosure includes various isotopically labeled compounds as defined herein, for example those into which radioactive isotopes, such as ³H and ¹⁴C, or those into which non-radioactive isotopes, such as ²H and ¹³C are present at greater amounts than natural. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor,” as used herein, means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically labeled compounds of the present disclosure can generally be prepared by conventional techniques known to those skilled in the art or by processes disclosed in the schemes or in the examples and preparations described below (or analogous processes to those described hereinbelow), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

Accordingly, another aspect of the present invention relates to labeled compounds of the invention (radio-labeled, fluorescent-labeled, etc.) that would be useful not only in imaging techniques but also in assays, both in vitro and in vivo, for localizing and quantitating NEK2 in tissue samples, including human, and for identifying NEK2 ligands by inhibition binding of a labeled compound. Accordingly, the present invention includes NEK2 assays that contain such labeled compounds.

A labeled compound of the invention can be used in a screening assay to identify/evaluate compounds. For example, a newly synthesized or identified compound (i.e., test compound) which is labeled can be evaluated for its ability to bind NEK2 by monitoring its concentration variation when contacting with NEK2, through tracking of the labeling. For example, a test compound (labeled) can be evaluated for its ability to reduce binding of another compound which is known to bind to NEK2 (i.e., standard compound). Accordingly, the ability of a test compound to compete with the standard compound for binding to NEK2 directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Synthesis

Compounds of the invention, including salts, hydrates, and solvates thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Preparation of compounds of the invention can involve the protection and deprotection of various chemical groups. The need for protection and deprotection, and the selection of appropriate protecting groups can be readily determined by one skilled in the art. The chemistry of protecting groups can be found, for example, in T. W. Green and P. G. M. Wuts, Protective Groups in Organic Synthesis, 3^{rd}. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or C) infrared spectroscopy, spectrophotometry (e.g., UV-visible), or mass spectrometry, or by chromatography such as high performance liquid chromatography (HPLC) or thin layer chromatography. Compounds of the invention can be prepared according to numerous preparatory routes known in the literature. Example synthetic methods for preparing compounds of the invention are provided in the Scheme below.

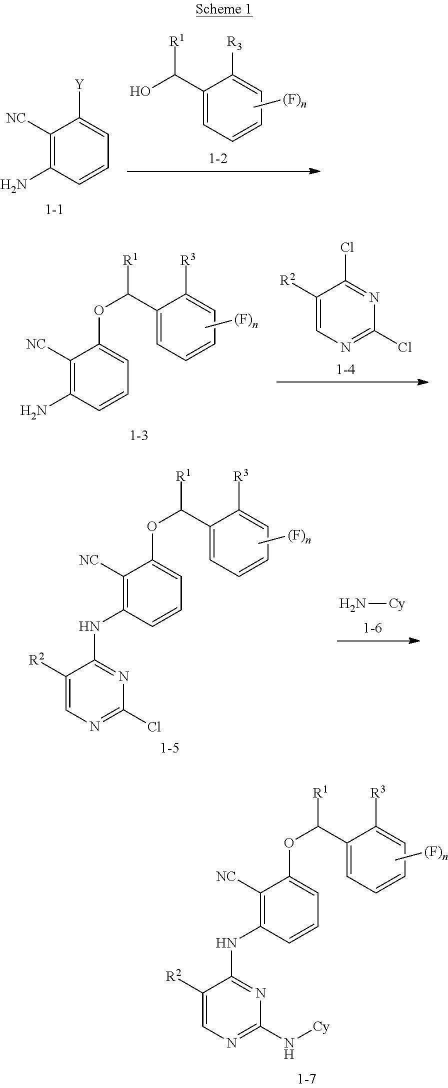

Scheme 1 provides an example synthesis of compounds of the invention. A 2-aminobenzonitrile compound 1-1 (wherein Y is a reactive halogen, for example F) can be treated with a phenylmethanol compound 1-2, optionally in the presence of heat, to form 2-amino-6-(benzyloxy)benzonitrile compound 1-3. Next, the pyrimidine moiety 1-4 is installed to form 1-5 using standard coupling conditions. The target compound 1-7 can be formed by treatment of 1-5 with an appropriate cyclic amine 1-6.

The present invention can further include synthetic methods for incorporating radioisotopes into compounds of the invention. Synthetic methods for incorporating radio-isotopes into organic compounds are well known in the art, and an ordinary skill in the art will readily recognize the methods applicable for the compounds of invention.

### Kits

The present invention also includes pharmaceutical kits useful, for example, in the treatment or prevention of NEK2-associated diseases or disorders, such as cancer, which include one or more containers containing a pharmaceutical composition comprising a therapeutically effective amount of a compound described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. The compounds of the Examples have been found to be NEK2 inhibitors according to at least one biological assay described herein.

### EXAMPLES

### Example 1

### 2-((5-chloro-2-((4-(4-(2-hydroxyethyl)piperazin-1-yl)phenyl)amino)pyrimidin-4-yl)amino)-6-((2-fluorobenzyl)oxy)benzonitrile (Compound 17)

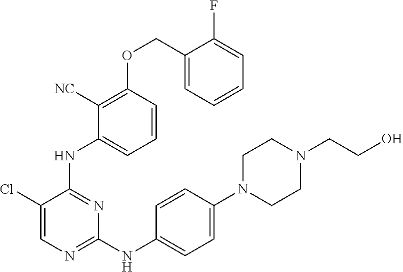 [SMILES: N#Cc1c(Nc2nc(Nc3ccc(N4CCN(CCO)CC4)cc3)ncc2Cl)cccc1OCc1ccccc1F]

The compound 2-((5-chloro-2-((4-(4-(2-hydroxyethyl)piperazin-1-yl)phenyl)amino)pyrimidin-4-yl)amino)-6-((2-fluorobenzyl)oxy)benzonitrile (Compound 17) was prepared according to the procedure set forth in Scheme 2.

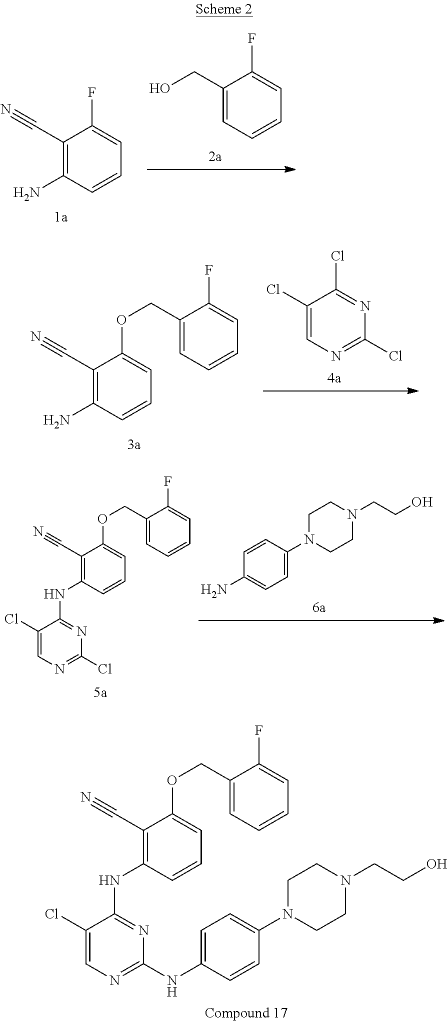

### Step 1: 2-amino-6-((2-fluorobenzyl) oxy) benzonitrile (3a)

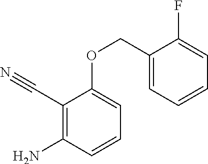 [SMILES: N#Cc1c(N)cccc1OCc1ccccc1F]

To a stirred solution of 2-amino-6-fluorobenzonitrile (1a; 10 g, 0.073 mol, 1.0 eq) and (2-fluorophenyl)methanol (2a; 9.26 g, 0.073 mol, 1.0 eq) in 1,4-dioxne (150 mL) was added cesium carbonate (47.45 g, 0.146 mol, 2.0 eq). The reaction mixture was heated to 100° C. for 40 h. After the completion of the reaction, it was filtered through celite bed. The filtrate was concentrated under reduced pressure. The crude compound obtained was purified by column chromatography to obtain 2-amino-6-((2-fluoro-benzyl) oxy) benzonitrile (3a; 10 g, 56%) as an off white solid.

### LCMS (UV):

### Column: Zorbax Eclipse Plus C18 (50×2.1 mm) 1.8 μm

Mobile Phase: A: 0.1% formic acid in water:acetonitrile (95:5)
Mobile phase: B: acetonitrile
Flow Rate: 0.8 mL/min
Retention time: 2.262 min; [M+H]⁺: 242.9

### Step 2: 2-((2,5-dichloropyrimidin-4-yl) amino)-6-((2-fluorobenzyl) oxy) benzonitrile (5a)

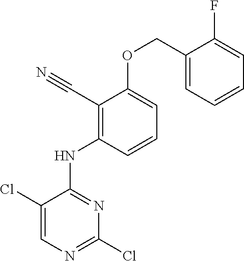 [SMILES: N#Cc1c(Nc2nc(Cl)ncc2Cl)cccc1OCc1ccccc1F]

To an ice-cold suspension of sodium hydride (2.46 g, 0.615 mol, 1.5 eq) in DMF (50 mL), a solution of 2-amino-6-((2-fluoro-benzyl) oxy) benzonitrile (3a, 10 g, 0.041 mol, 1 eq) in DMF (25 mL) was added dropwise. The resulting reaction mixture was stirred at same temperature for 0.5 h. Then a solution of 2,4,5-trichloropyrimidine (4a, 9.0 g, 0.049 mol, 1.2 eq) in DMF (25 mL) was added at 0° C. The reaction mixture was heated to 70° C. for 16h. After completion, the reaction mixture was quenched with water slowly. The solid precipitated was filtered and dried under vacuum. The crude compound was triturated with ethyl acetate (70 mL) and filtered to obtain 2-((2,5-dichloropyrimidin-4-yl) amino)-6-((2-fluorobenzyl) oxy) benzonitrile (5a, 6 g, 37.3%) as a pale brown solid.

### LCMS (UV):

### Column: Zorbax Eclipse Plus C18 (50×2.1 mm) 1.8 μm

Mobile Phase: A: 0.1% formic acid in water: acetonitrile (95:5)
Mobile phase: B: acetonitrile
Flow Rate: 0.8 mL/min
Retention time: 2.556 min; [M+H]⁺: 390.8

### Step 3: 2-((5-chloro-2-((4-(4-(2-hydroxyethyl) piperazin-1-yl) phenyl)amino)pyrimidin-4-yl)amino)-6-((2-fluorobenzyl)oxy)benzonitrile (Compound 17)

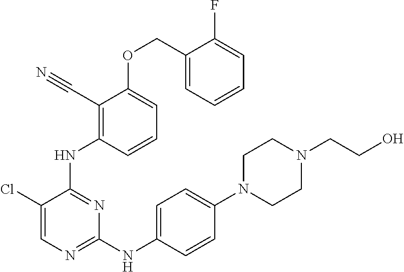 [SMILES: N#Cc1c(Nc2nc(Nc3ccc(N4CCN(CCO)CC4)cc3)ncc2Cl)cccc1OCc1ccccc1F]

To a stirred solution of 2-((2,5-dichloropyrimidin-4-yl)amino)-6-((2-fluorobenzyl)oxy) benzonitrile (5a, 0.2 g, 0.00051 mol, 1.0 eq) and 2-(4-(4-aminophenyl)piperazin-1-yl)ethan-1-ol (6a, 0.113 g, 0.00051 mol) in 2-methoxy ethanol (5 mL) added 4M HCl in dioxane (0.25 mL). The reaction mixture was heated to 100° C. for 16h in a sealed tube. After confirming the completion of reaction by LCMS, the reaction mixture was concentrated under reduced pressure. The crude compound was purified by RP PREP HPLC to afford the TFA salt of 2-((5-chloro-2-((4-(4-(2-hydroxyethyl)piperazin-1-yl)phenyl)amino)pyrimidin-4-yl)amino)-6-((2-fluorobenzyl)oxy)benzonitrile (Compound 17) as an off white solid (80 mg, 27.2%).

### LCMS (UV):

### Column: ZORBAX XDB C-18 (50×4.6 mm) 3.5 μm

Mobile Phase: A: 0.1% formic acid in water: acetonitrile (95:5)
Mobile phase: B: acetonitrile
Flow Rate: 1.5 mL/min
Retention time: 1.572 min. [M]⁺: 574.3

### HPLC:

### Column: X-Bridge C8(50×4.6) mm, 3.5 μm

Mobile Phase A: 0.1% trifluoroacetic acid in water
Mobile phase B: 0.1% trifluoroacetic acid in acetonitrile
Flow rate: 2.0 mL\min
Retention time: 3.186 min
Purity (max): 98.9%

¹H-NMR (400 MHz, DMSO-d6): δ 9.52 (s, 1H), 9.22 (m, 2H), 8.17 (s, 1H), 7.71 (t, J=8.40 Hz, 1H), 7.62 (t, J=1.60 Hz, 1H), 7.60-7.45 (m, 1H), 7.38 (d, J=8.80 Hz, 2H), 7.31-7.27 (m, 4H), 6.76 (d, J=8.40 Hz, 2H), 5.39 (s, 2H), 3.78 (t, J=5.60 Hz, 2H), 3.64-3.54 (m, 4H), 3.26 (d, J=4.80 Hz, 2H), 3.18 (d, J=10.80 Hz, 2H), and 2.94 (t, J=11.20 Hz, 2H).

### Example 2

### 2-((5-chloro-2-((4-(piperazin-1-yl)phenyl)amino)pyrimidin-4-yl)amino)-6-((2-fluorobenzyl)oxy)benzonitrile (Compound 16)

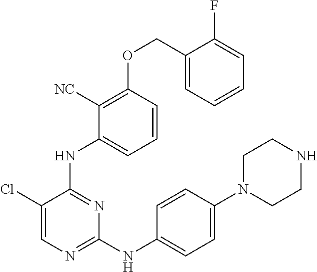 [SMILES: N#Cc1c(Nc2nc(Nc3ccc(N4CCNCC4)cc3)ncc2Cl)cccc1OCc1ccccc1F]

The compound 2-((5-chloro-2-((4-(piperazin-1-yl)phenyl)amino)pyrimidin-4-yl)amino)-6-((2-fluorobenzyl)oxy)benzonitrile (Compound 16) was synthesized according to the reaction shown in Scheme 3.

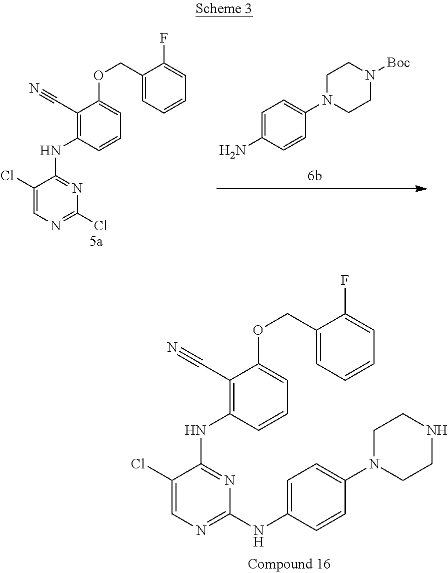 [SMILES: N#Cc1c(Nc2nc(Cl)ncc2Cl)cccc1OCc1ccccc1F ; CC(C)(C)OC(=O)N1CCN(c2ccc(N)cc2)CC1 ; N#Cc1c(Nc2nc(Nc3ccc(N4CCNCC4)cc3)ncc2Cl)cccc1OCc1ccccc1F]

To a stirred solution of 2-((2,5-dichloropyrimidin-4-yl)amino)-6-((2-fluorobenzyl)oxy)benzonitrile (5a, 1 g, 2.56 mol, 1.0 eq) and tert-butyl 4-(4-aminophenyl)-piperazine-1-carboxylate (6b, 0.71 g, 2.56 mol, 1 eq) in 2-methoxy ethanol (15 mL) added 4M HCl in dioxane (1.0 mL). The reaction mixture was heated to 100° C. for 16h in a sealed tube. After confirming the completion of reaction by TLC and LCMS, the reaction mixture was evaporated under reduced pressure. The crude compound was purified in reverse phase to afford the trifluoroacetic acid salt of 2-((5-chloro-2-((4-(piperazin-1-yl)phenyl)amino)pyrimidin-4-yl)amino)-6-((2-fluoro-benzyl)oxy)benzonitrile (Compound 16) as an off white solid (800 mg, 58%).

### LCMS (UV):

Column: XBRIDGE C8 (4.6×50 mm); 3.5 μm
Mobile Phase: A: 0.1% trifluoroacetic acid in water: acetonitrile (95:5)
Mobile phase: B: 0.1% trifluoroacetic acid in acetonitrile
Flow Rate: 1.5 mL/min
Retention time: 1.631 min. [M]⁺: 529.9

### HPLC:

### Column: X-Bridge C8(50×4.6) mm, 3.5 μm

Mobile Phase A: 0.1% trifluoroacetic acid in water
Mobile phase B: 0.1% trifluoroacetic acid in acetonitrile
Flow rate: 2.0 mL/min
Retention time: 3.207 min; Purity (max): 99.3%

¹H-NMR (400 MHz, DMSO-d6): δ 9.20 (m, 2H), 8.70 (s, 2H), 8.16 (s, 1H), 7.70-7.60 (m, 2H), 7.50-7.45 (m, 1H), 7.38 (d, J=8.40 Hz, 2H), 7.33-7.27 (m, 4H), 6.75 (d, J=8.80 Hz, 2H), 5.39 (s, 2H), and 3.20 (m, 8H).

### Example 3

### 2-((5-chloro-2-((4-(4-methylpiperazin-1-yl)phenyl)amino)pyrimidin-4-yl)amino)-6-(1-(2-fluorophenyl)ethoxy)benzonitrile (Compound 33)

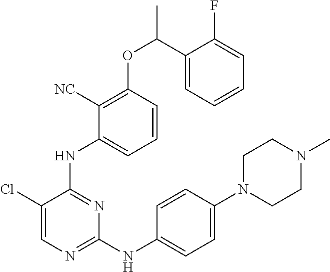 [SMILES: CC(Oc1cccc(Nc2nc(Nc3ccc(N4CCN(C)CC4)cc3)ncc2Cl)c1C#N)c1ccccc1F]

The compound 2-((5-chloro-2-((4-(4-methylpiperazin-1-yl)phenyl)amino)pyrimidin-4-yl)amino)-6-(1-(2-fluorophenyl)ethoxy)benzonitrile (Compound 00) was prepared according to the procedure set forth in Scheme 4.

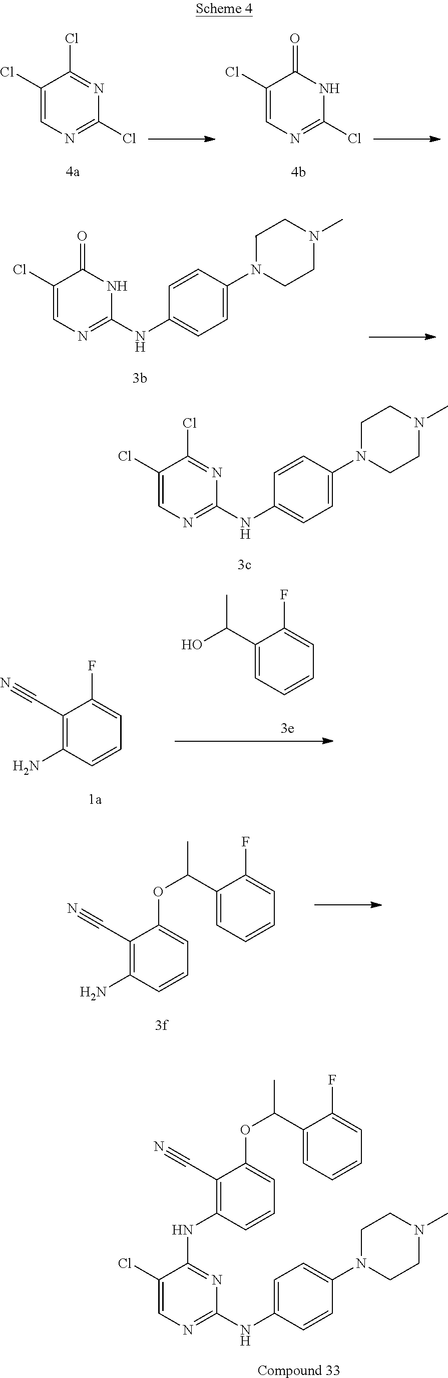

### Step 1: 2, 5-dichloropyrimidin-4(3H)-one (4b)

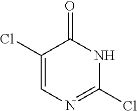

To an ice cold solution of 2, 4, 5-trichloropyrimidine (4a; 20 g, 0.11 mol, 1.0 eq) in THF (100 mL), 2N NaOH (240 mL) solution was added and the resulting reaction mixture was stirred at room temperature for 2h. The progress of the reaction was monitored by TLC. After completion of starting material, the reaction mixture was acidified with 1.5N HCl to ˜pH=4-6 and extracted with ethyl acetate. Combined organic part was dried over anhydrous sodium sulphate and concentrated under reduced pressure to afford 2,5-dichloropyrimidin-4(3H)-one (4b, 15 g, 82.17%) as an off white solid.

### LCMS (UV):

### Column: ZORBAX XDB C-18 (50×4.6 mm) 3.5 μm

Mobile Phase: A: 0.1% formic acid in water: acetonitrile (95:5)
Mobile phase: B: acetonitrile
Flow Rate: 1.5 mL/min
Retention time: 0.757 min. [M+H]⁺: 166.1

### Step 2: 5-chloro-2-((4-(4-methylpiperazin-1-yl) phenyl) amino) pyrimidin-4(3H)-one (3b)

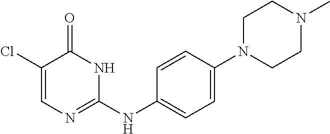 [SMILES: CN1CCN(c2ccc(Nc3ncc(Cl)c(=O)[nH]3)cc2)CC1]

To a stirred mixture of 2,5-dichloropyrimidin-4(3H)-one (4b; 10 g, 0.06 mol, 1.0 eq) and 4-(4-methylpiperazin-1-yl) aniline (11.57 g, 0.06 mol, 1.0 eq) in 2-methoxy ethanol (100 mL), 4M HCl in dioxane (10 mL) was added and the resulting mixture was heated to 100° C. for 16 h in a sealed tube. After confirming the completion of reaction by TLC, the reaction mixture was diluted with water and basified with saturated sodium bicarbonate solution up to pH=7-8. Then it was extracted with ethyl acetate (4×500 mL). The organic layer was washed with brine solution (250 mL×2), dried over sodium sulfate and concentrated under reduced pressure. The crude obtained was purified by column chromatography to obtain 5-chloro-2-((4-(4-methylpiperazin-1-yl)phenyl) amino)pyrimidin-4(3H)-one (3b, 6 g, 30%) as a yellow solid.

### LCMS (UV):

### Column: ZORBAX XDB C-18 (50×4.6 mm) 3.5 μm

Mobile Phase: A: 0.1% formic acid in water: acetonitrile (95:5)
Mobile phase: B: acetonitrile
Flow Rate: 1.5 mL/min
Retention time: 0.687 min. [M+H]⁺: 320.1.

### Step 3: 4, 5-dichloro-N-(4-(4-methylpiperazin-1-yl) phenyl) pyrimidin-2-amine (3c)

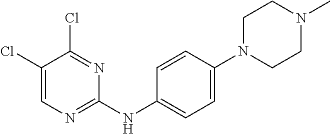 [SMILES: CN1CCN(c2ccc(Nc3ncc(Cl)c(Cl)n3)cc2)CC1]

A mixture of 5-chloro-2-((4-(4-methylpiperazin-1-yl)phenyl)amino)pyrimidin-4(3H)-one (3b; 6 g, 0.018 mol, 1.0 eq) and POCl₃ (60 mL) was heated to 85° C. for 10 h. The resulting reaction mixture was concentrated under reduced pressure and the residue was neutralized with saturated sodium bicarbonate solution to pH=7. Then it was extracted with 10% methanol in dichloromethane. The combined organic part was dried over anhydrous sodium sulphate and concentrated under reduced pressure to obtain 4, 5-dichloro-N-(4-(4-methylpiperazin-1-yl) phenyl) pyrimidin-2-amine (3c, 5.2 g, 82%) as a yellow solid.

### LCMS (UV):

### Column: ZORBAX XDB C-18 (50×4.6 mm) 3.5 μm

Mobile Phase: A: 0.1% formic acid in water: acetonitrile (95:5)
Mobile phase: B: acetonitrile
Flow Rate: 1.5 mL/min
Retention time: 1.431 min. [M]⁺: 338.1

### Step 4: 2-amino-6-(1-(2-fluorophenyl)ethoxy)benzonitrile (3f)

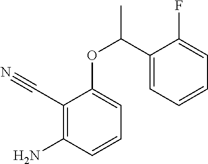 [SMILES: CC(Oc1cccc(N)c1C#N)c1ccccc1F]

To a stirred solution of 2-amino-6-fluorobenzonitrile (1a; 1 g, 0.0073 mol, 1.0 eq) and 1-(2-fluorophenyl)ethan-1-ol (3e; 1.030 g, 0.0073 mol, 1.0 eq) in 1, 4-dioxne (20 mL) was added cesium carbonate (4.745 g, 0.0146 mol, 2.0 eq). The reaction mixture was heated to 100° C. for 70 h. After the completion of the reaction, it was filtered through celite bed. The filtrate was concentrated under reduced pressure. The crude compound obtained was purified by column chromatography to obtain 2-amino-6-(1-(2-fluorophenyl)ethoxy)benzonitrile (3f, 250 mg, 13.29%) as an off white solid.

### LCMS (UV):

Column:)(BRIDGE C8 (4.6×50 mm); 3.5 μm
Mobile Phase: A: 0.1% trifluoroacetic acid in water: acetonitrile (95:5)
Mobile phase: B: 0.1% trifluoroacetic acid in acetonitrile
Flow Rate: 1.5 mL/min
Retention time: 2.405 min; [M+H]⁺: 257.1

### Step 5: 2-((5-chloro-2-((4-(4-methylpiperazin-1-yl)phenyl)amino)pyrimidin-4-yl)amino)-6-(1-(2-fluorophenyl)ethoxy)benzonitrile (Compound 00)

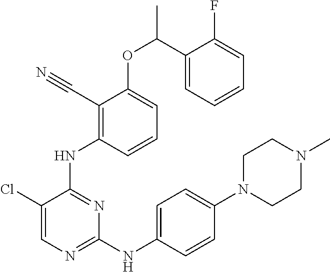 [SMILES: CC(Oc1cccc(Nc2nc(Nc3ccc(N4CCN(C)CC4)cc3)ncc2Cl)c1C#N)c1ccccc1F]

To a stirred solution of 2-amino-6-(1-(2-fluorophenyl)ethoxy)benzonitrile (3f; 0.25 g, 0.00097 mol, 1.0 eq) and 4,5-dichloro-N-(4-(4-methylpiperazin-1-yl)phenyl)pyrimidin-2-amine (3c; 0.33 g, 0.00097 mol, 1.0 eq) in 1,4-dioxane (15 mL), cesium carbonate (0.475 g, 0.00146 mol, 1.5 eq) was added. The resulting mixture was degassed with nitrogen gas for 20 minutes. Then XantPhos (56 mg, 0.000097 mol, 0.1 eq) and palladium (II) acetate (11 mg, 0.000048 mol, 0.05 eq) were added and heated to 100° C. for 16h in a sealed tube. After completion of starting material, reaction mass was filtered through celite bed. The filtrate was concentrated under reduced pressure. The crude compound obtained was purified by RP PREP HPLC to afford the TFA salt of 2-((5-chloro-2-((4-(4-methylpiperazin-1-yl)phenyl)amino)pyrimidin-4-yl)amino)-6-(1-(2-fluorophenyl)-ethoxy)-benzonitrile (Compound 00, 0.1 g, 18.5%) as an off white solid.

### LCMS (UV):

Column:)(BRIDGE C8 (4.6×50 mm); 3.5 μm
Mobile Phase: A: 0.1% trifluoroacetic acid in water: acetonitrile (95:5)
Mobile phase: B: 0.1% trifluoroacetic acid in acetonitrile
Flow Rate: 1.5 mL/min
Retention time: 1.848 min; [M]⁺: 558.2

### HPLC:

### Column: X-Bridge C8(50×4.6) mm, 3.5 μm

Mobile Phase A: 0.1% trifluoroacetic acid in water
Mobile phase B: 0.1% trifluoroacetic acid in acetonitrile
Flow rate: 2.0 mL\min
Retention time: 3.524 min; Purity (max): 98.52%

¹H-NMR (400 MHz, DMSO-d6): δ 9.61 (s, 1H), 9.22 (s, 1H), 9.14 (s, 1H), 8.16 (s, 1H), 7.60-7.50 (m, 2H), 7.42-7.36 (m, 3H), 7.29-7.21 (m, 3H), 7.04 (d, J=8.52 Hz, 1H), 6.74 (d, J=8.88 Hz, 2H), 6.00-5.96 (m, 1H), 3.63 (d, J=11.96 Hz, 2H), 3.51 (d, J=11.88 Hz, 2H), 3.2-3.11 (m, 2H), 2.82-2.87 (m, 5H), and 1.67 (d, J=6.32 Hz, 3H).

### Example 4

### 2-((5-chloro-2-((1-(2-(dimethylamino)ethyl)-1H-pyrazol-4-yl)amino)pyrimidin yl)amino)-6-((2-fluorobenzyl)oxy)benzonitrile (Compound 32)

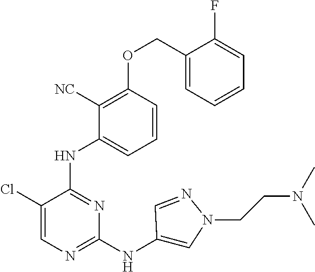 [SMILES: CN(C)CCn1cc(Nc2ncc(Cl)c(Nc3cccc(OCc4ccccc4F)c3C#N)n2)cn1]

The synthesis of the compound 2-((5-chloro-2-((1-(2-(dimethylamino)ethyl)-1H-pyrazol-4-yl)amino)pyrimidin-4-yl)amino)-6-((2-fluorobenzyl)oxy)benzonitrile (Compound 32) was prepared according to the procedure set forth in Scheme 5.

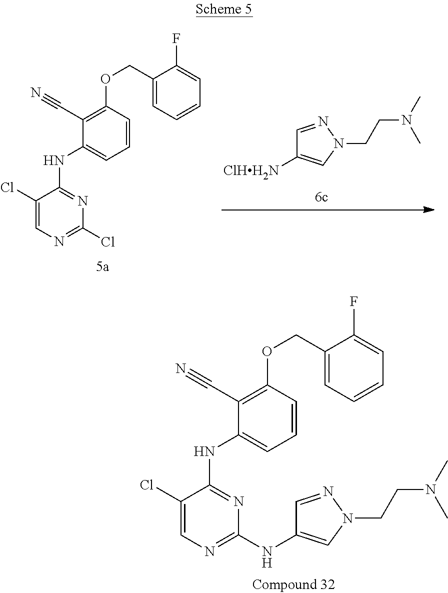

To a stirred solution of 2-((2,5-dichloropyrimidin-4-yl)amino)-6-((2-fluorobenzyl)oxy) benzonitrile (5a, 0.1 g, 0.00025 mol, 1.0 eq) and 1-(2-(dimethylamino)ethyl)-1H-pyrazol-4-amine hydrochloride (6c, 38 mg, 0.00025 mol, 1 eq) in 2-methoxy ethanol (10 mL) added 4M HCl in dioxane (0.1 mL). The reaction mixture was heated to 100° C. for 16h in a sealed tube. After confirming the completion of reaction by TLC and LCMS, the reaction mixture was evaporated under reduced pressure. The crude compound was purified by reverse phase preparatory HPLC to afford the trifluoroacetic acid salt of 2-((5-chloro-2-((1-(2-(dimethylamino)ethyl)-1H-pyrazol-4-yl)amino)pyrimidin-4-yl)-amino)-6-((2-fluorobenzyl)oxy)benzonitrile (Compound 32, 20 mg, 15.3%) as a brown solid.

### LCMS (UV):

### Column: ZORBAX XDB C-18 (50×4.6 mm) 3.5 μm

Mobile Phase: A: 0.1% formic acid in water: acetonitrile (95:5)
Mobile phase: B: acetonitrile
Flow Rate: 1.5 mL/min
Retention time: 1.539 min. [M]⁺: 507.1

### HPLC:

### Column: X-Bridge C8(50×4.6) mm, 3.5 μm

Mobile Phase A: 0.1% trifluoroacetic acid in water
Mobile phase B: 0.1% trifluoroacetic acid in acetonitrile
Flow rate: 2.0 mL\min
Retention time: 3.168 min; Purity (max): 95.84%

¹H-NMR (400 MHz, DMSO-d6): δ 9.25-9.43 (m, 3H), 8.16 (s, 1H), 7.74-7.60 (m, 2H), 7.50-7.45 (m, 1H), 7.36-7.25 (m, 5H), 5.38 (s, 2H), 3.54-3.48 (m, 4H), and 2.76 (s, 6H).

### Example 5

### 2-((5-chloro-2-((4-(4-methylpiperazin-1-yl)phenyl)amino)pyrimidin-4-yl)amino)-6-((2-fluorobenzyl)oxy)benzonitrile (Compound 18)

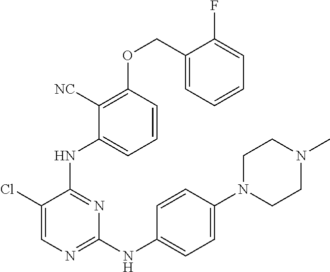 [SMILES: CN1CCN(c2ccc(Nc3ncc(Cl)c(Nc4cccc(OCc5ccccc5F)c4C#N)n3)cc2)CC1]

The synthesis of the compound 2-((5-chloro-2-((4-(4-methylpiperazin-1-yl)phenyl)amino)pyrimidin-4-yl)amino)-6-((2-fluorobenzyl)oxy)benzonitrile (Compound 18) was prepared according to the procedure summarized in Scheme 6.

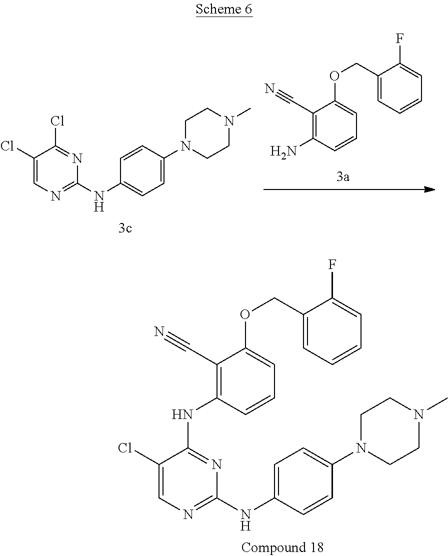

To a stirred solution of 2-amino-6-((2-fluorobenzyl)oxy)benzonitrile (3a; 3 g, 0.012 mol, 1.0 eq) and 4,5-dichloro-N-(4-(4-methylpiperazin-1-yl)phenyl)pyrimidin-2-amine (3c; 4.19 g, 0.012 mol, 1.0 eq) in 1,4-dioxane (50 mL), cesium carbonate (6.04 g, 0.018 mol, 1.5 eq) was added. The resulting mixture was degassed with nitrogen gas for 20 minutes. Then XantPhos (0.71 g, 0.0012 mol, 0.1 eq) and palladium (II) acetate (0.134 g, 0.0006 mol, 0.05 eq) were added and heated to 100° C. for 16h in a sealed tube. After completion of starting material, reaction mass was filtered through celite bed. The filtrate was concentrated under reduced pressure. The crude compound obtained was purified by column chromatography to obtain 2-((5-chloro-2-((4-(4-methyl-piperazin-1-yl)phenyl)amino)pyrimidin-4-yl)amino)-6-((2-fluorobenzyl) oxy) benzonitrile (Compound 18; 2 g, 29.6%) as an off white solid.

### LCMS (UV):

### Column: Zorbax Extend C18 (50×4.6 mm) 5 μm,

Mobile Phase: A: 10 mM Ammonium acetate in water

### Mobile Phase: B: Acetonitrile

Flow Rate: 1.2 mL/min
Retention time: 3.154 min; [M]⁺: 544.3

### HPLC:

### Column: Phenomenex Gemini C18 (150×4.6) mm, 3.0 μm

Mobile Phase A: 10 mM ammonium acetate in milli-q water
Mobile phase B: acetonitrile.
Flow rate: 1.0 mL\min
Retention time: 12.402 min;
Purity (max): 96.005%

¹H-NMR (400 MHz, DMSO-d6): δ 9.16 (s, 2H), 8.14 (s, 1H), 7.70-7.62 (m, 2H), 7.49-7.44 (m, 1H), 7.33-7.25 (m, 6H), 6.66 (d, J=8.40 Hz, 2H), 5.38 (s, 2H), 2.96 (m, 4H), 2.44 (m, 4H), and 2.23 (s, 3H).

### Example 6

### 2-((5-chloro-2-((4-morpholinophenyl)amino) pyrimidin-4-yl)amino)-6-((2 fluorobenzyl) oxy)benzonitrile (Compound 2)

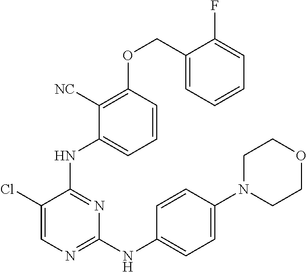 [SMILES: N#Cc1c(Nc2nc(Nc3ccc(N4CCOCC4)cc3)ncc2Cl)cccc1OCc1ccccc1F]

The compound 2-((5-chloro-2-((4-morpholinophenyl)amino)pyrimidin-4-yl)amino)-6-((2-fluorobenzyl)oxy)benzonitrile (Compound 2) was prepared according to the procedure summarized in Scheme 7.

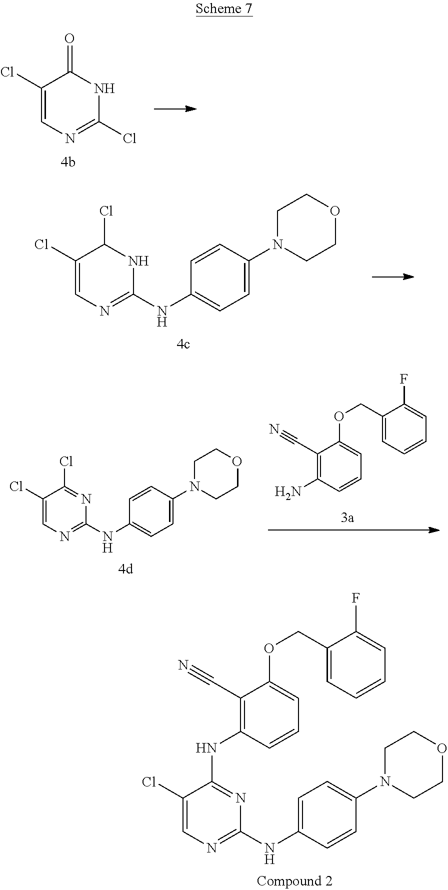 [SMILES: O=c1[nH]c(Cl)ncc1Cl ; ClC1=CN=C(Nc2ccc(N3CCOCC3)cc2)NC1Cl ; Clc1cnc(Nc2ccc(N3CCOCC3)cc2)nc1Cl ; N#Cc1c(N)cccc1OCc1ccccc1F ; N#Cc1c(Nc2nc(Nc3ccc(N4CCOCC4)cc3)ncc2Cl)cccc1OCc1ccccc1F]

### Step 1: 5-chloro-2-((4-morpholinophenyl)amino)pyrimidin-4-ol (4c)

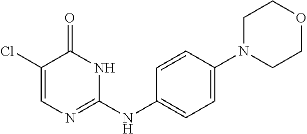 [SMILES: O=c1[nH]c(Nc2ccc(N3CCOCC3)cc2)ncc1Cl]

To a stirred mixture of 2,5-dichloropyrimidin-4(3H)-one (4b; 5 g, 0.03 mol, 1.0 eq) and 4-morpholinoaniline (5.34 g, 0.03 mol, 1.0 eq) in 2-methoxy ethanol (50 mL), 4M HCl in dioxane (5 mL) was added and the resulting mixture was heated to 100° C. for 16 h in a sealed tube. After confirming the completion of reaction by TLC, the reaction mixture was diluted with water and basified with saturated sodium bicarbonate solution up to pH=7-8. Then it was extracted with ethyl acetate (4×500 mL). The organic layer was washed with brine solution (250 mL×2), dried over sodium sulfate and concentrated under reduced pressure. The crude obtained was purified by column chromatography to obtain 5-chloro-2-((4-morpholinophenyl)amino)pyrimidin-4-ol (4c, 5 g, 53%) as a yellow solid.

### LCMS (UV):

### Column: ZORBAX XDB C-18 (50×4.6 mm) 3.5 μm

Mobile Phase: A: 0.1% formic acid in water: acetonitrile (95:5)
Mobile phase: B: acetonitrile
Flow Rate: 1.5 mL/min
Retention time: 1.017 min. [M+H]⁺: 307.1

### Step 2: 4,5-dichloro-N-(4-morphohnophenyl)pyrimidin-2-amine (4d)

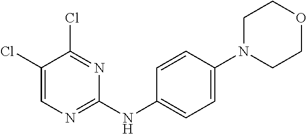 [SMILES: Clc1cnc(Nc2ccc(N3CCOCC3)cc2)nc1Cl]

A mixture of 5-chloro-2-((4-morpholinophenyl)amino)pyrimidin-4-ol (4c; 5 g, 0.016 mol, 1.0 eq) and POCl₃ (50 mL) was heated to 85° C. for 10 h. The resulting reaction mixture was concentrated under reduced pressure and the residue was neutralized with saturated sodium bicarbonate solution to pH=7. Then it was extracted with 10% methanol in dichloromethane. The combined organic part was dried over anhydrous sodium sulphate and concentrated under reduced pressure to obtain 4,5-dichloro-N-(4-morpholinophenyl)pyrimidin-2-amine (4d, 4.0 g, 75%) as a yellow solid.

### LCMS (UV):

### Column: ZORBAX XDB C-18 (50×4.6 mm) 3.5 μm

Mobile Phase: A: 0.1% formic acid in water: acetonitrile (95:5)
Mobile phase: B: acetonitrile
Flow Rate: 1.5 mL/min
Retention time: 2.216 min. [M]⁺: 325.0

### Step 3: 2-((5-chloro-2-((4-morphohnophenyl)amino)pyrimidin-4-yl)amino)-6-((2-fluorobenzyl)-oxy)benzonitrile (Compound 2)

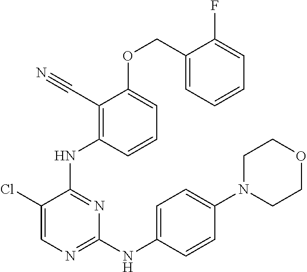 [SMILES: N#Cc1c(Nc2nc(Nc3ccc(N4CCOCC4)cc3)ncc2Cl)cccc1OCc1ccccc1F]

To a stirred solution of 2-amino-6-((2-fluorobenzyl)oxy)benzonitrile (3a; 2 g, 0.008 mol, 1.0 eq) and 4,5-dichloro-N-(4-morpholinophenyl)pyrimidin-2-amine (4d; 2.68 g, 0.008 mol, 1.0 eq) in 1,4-dioxane (50 mL), cesium carbonate (4.0 g, 0.012 mol, 1.5 eq) was added. The resulting mixture was degassed with nitrogen gas for 20 minutes. Then XantPhos (0.477 g, 0.00082 mol, 0.1 eq) and palladium (II) acetate (94 mg, 0.00041 mol, 0.05 eq) were added and heated to 100° C. for 16h in a sealed tube. After completion of starting material, reaction mass was filtered through celite bed. The filtrate was concentrated under reduced pressure. The crude compound was triturated with ethyl acetate (100 mL) to afford 2-((5-chloro-2-((4-morpholinophenyl)amino)-pyrimidin-4-yl)amino)-6-((2-fluorobenzyl)oxy)benzonitrile (Compound 2; 1.8 g, 41%) as an off white solid.

### LCMS (UV):

### Column: ZORBAX XDB C-18 (50×4.6 mm) 3.5 μm

Mobile Phase: A: 0.1% formic acid in water: acetonitrile (95:5)
Mobile phase: B: acetonitrile
Flow Rate: 1.5 mL/min
Retention time: 2.601 min; [M]⁺: 531.1

### HPLC:

### Column: X-Bridge C8(50×4.6) mm, 3.5 μm

Mobile Phase A: 0.1% trifluoroacetic acid in water
Mobile phase B: 0.1% trifluoroacetic acid in acetonitrile
Flow rate: 2.0 mL\min
Retention time: 3.882 min;
Purity (max): 96.17%.

¹H-NMR (400 MHz, DMSO-d6): δ 9.16 (d, J=4.48 Hz, 2H), 8.14 (s, 1H), 7.70 (t, J=8.28 Hz, 1H), 7.61 (t, J=7.76 Hz, 1H), 7.45 (t, J=5.72 Hz, 1H), 7.34-7.25 (m, 6H), 6.67 (d, J=8.56 Hz, 2H), 5.39 (s, 2H), 3.69 (t, J=4.92 Hz, 4H), and 2.92 (t, J=4.72 Hz, 4H).

The compounds in the following Table 1 include those exemplified above as well as additional compounds, which were made by methods analogous to those described in Examples 1-6 above.

TABLE 1

| Cmpd No. | Structure | Name |
|---|---|---|
| 1 | 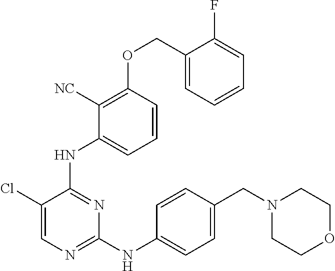 [SMILES: N#Cc1c(Nc2nc(Nc3ccc(CN4CCOCC4)cc3)ncc2Cl)cccc1OCc1ccccc1F] | 2-((5-chloro-2-((4- (morpholinomethyl)phenyl)amino) pyrimidin-4-yl)amino)-6-((2- fluorobenzyl)oxy)benzonitrile |
| 2 | 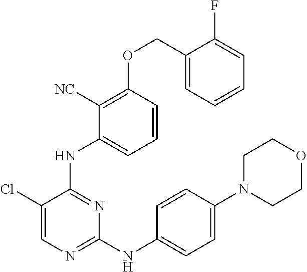 [SMILES: N#Cc1c(Nc2nc(Nc3ccc(N4CCOCC4)cc3)ncc2Cl)cccc1OCc1ccccc1F] | 2-((5-chloro-2-((4- morpholinophenyl)amino) pyrimidin-4-yl)amino)-6-((2- fluorobenzyl)oxy)benzonitrile |
| 3 | 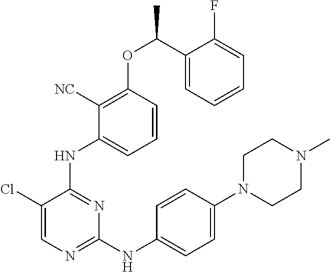 [SMILES: C[C@H](Oc1cccc(Nc2nc(Nc3ccc(N4CCN(C)CC4)cc3)ncc2Cl)c1C#N)c1ccccc1F] | (S)-2-((5-chloro-2-((4-(4-methylpiperazin- 1-yl)phenyl)amino) pyrimidin-4-yl)amino)-6-(1-(2- fluorophenyl)ethoxy)benzonitrile |
| 4 | 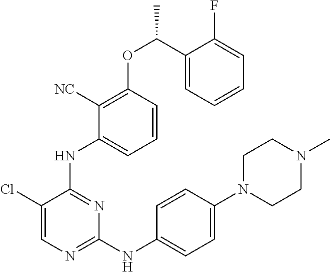 [SMILES: C[C@@H](Oc1cccc(Nc2nc(Nc3ccc(N4CCN(C)CC4)cc3)ncc2Cl)c1C#N)c1ccccc1F] | (R)-2-((5-chloro-2-((4-(4- methylpiperazin-1- yl)phenyl)amino)pyrimidin-4-yl)amino)- 6-(1-(2-fluorophenyl)ethoxy)benzonitrile |
| 5 | 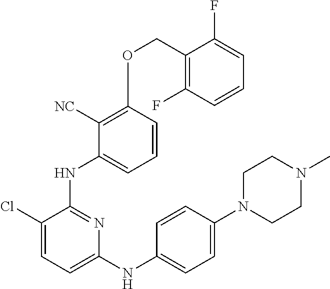 [SMILES: CN1CCN(c2ccc(Nc3ccc(Cl)c(Nc4cccc(OCc5c(F)cccc5F)c4C#N)n3)cc2)CC1] | 2-((5-chloro-2-((4-(4-methylpiperazin-1- yl)phenyl)amino) pyrimidin-4-yl)amino)-6-((2,6- difluorobenzyl)oxy)benzonitrile |
| 6 | 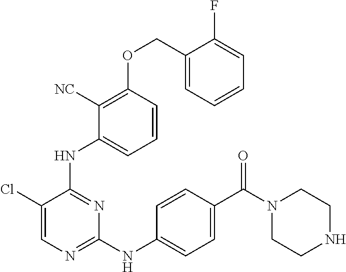 [SMILES: N#Cc1c(Nc2nc(Nc3ccc(C(=O)N4CCNCC4)cc3)ncc2Cl)cccc1OCc1ccccc1F] | 2-((5-chloro-2-((4-(piperazine-1- carbonyl)phenyl)amino)pyrimidin-4- yl)amino)-6-((2- fluorobenzyl)oxy)benzonitrile |
| 7 | 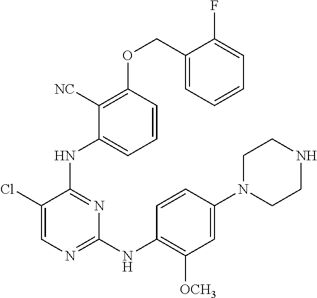 [SMILES: COc1cc(N2CCNCC2)ccc1Nc1ncc(Cl)c(Nc2cccc(OCc3ccccc3F)c2C#N)n1] | 2-((5-chloro-2-((2-methoxy-4-(piperazin- 1-yl)phenyl)amino) pyrimidin-4-yl)amino)-6-((2- fluorobenzyl)oxy)benzonitrile |
| 8 | 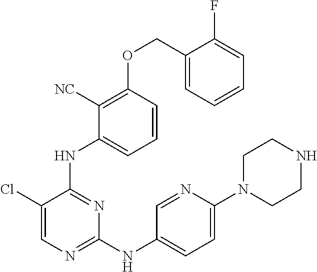 [SMILES: N#Cc1c(Nc2nc(Nc3ccc(N4CCNCC4)nc3)ncc2Cl)cccc1OCc1ccccc1F] | 2-((5-chloro-2-((6-(piperazin-1- yl)73yridine-3-yl)amino) pyrimidin-4-yl)amino)-6-((2- fluorobenzyl)oxy)benzonitrile |
| 9 | 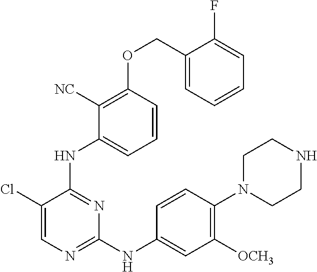 [SMILES: COc1cc(Nc2ncc(Cl)c(Nc3cccc(OCc4ccccc4F)c3C#N)n2)ccc1N1CCNCC1] | 2-((5-chloro-2-((3-methoxy-4-(piperazin- 1-yl)phenyl)amino)pyrimidin-4- yl)amino)-6-((2- fluorobenzyl)oxy)benzonitrile |
| 10 | 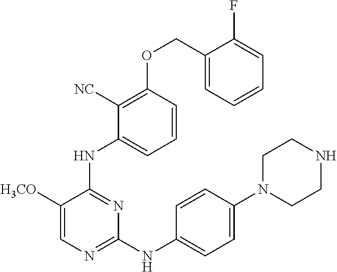 [SMILES: COc1cnc(Nc2ccc(N3CCNCC3)cc2)nc1Nc1cccc(OCc2ccccc2F)c1C#N] | 2-((2-fluorobenzyl)oxy)-6-((5-methoxy-2- ((4-(piperazin-1-yl) phenyl)amino)pyrimidin-4- yl)amino)benzonitrile |
| 11 | 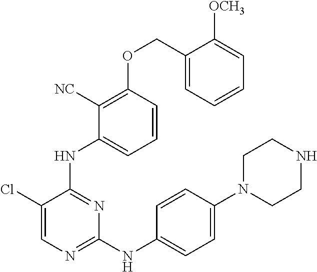 [SMILES: COc1ccccc1COc1cccc(Nc2nc(Nc3ccc(N4CCNCC4)cc3)ncc2Cl)c1C#N] | 2-((5-chloro-2-((4-(piperazin-1- yl)phenyl)amino) pyrimidin-4-yl)amino)-6-((2- methoxybenzyl)oxy)benzonitrile |
| 12 | 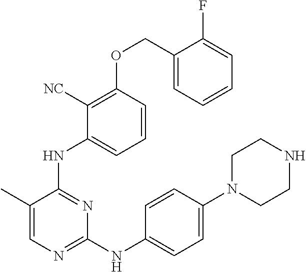 [SMILES: Cc1cnc(Nc2ccc(N3CCNCC3)cc2)nc1Nc1cccc(OCc2ccccc2F)c1C#N] | 2-((2-fluorobenzyl)oxy)-6-((5-methyl-2- ((4-(piperazin-1- yl)phenyl)amino)pyrimidin-4- yl)amino)benzonitrile |
| 13 | 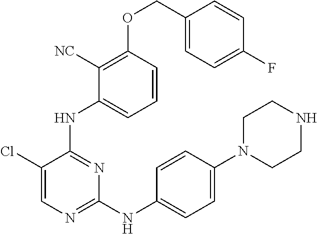 [SMILES: N#Cc1c(Nc2nc(Nc3ccc(N4CCNCC4)cc3)ncc2Cl)cccc1OCc1ccc(F)cc1] | 2-((5-chloro-2-((4-(piperazin-1- yl)phenyl)amino)pyrimidin-4-yl)amino)- 6-((4-fluorobenzyl)oxy)benzonitrile |
| 14 | 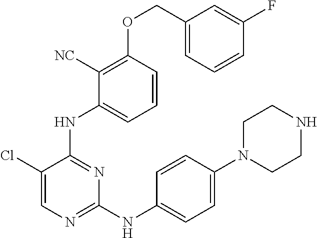 [SMILES: N#Cc1c(Nc2nc(Nc3ccc(N4CCNCC4)cc3)ncc2Cl)cccc1OCc1cccc(F)c1] | 2-((5-chloro-2-((4-(piperazin-1- yl)phenyl)amino)pyrimidin-4-yl)amino)- 6-((3-fluorobenzyl)oxy)benzonitrile |
| 15 | 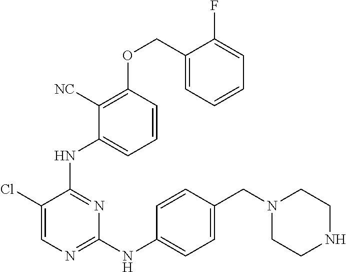 [SMILES: N#Cc1c(Nc2nc(Nc3ccc(CN4CCNCC4)cc3)ncc2Cl)cccc1OCc1ccccc1F] | 2-((5-chloro-2-((4-(piperazin-1- ylmethyl)phenyl)amino)pyrimidin-4- yl)amino)-6-((2- fluorobenzyl)oxy)benzonitrile |
| 16 | 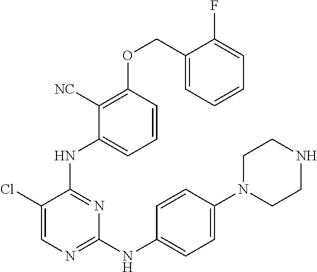 [SMILES: N#Cc1c(Nc2nc(Nc3ccc(N4CCNCC4)cc3)ncc2Cl)cccc1OCc1ccccc1F] | 2-((5-chloro-2-((4-(piperazin-1- yl)phenyl)amino)pyrimidin-4-yl)amino)- 6-((2-fluorobenzyl)oxy)benzonitrile |
| 17 | 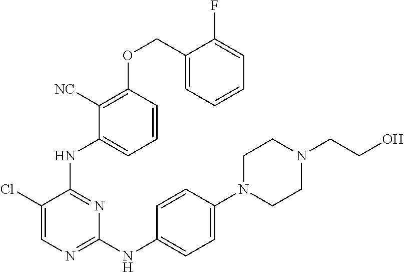 [SMILES: N#Cc1c(Nc2nc(Nc3ccc(N4CCN(CCO)CC4)cc3)ncc2Cl)cccc1OCc1ccccc1F] | 2-((5-chloro-2-((4-(4-(2- hydroxyethyl)piperazin-1- yl)phenyl)amino)pyrimidin-4-yl)amino)- 6-((2-fluorobenzyl)oxy)benzonitrile |
| 18 | 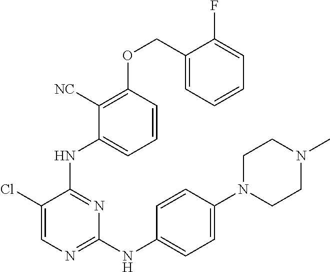 [SMILES: CN1CCN(c2ccc(Nc3ncc(Cl)c(Nc4cccc(OCc5ccccc5F)c4C#N)n3)cc2)CC1] | 2-((5-chloro-2-((4-(4-methylpiperazin-1- yl)phenyl)amino)pyrimidin-4-yl)amino)- 6-((2-fluorobenzyl)oxy)benzonitrile |
| 19 | 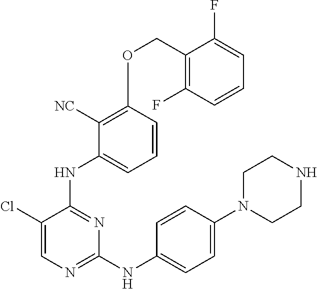 [SMILES: N#Cc1c(Nc2nc(Nc3ccc(N4CCNCC4)cc3)ncc2Cl)cccc1OCc1c(F)cccc1F] | 2-((5-chloro-2-((4-(piperazin-1- yl)phenyl)amino)pyrimidin-4-yl)amino)- 6-((2,6-difluorobenzyl)oxy)benzonitrile |
| 20 | 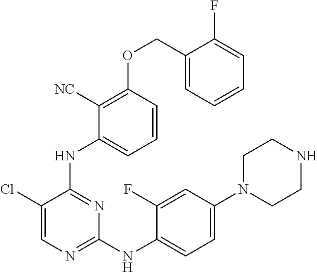 [SMILES: N#Cc1c(Nc2nc(Nc3ccc(N4CCNCC4)cc3F)ncc2Cl)cccc1OCc1ccccc1F] | 2-((5-chloro-2-((2-fluoro-4-(piperazin-1- yl)phenyl)amino)pyrimidin-4-yl)amino)- 6-((2-fluorobenzyl)oxy)benzonitrile |
| 21 | 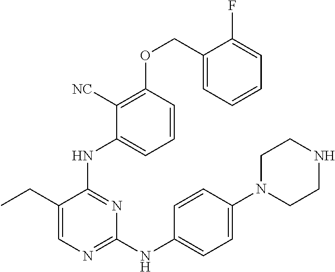 [SMILES: CCc1cnc(Nc2ccc(N3CCNCC3)cc2)nc1Nc1cccc(OCc2ccccc2F)c1C#N] | 2-((5-ethyl-2-((4-(piperazin-1- yl)phenyl)amino)pyrimidin-4-yl)amino)- 6-((2-fluorobenzyl)oxy)benzonitrile |
| 22 | 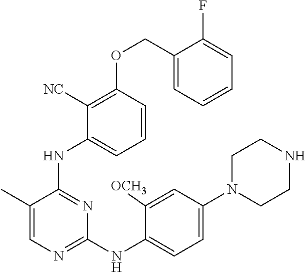 [SMILES: COc1cc(N2CCNCC2)ccc1Nc1ncc(C)c(Nc2cccc(OCc3ccccc3F)c2C#N)n1] | 2-((2-fluorobenzyl)oxy)-6-((2-((2- methoxy-4-(piperazin-1- yl)phenyl)amino)-5-methylpyrimidin-4- yl)amino)benzonitrile |
| 23 | 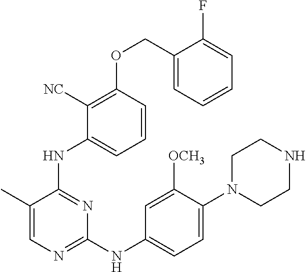 [SMILES: COc1cc(Nc2ncc(C)c(Nc3cccc(OCc4ccccc4F)c3C#N)n2)ccc1N1CCNCC1] | 2-((2-fluorobenzyl)oxy)-6-((2-((3- methoxy-4-(piperazin-1- yl)phenyl)amino)-5-methylpyrimidin-4- yl)amino)benzonitrile |
| 24 | 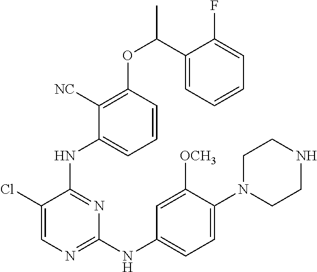 [SMILES: COc1cc(Nc2ncc(Cl)c(Nc3cccc(OC(C)c4ccccc4F)c3C#N)n2)ccc1N1CCNCC1] | 2-((5-chloro-2-((3-methoxy-4-(piperazin- 1-yl)phenyl)amino)pyrimidin-4- yl)amino)-6-(1-(2- fluorophenyl)ethoxy)benzonitrile |
| 25 | 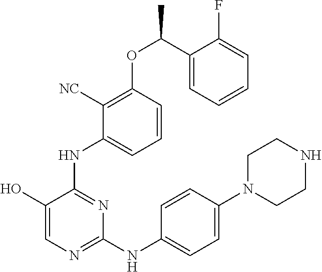 [SMILES: C[C@H](Oc1cccc(Nc2nc(Nc3ccc(N4CCNCC4)cc3)ncc2O)c1C#N)c1ccccc1F] | (S)-2-(1-(2-fluorophenyl)ethoxy)-6-((5- hydroxy-2-((4-(piperazin-1- yl)phenyl)amino)pyrimidin-4- yl)amino)benzonitrile |
| 26 | 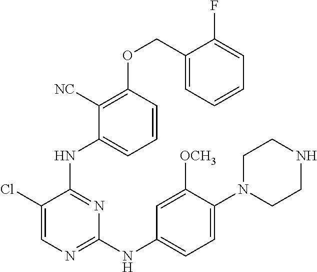 [SMILES: COc1cc(Nc2ncc(Cl)c(Nc3cccc(OCc4ccccc4F)c3C#N)n2)ccc1N1CCNCC1] | 2-((5-chloro-2-((3-methoxy-4-(piperazin- 1-yl)phenyl)amino)pyrimidin-4- yl)amino)-6-((2- fluorobenzyl)oxy)benzonitrile |
| 27 | 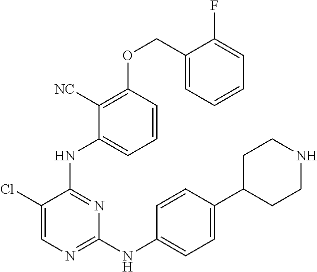 [SMILES: N#Cc1c(Nc2nc(Nc3ccc(C4CCNCC4)cc3)ncc2Cl)cccc1OCc1ccccc1F] | 2-((5-chloro-2-((4-(piperidin-4- yl)phenyl)amino)pyrimidin-4-yl)amino)- 6-((2-fluorobenzyl)oxy)benzonitrile |
| 28 | 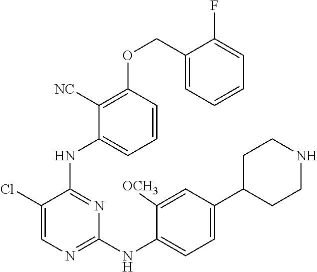 [SMILES: COc1cc(C2CCNCC2)ccc1Nc1ncc(Cl)c(Nc2cccc(OCc3ccccc3F)c2C#N)n1] | 2-((5-chloro-2-((2-methoxy-4-(piperidin- 4-yl)phenyl)amino)pyrimidin-4- yl)amino)-6-((2- fluorobenzyl)oxy)benzonitrile |
| 29 | 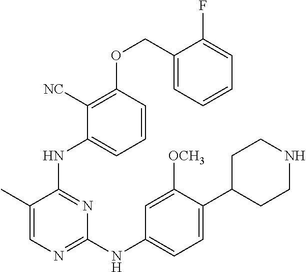 [SMILES: COc1cc(Nc2ncc(C)c(Nc3cccc(OCc4ccccc4F)c3C#N)n2)ccc1C1CCNCC1] | 2-((2-fluorobenzyl)oxy)-6-((2-((3- methoxy-4-(piperidin-4-yl)phenyl)amino)- 5-methylpyrimidin-4- yl)amino)benzonitrile |
| 30 | 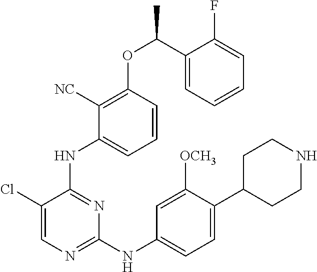 [SMILES: COc1cc(Nc2ncc(Cl)c(Nc3cccc(O[C@@H](C)c4ccccc4F)c3C#N)n2)ccc1C1CCNCC1] | (S)-2-((5-chloro-2-((3-methoxy-4- (piperidin-4-yl)phenyl)amino)pyrimidin- 4-yl)amino)-6-(1-(2- fluorophenyl)ethoxy)benzonitrile |
| 31 | 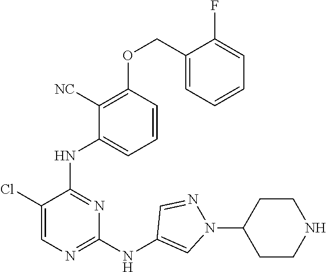 [SMILES: N#Cc1c(Nc2nc(Nc3cnn(C4CCNCC4)c3)ncc2Cl)cccc1OCc1ccccc1F] | 2-((5-chloro-2-((1-(piperidin-4-yl)-1H- pyrazol-4-yl)amino)pyrimidin-4- yl)amino)-6-((2- fluorobenzyl)oxy)benzonitrile |
| 32 | 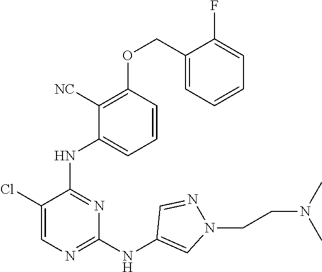 [SMILES: CN(C)CCn1cc(Nc2ncc(Cl)c(Nc3cccc(OCc4ccccc4F)c3C#N)n2)cn1] | 2-((5-chloro-2-((1-(2- (dimethylamino)ethyl)-1H-pyrazol-4- yl)amino)pyrimidin-4-yl)amino)-6-((2- fluorobenzyl)oxy)benzonitrile |
| 33 | 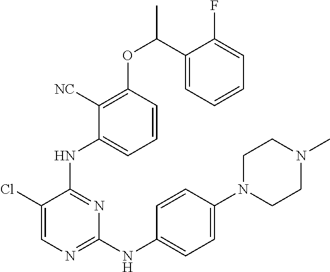 [SMILES: CC(Oc1cccc(Nc2nc(Nc3ccc(N4CCN(C)CC4)cc3)ncc2Cl)c1C#N)c1ccccc1F] | 2-((5-chloro-2-((4-(4-methylpiperazin-1- yl)phenyl)amino)pyrimidin-4-yl)amino)- 6-(1-(2-fluorophenyl)ethoxy)benzonitrile |

The compounds of Table 2 below, were prepared for comparison with compounds of the invention using methodologies similar to those described above. These compounds were subjected to various of the assays described herein with the results shown in Table 2.

TABLE 2
Comparator Compounds

| Cmpd. | | | | Aurora A |
|---|---|---|---|---|
| No. | Structure | Name | NEK2 Activity | Activity |
| CF-01 | 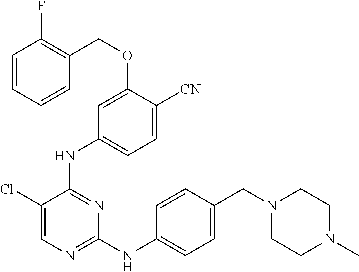 [SMILES: CN1CCN(Cc2ccc(Nc3ncc(Cl)c(Nc4ccc(C#N)c(OCc5ccccc5F)c4)n3)cc2)CC1] | 4-((5-chloro-2-((4-((4- methylpiperazin-1- yl)methyl)phenyl)- amino)-pyrimidin-4- yl)amino)-2-((2- fluorobenzyl)- oxy)benzonitrile | HotSpot ™ Assay >1 micromolar NanoBRET ™ assay 264 nM | 796 nM |
| CF-02 | 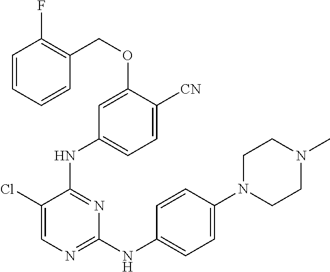 [SMILES: CN1CCN(c2ccc(Nc3ncc(Cl)c(Nc4ccc(C#N)c(OCc5ccccc5F)c4)n3)cc2)CC1] | 4-((5-chloro-2-((4-(4- methylpiperazin-1- yl)phenyl)-amino)- pyrimidin-4-yl)amino)- 2-((2-fluorobenzyl)- oxy)benzonitrile | HotSpot ™ Assay 264 nM | 313 nM |
| CF-03 | 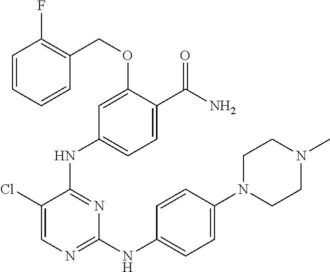 [SMILES: CN1CCN(c2ccc(Nc3ncc(Cl)c(Nc4ccc(C(N)=O)c(OCc5ccccc5F)c4)n3)cc2)CC1] | 4-((5-chloro-2-((4-(4- methylpiperazin-1- yl)phenyl)amino)- pyrimidin-4-yl)-amino)- 2-((2-fluorobenzyl) oxy)benzamide | HotSpot ™ Assay ND | 0.1 nM |

As the results shown in Table 2 indicate, minor structural and substituent differences in portions of the aminopyrimidinylaminobenzonitrile compounds can profoundly, and unpredictably, negatively impact the performance of these compounds with regard to suitable activity or selectivity for use as NEK2 inhibitors. As described herein, compounds of the invention, as reflected in the example compounds and compounds of Formulae I, II and III, and the activity data below, all display both sufficient or superior activity in NEK2 inhibition assays and sufficiently superior selectivity with respect to other kinases, for example, Aurora A, to be useful as NEK2 inhibitors.

### Assays Used to Determine Compound Activity and Selectivity

### Example A

### HotSpot™ Kinase Assay

In this example, the compounds described herein were tested for their inhibitory activity against NEK2 and/or Aurora A using the HotSpot™ Kinase Assay (Reaction Biology Corp.). The assay was performed according to the manufacturer's protocol, as detailed below. The NEK2 kinase (Accession No. NP-002488) and Aurora A kinase (Accession No. NP-940839) for use in these assays was sourced from ThermoFisher Scientific (Invitrogen).

The base Reaction Buffer used in this experiment contained the following: 20 mM Hepes (pH 7.5), 10 mM MgCl₂, 1 mM EGTA, 0.02% Brij™-35, 0.02 mg/ml BSA, 0.1 mM Na₃VO₄, 2 mM DTT, and 1% DMSO. The required cofactors were added individually to each kinase reaction.

Testing compounds were dissolved in 100% DMSO to a specific concentration. The serial dilution was conducted by Integra Viaflo Assist in DMSO.

The substrate was prepared in freshly prepared Reaction Buffer. The required cofactors were delivered to the substrate solution described above. NEK2 kinase or Aurora A kinase was delivered to the substrate solution and gently mixed. Next, the compounds in 100% DMSO were transferred to the kinase reaction mixture by Acoustic technology (Echo550; nanoliter range) and incubated for 20 min at room temperature. ³³P-ATP (Specific activity 10 μCi/μl) was delivered into the reaction mixture to initiate the reaction. The reaction was incubated for 2 hours at room temperature. Radioactivity was then detected by the filter-binding method. Kinase activity data were expressed as the percent remaining kinase activity in test samples compared to vehicle (dimethyl sulfoxide) reactions. IC₅₀ values and curve fits were obtained using Prism (GraphPad Software).

### Example B

### NanoBRET™ Target Engagement Assay for NEK2

In this example, the compounds described herein were tested for their inhibitory activity against NEK2 using a model of in vivo activity by the NanoBRET™ Target Engagement Intracellular Kinase Assay (Reaction Biology Corp.). The assay was performed according to the manufacturer's protocol, as detailed below.

### Step 1: Transient Transfection of HEK293 Cells NEK-2 NanoLuc® Fusion Vector DNA:

HEK293 cells were cultivated (70-80% confluence) prior to the assay. The sample was trypsinized and HEK293 cells were collected.

To prepare lipid: DNA complexes, a 10 μg/ml solution of DNA in Opti-MEM without serum was prepared that consisted of the following ratios of carrier DNA and DNA encoding NanoLuc® fusion: 9.0 μg/ml of Transfection Carrier DNA, 1.0 μg/ml of NanoLuc fusion vector DNA and 1 ml of Opti-MEM without phenol red. The solution was mixed thoroughly. 30 μl of FuGENE HD Transfection Reagent was added into each milliliter of DNA mixture to form lipid: DNA complex. The sample was mixed by inversion 10 times and then incubated at ambient temperature for 20 minutes to allow complexes to form. In a sterile, conical tube, 1 part of lipid:DNA complex was mixed with 20 parts of HEK293 cells in suspension. The resulting sample was mixed gently by inversion 5 times. The cell and lipid: DNA complex was dispensed into a sterile tissue culture dish and incubated for 22-24 hours.

### Step 2. Addition of Test Compounds:

Each test compound is delivered from the compound source plate to the wells of 384-well white NBS plate by Echo 550.

Step 3. Preparation of Cells with NanoBRET™ Tracer K5 Reagent:

The medium was removed from the dish with the transfected HEK293 cells via aspiration, trypsinized and cells were allowed to dissociate from the dish. Trypsin was neutralized using medium containing serum and centrifuged at 200×g for 5 minutes to pellet the cells. The cell density was adjusted to 2×10⁵ cells/ml in Opti-MEM without phenol red.

The Complete 20× NanoBRET™ Tracer K5 Reagent with Tracer Dilution Buffer was prepared according to the manufacturer's instructions. One part of Complete 20× NanoBRET™ Tracer Reagent was added to 20 parts of cells in the tube. The sample was mixed gently by inversion 10 times. Next, the cell suspension was dispensed into white, 384-well NBS plates with a final tracer K5 concentration of 2 μM. The plate was incubated at 37° C., 5% CO₂ for 1 hour. A separate set of samples without tracer were prepared for background correction steps.

### Step 4: NanoBRET™ Assay:

The plate was removed from the incubator and equilibrated to room temperature for 15 minutes. The 3× Complete Substrate plus Inhibitor Solution in Assay Medium (Opti-MEMR I Reduced Serum Medium, no phenol red) was prepared just before measuring BRET. The 3× Complete Substrate plus Inhibitor Solution was added to each well of the 384-well plate and incubated for 2-3 minutes at room temperature. The donor emission wavelength (460 nm) and acceptor emission wavelength (600 nm) were measured using the Envision 2104 plate reader.

### Step 5. Determination of BRET Ratio:

To generate raw BRET ratio values, the acceptor emission value (600 nm) was divided by the donor emission value (460 nm) for each sample. To correct for background, the BRET ratio in the absence of tracer (average of no-tracer control samples) was subtracted from the BRET ratio of each sample.

BRET Ratio=[(Acceptor sample÷Donor sample)−(Acceptor no-tracer control÷Donor no-tracer control)]

The in vitro assay results described in Assay Examples A, B and C are shown in Table 3; Compound (Cmpd.) No. is with reference to Cmpd. No. in Table 1.

TABLE 3
Assay Results For Selected Compounds of the Invention

| | | | NanoBRET ™ |
|---|---|---|---|
| | NEK2 IC₅₀ | Aurora A IC₅₀ | IC₅₀ |
| | +++≤ 5 nM | AAA ≤ 0.1 mM | BBB ≤ 50 nM |
| | 5 nM <++≤ | 0.1 mM < AA ≤ | 50 nM < BB ≤ |
| Cmpd | 15 nM +> | 1 mM | 150 nM |
| No. | 15 nM | A > 1 mM | B > 150 nM |
| 1 | + | AAA | BBB |
| 2 | +++ | A | B |
| 3 | +++ | AAA | BBB |
| 4 | + | AA | B |
| 5 | +++ | AAA | BBB |
| 6 | + | AAA | B |
| 7 | +++ | AA | B |
| 8 | ++ | NA | B |
| 9 | +++ | AAA | BBB |
| 10 | +++ | A | BB |
| 11 | + | AAA | B |
| 12 | +++ | AAA | BBB |
| 13 | + | AAA | B |
| 14 | + | AAA | B |
| 15 | ++ | AAA | B |
| 16 | +++ | AA | BBB |
| 17 | +++ | AA | BB |
| 18 | +++ | AA | BB |
| 19 | ++ | NA | BBB |
| 27 | +++ | AAA | BB |
| 31 | ++ | AAA | B |
| 32 | + | A | NA |

### Example C

Flow cytometry analysis for EZH2 expression and Western blot analysis for phospho-PP1α and phospho-Hec1

Reagents. MDA-MB-231 (Breast adenocarcinoma, triple negative breast cancer) and SW480 (colorectal adenocarcinoma, stem-like subtype) cells were purchased from ATCC and cultured as per manufacturer's instruction. Thymidine (Sigma, T9250) stock solution (100 mM) was prepared in PBS. 2′-Deoxycytidine (Sigma, D897) stock solution (240 mM) was prepared in distilled H₂O. Nocodazole (Selleck Chemicals, S2775) stock solution (5 mg/mL) was made in dimethyl sulfoxide (DMSO). FxCycle Far Red stain (F10348) for cell cycle analysis was purchased from Thermo Fisher Scientific.

Antibodies. Polyclonal antibodies to phosphorylated-Ser55 Hec1 (PA5-85846; 1:1000) were purchased from ThermoFisher Scientific, and Phospho-Thr320 PP1α (2581S; 1:1000) and Total PP1α (2582S; 1:1000) were purchased from Cell Signaling Technologies. Monoclonal antibodies to Phospho-Ser10 Histone H3 (53348S; 1:1000), Total Histone H3 (14269; 1:5000), and EZH2-FITC (30233, 1:50) antibodies were purchased from Cell Signaling Technologies; Total Hec1 (ab109496; 1:10000) was purchased from Abcam; and Total NEK2 (sc-55601; 1:200) and GAPDH (sc-365062; 1:1000-2000) antibodies were purchased from Santa Cruz Technologies.

Flow Cytometry Analysis. The SW480 cells were treated with NEK2 inhibitors at a concentration ranging from 0-1000 nM for 24 hours. After 24 hours of treatment, cells were detached and dissociated using 0.25% trypsin. After trypsinization, cells were washed with ice-cold PBS and fixed in 4% paraformaldehyde (PFA) at room temperature for 20 minutes. After fixation, cells were permeabilized by incubating in 90% methanol for 30 minutes on ice. Subsequently, cells were stained with FITC conjugated anti-EZH2 antibody (1:50, Cell Signaling technology) for 2 hours at room temperature and FxCycle Far Red stain (Thermo Fisher Scientific) for visualization of EZH2 expression and cell cycle, respectively. Flow cytometry was performed on a Attune NxT Acoustic focusing cytometer (Thermo Fisher Scientific). A minimum of 100,000 events were analyzed in each sample, and the results were evaluated using FlowJo software. Too-small objects, including cell debris and the majority of polyploid cells (DNA content >2N), were excluded from analysis.

Cell Cycle Synchronization. MDA-MB-231 cells were treated with 2 mM thymidine for 16-20 hours then washed with PBS and released into thymidine-free fresh medium containing 24 μM deoxycytidine for 8 hours. After 8 hours, 2 mM thymidine was added to the cells for 16-20 hours to promote cell synchronization at early S-phase. After 16-20-hour incubation, cells were washed with PBS and released from S-phase arrest into fresh thymidine-free medium with 100 ng/ml nocodazole for 8 hours to synchronize cells to mitotic arrest. Effectiveness of NEK2 inhibitors (Cmpd-16 and Cmpd-18) were assessed by adding small molecule inhibitors with nocodozole at 10, 50, 100, and 500 nM concentration at the time of nocodazole addition. After nocodazole or nocodozole+NEK2 inhibitor incubation, cells were harvested for assaying Phospho Hec1, Total Hec1, Phospho PP1a, Total PP1a, Phospho Histone H3, Total Histone H3, NEK2, and GAPDH (as loading control) by immunoblot analysis.

Immunoblot Analysis. Cells were harvested by washing with ice-cold PBS then lysed with ice-cold RIPA (Cell Signaling Technologies, 9806S) buffer containing 1% Halt protease and phosphatase inhibitor cocktail (ThermoFisher Scientific, 78444) on ice. Whole cell lysates were sonicated and centrifuged at 16000×g for 10 minutes at 4° C., and protein concentration of the supernatant were determined using BCA Protein assay kit (ThermoScientific, 23225). Equal amounts of protein lysates (30 μg/lane and 10 μg/lane only for Histone H3 immunoblots) were fractionated on a NuPAGE Novex 4%-12% Bis-Tris Protein Gel (Thermo Fisher Scientific, NP0335BOX) and transferred onto a low fluorescence PVDF membrane (Millipore, IPFL07810). Subsequently, the membranes were blocked with 5% BSA for 1 hour at room temperature and then treated with the appropriate antibody at 4° C. overnight. Goat anti-rabbit IgG conjugated to IRDye700 (926-68171) or 800 (827-08365) and goat anti-mouse IgG conjugated to IRDye 700 (926-68170) or 800 (827-08364) secondary antibodies were used at 1:20000 in 5% BSA in TBST (TBS-Tween 20; Genesee Scientific, 18-235B) were purchased from Li-Cor Biosciences. Protein expression was visualized with Li-Cor Odyssey CLX imaging system. Li-Cor Image Studio software was used for western blot analysis and quantification

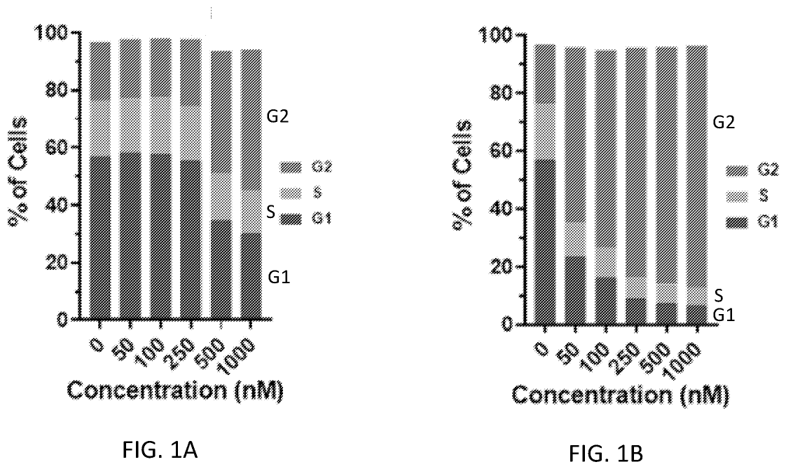

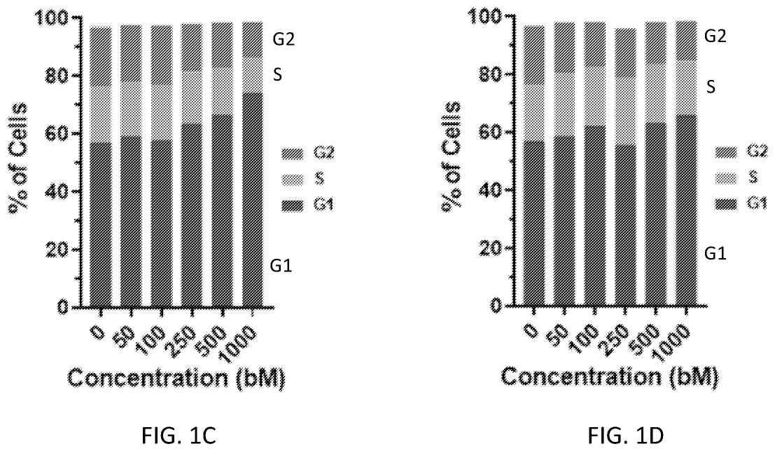

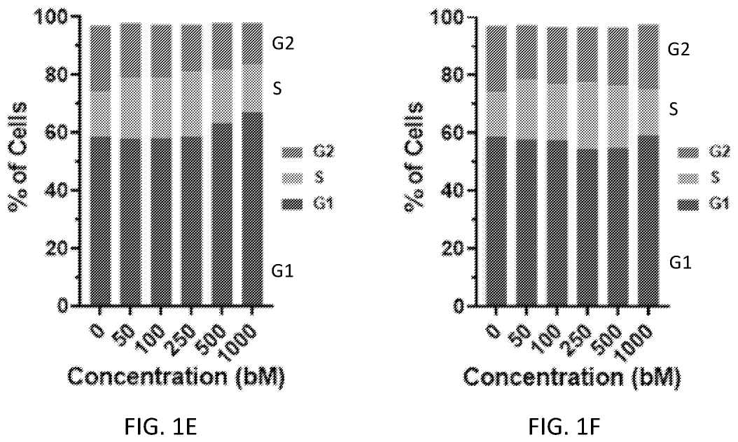

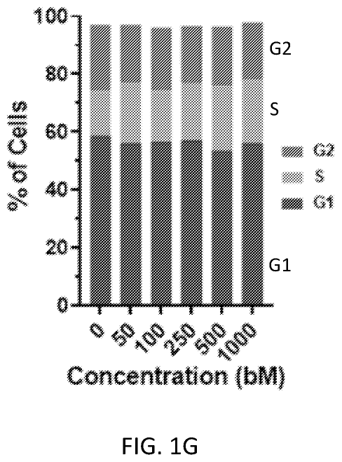

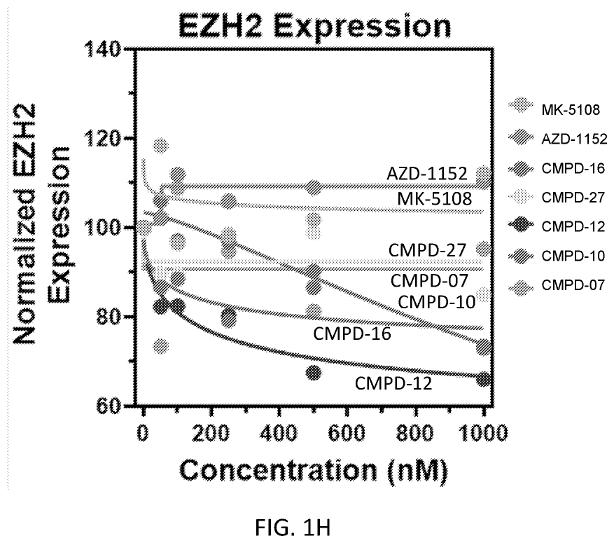

Results. To test the efficacy of NEK2 compounds (Cmpd-16, Cmpd-27, Cmpd-12, Cmpd-10, and Cmpd-07), the ability of the compounds to suppress EZH2 expression was assessed by flow cytometry. Some compounds showing NEK2 activity have shown off-target activity against AURKA/B as well, leading to cell cycle arrest in G2-M phase. To validate the specificity of the NEK2 inhibitors presented herein, cell cycle of the cells after NEK2 inhibition was also queried. AZD1152 (an Astra Zeneca Aurora Kinase B inhibitor) and MK-5108 (a Merck Aurora Kinase A inhibitor) were used as controls. As shown in the cell cycle analysis, MK-5108 and AZD-1152 significantly arrested cells in G2-M phase, while none of the NEK2 inhibitors tested with them affected cell cycle distribution at the concentrations used in the experiment (FIGS. 1A-1G). EZH2 expression analysis in haploid and diploid cells showed dose dependent decrease in EZH2 expression by only Cmpd-16 (IC₅₀ 184.5 nM) and Cmpd-12 (IC₅₀ 213.5 nM) (FIG. 1H). Taken together, these data show that NEK2 inhibition can selectively suppress EZH2 expression without affecting cell cycle distribution.

It is well established that NEK2 plays a critical role in centrosome separation and spindle assembly checkpoint (SAC) during normal cell cycle. Fry, A. M., et al., Front Cell Dev Biol 5 (2017), 102. NEK2 directly phosphorylates PP1α and HEC1 for centrosome separation process and SAC function, respectively. To test efficacy of newly synthesized compounds, phosphorylation of PP1α and HEC1 after treatment with selected NEK2 inhibitors (Cmpd-16 and Cmpd-18) was assessed. Some compounds showing NEK2 inhibitor activity have demonstrated off-target activity against AURK A/B, leading to cell cycle arrest in G2-M phase. To validate the specificity of the NEK2 inhibitors presented herein, phosphorylation of HistoneH3 was also queried as a marker of mitosis.

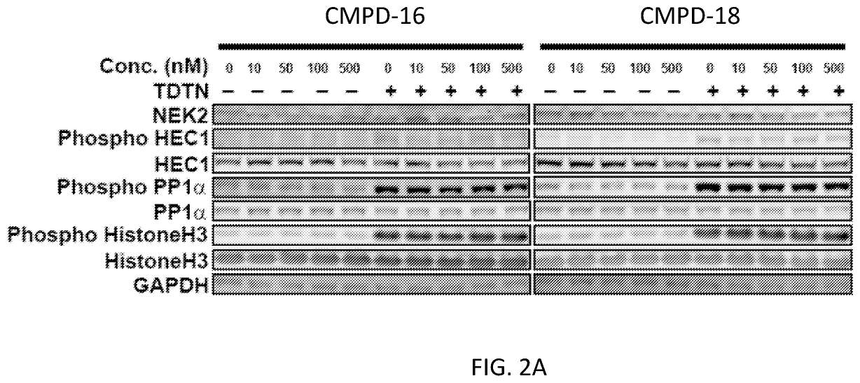

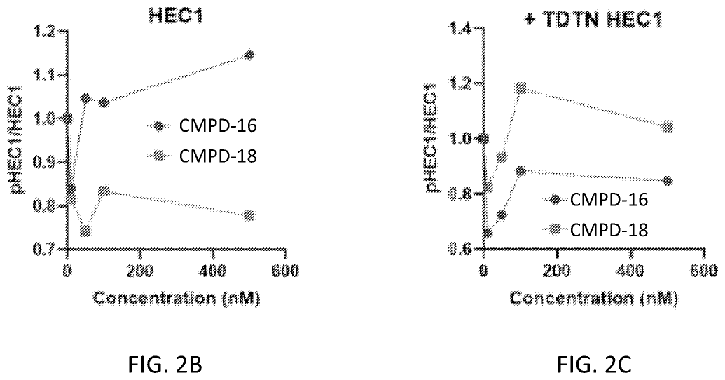

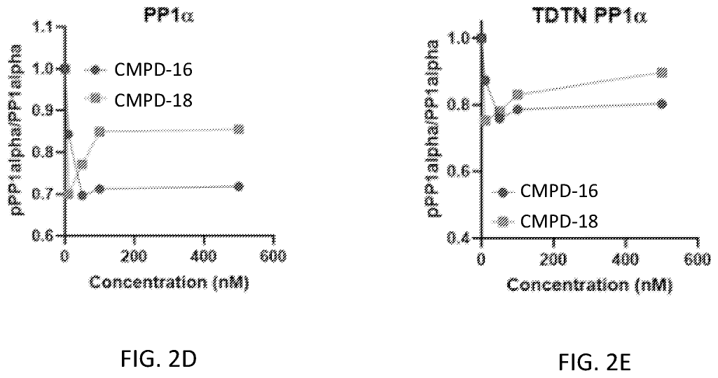

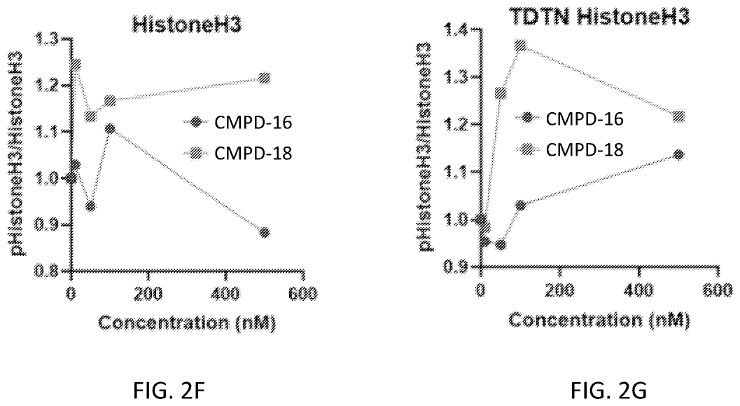

Earlier studies have also demonstrated that NEK2 is a very dynamic protein with very short half-life and its expression peaks at prometaphase onset of cell cycle. Hayes, M. J., et al., Nat Cell Biol 8(2006), 607-614. To see the effect of NEK2 inhibition more clearly, experiments were carried out in cell cycle synchronized cells (TDTN). Western blot analysis demonstrated that cell cycle synchronization with double thymidine block and nocodozole showed increased NEK2 expression in DMSO treated cells (FIG. 2A). It also showed that Cmpd-16 more specifically inhibited phosphorylation of PP1α and HEC1 without interfering in cell cycle progression than Cmpd-18, as demonstrated by phosphorylation of HistoneH3 (FIGS. 2A-2G). Additionally, effect on phosphorylation of HEC1 and PP1α was more pronounced in cell cycle synchronized cells (FIGS. 2A-2G).

In summary, flow cytometry analysis for EZH2 expression and Western blot analysis for phospho-PP1α and phospho-Hec1 in cell cycle synchronized cells demonstrate that Cmpd-16 and Cmpd-12 are specific and potent inhibitors of NEK2 kinase activity.

Various modifications of the invention, in addition to those described herein, will be apparent to those skilled in the art form the foregoing description. Such modifications are also intended to fall within the scope of the appended claims. Each reference cited in the present application, including all patents, applications, and non-patent literature, is incorporated herein by reference in its entirety.

What is claimed is:

1. A compound of Formula I: 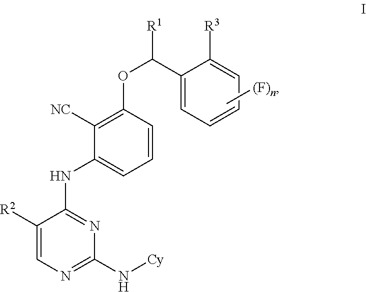
or a pharmaceutically acceptable salt thereof, wherein:
R¹ is H or alkyl;
R² is alkoxy, alkyl, cycloalkyl, halo or —OH;
R³ is methoxy, H or fluoro, and when R³ is H or fluoro, n is 0 or 1, and when R³ is methoxy, n is 0; and
Cy is a moiety having the structure: 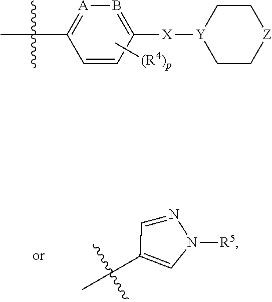
wherein:
A and B are independently selected from CH or N, with the proviso that when one of A or B is N the other is CH;
X is a bond, CH₂, or C(O);
R⁴ is alkoxy or halo;
p is 0, 1 or 2;
Y is N or CH;
Z is NH, N(CH₂)₀₋₂CH₃, N—(CH₂)₁₋₃N(CH₃)₂N(CH₂)₁₋₂OH or O; and
R⁵ is 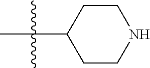 [SMILES: *C1CCNCC1]
or —(CH₂)₁₋₃N(CH₃)₂.

2. The compound of claim 1, wherein R¹ is H or methyl.

3. The compound of claim 2, wherein R¹ is H.

4. The compound of claim 3, wherein R¹ is methyl.

5. The compound of any one of claims 1-4, R² is methoxy, OH, chloro, methyl, ethyl, n-propyl, isopropyl, or cyclopropyl.

6. The compound of claim 5, wherein R² is chloro.

7. The compound of any one of claims 1-6, wherein R³ is fluoro.

8. The compound of any one of claims 1-6, wherein R³ is H and n is 1.

9. The compound of any one of claims 1-8, wherein Cy is:

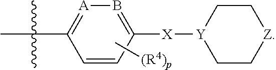

10. The compound of any one of claims 1-9, wherein A and B are each CH.

11. The compound of any one of claims 1-10, wherein R⁴ is methoxy or fluoro.

12. The compound of any one of claims 1-11, wherein p is 0 or 1.

13. The compound of claim 12, wherein p is 0.

14. The compound of any one of claims 1-13, wherein X is a bond.

15. The compound of any one of claims 1-14, wherein Y is N.

16. The compound of any one of claims 1-15, wherein Z is NH, NCH₃ or O.

17. The compound of claim 16, wherein Z is NH.

18. The compound of claim 16, wherein Z is NCH₃.

19. The compound of claim 16, wherein Z is O.

20. The compound of any one of claims 1-15, wherein Z is NCH₂CH₂OH.

21. The compound of any one of claims 1 and 10-20, wherein R¹ is H; R² is chloro; R³ is fluoro and Cy is:

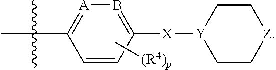

22. The compound of any one of claims 1-21, having the structure of Formula II: 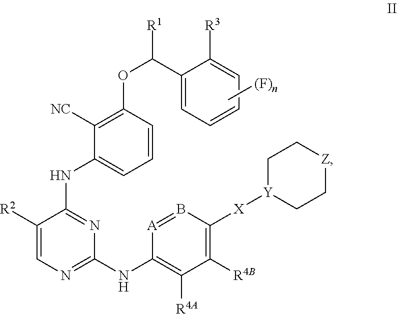
or a pharmaceutically acceptable salt thereof, wherein:
R^{4A} and R^{4B} are each independently selected from H, methoxy or fluoro.

23. The compound of any one of claims 1-8, having the structure of Formula III: 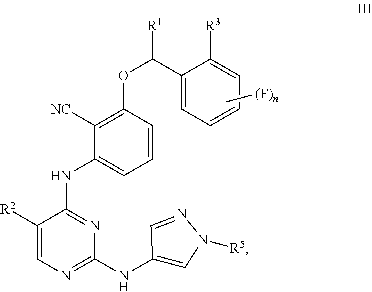
or a pharmaceutically acceptable salt thereof.

24. The compound of any one of claims 1-8 and 23, wherein R⁵ is CH₂CH₂N(CH₃)₂.

25. A compound of claim 1, selected from: 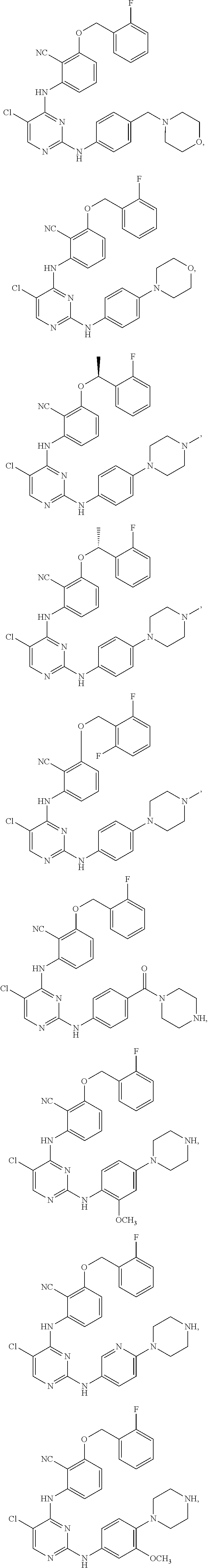 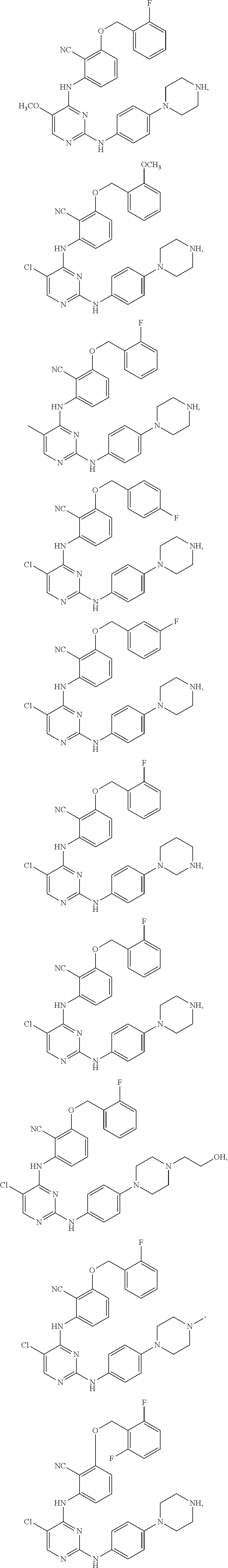 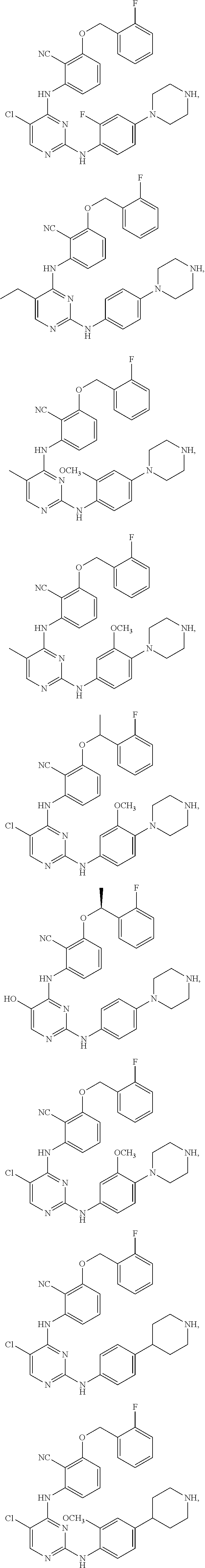 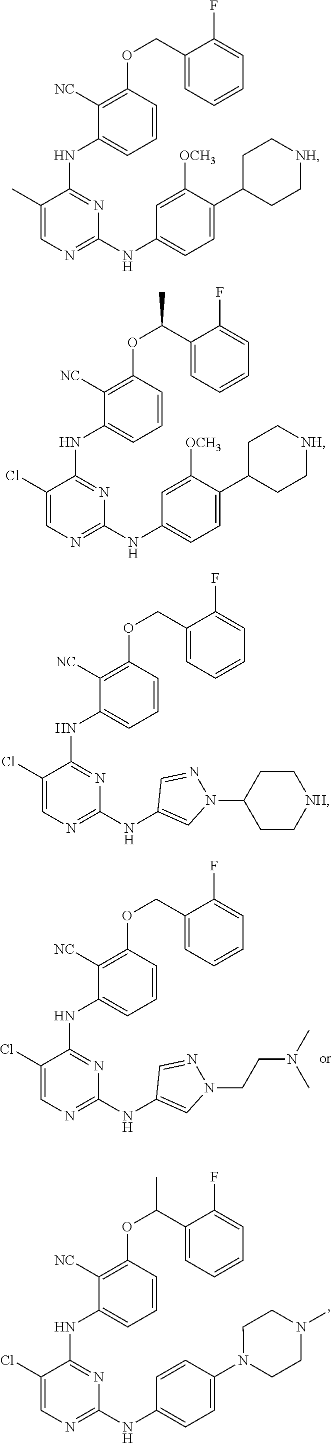
or a pharmaceutically acceptable salt of any of the foregoing.

26. A compound of claim 1, having the following structure: 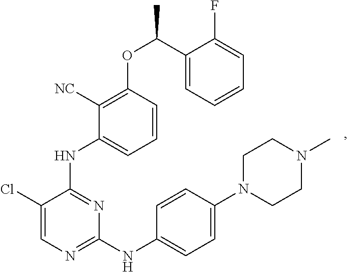 [SMILES: C[C@H](Oc1cccc(Nc2nc(Nc3ccc(N4CCN(C)CC4)cc3)ncc2Cl)c1C#N)c1ccccc1F]
or a pharmaceutically acceptable salt thereof.

27. A compound of claim 1, having the following structure: 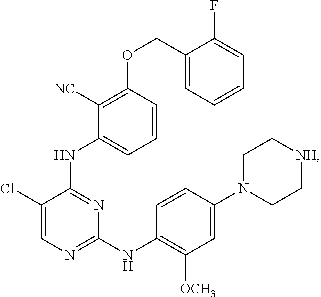 [SMILES: COc1cc(N2CCNCC2)ccc1Nc1ncc(Cl)c(Nc2cccc(OCc3ccccc3F)c2C#N)n1]
or a pharmaceutically acceptable salt thereof.

28. A compound of claim 1, having the following structure: 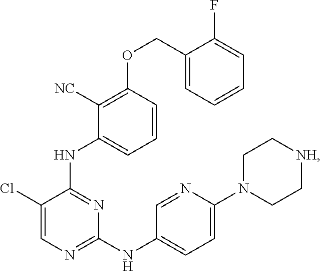 [SMILES: N#Cc1c(Nc2nc(Nc3ccc(N4CCNCC4)nc3)ncc2Cl)cccc1OCc1ccccc1F]
or a pharmaceutically acceptable salt thereof.

29. A compound of claim 1, having the following structure: 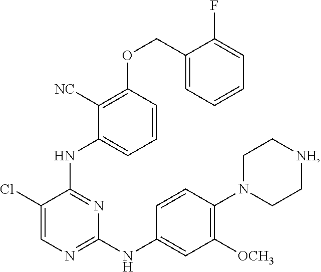 [SMILES: COc1cc(Nc2ncc(Cl)c(Nc3cccc(OCc4ccccc4F)c3C#N)n2)ccc1N1CCNCC1]
or a pharmaceutically acceptable salt thereof.

30. A compound of claim 1, having the following structure: 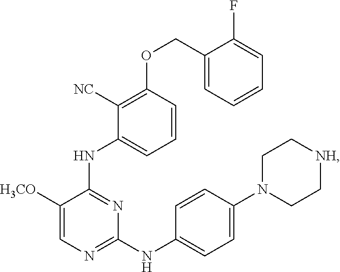 [SMILES: COc1cnc(Nc2ccc(N3CCNCC3)cc2)nc1Nc1cccc(OCc2ccccc2F)c1C#N]
or a pharmaceutically acceptable salt thereof.

31. A compound of claim 1, having the following structure: 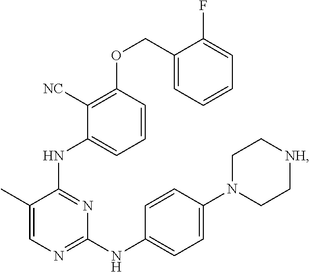 [SMILES: Cc1cnc(Nc2ccc(N3CCNCC3)cc2)nc1Nc1cccc(OCc2ccccc2F)c1C#N]
or a pharmaceutically acceptable salt thereof.

32. A compound of claim 1, having the following structure: 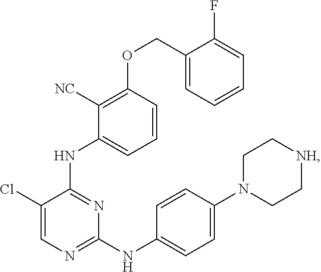 [SMILES: N#Cc1c(Nc2nc(Nc3ccc(N4CCNCC4)cc3)ncc2Cl)cccc1OCc1ccccc1F]
or a pharmaceutically acceptable salt thereof.

33. A compound of claim 1, having the following structure: 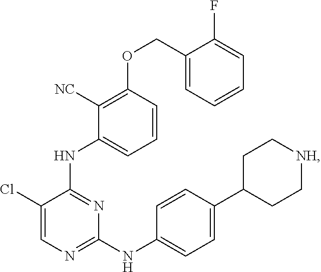 [SMILES: N#Cc1c(Nc2nc(Nc3ccc(C4CCNCC4)cc3)ncc2Cl)cccc1OCc1ccccc1F]
or a pharmaceutically acceptable salt thereof.

34. A compound of claim 1, having the following structure: 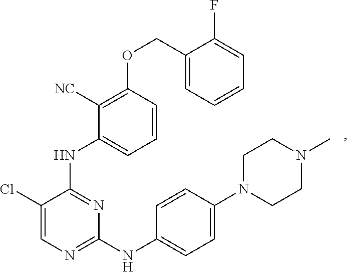 [SMILES: CN1CCN(c2ccc(Nc3ncc(Cl)c(Nc4cccc(OCc5ccccc5F)c4C#N)n3)cc2)CC1]
or a pharmaceutically acceptable salt thereof.

35. A composition comprising at least one compound of any one of claims 1-34, or at least one compound thereof in the form of a pharmaceutically acceptable salt, and at least one pharmaceutically acceptable excipient.

36. A method of inhibiting the activity of NEK2 in a subject having a condition in which NEK2 has activity above normally functioning cells, comprising administering to the subject at least one compound of any one of claims 1-34, or at least one compound thereof in the form of a pharmaceutically acceptable salt, in an amount sufficient to reduce the level of NEK2 activity.

37. A method of treating a disease in a subject, wherein the disease is associated with NEK2 activity, comprising administering to the subject a therapeutically effective amount of at least one compound of any one of claims 1-34, or at least one compound thereof in the form of a pharmaceutically acceptable salt.

38. The method of claim 37, wherein the disease is cancer.

39. A method of treating a cancer in a subject, comprising administering to the subject a therapeutically effective amount of at least one compound of any one of claims 1-34, or at least one compound thereof in the form of a pharmaceutically acceptable salt.

40. The method of claim 38 or 39, wherein the cancer is a solid tumor cancer.

41. The method of claim 40, wherein the cancer is a tumor of the bones, digestive organs, reproductive organs, head, neck, lung, heart, skin, nervous system, endocrine system, neuroendocrine system, urinary system, soft tissue, or brain, melanoma, renal cell cancer, non-small cell lung cancer (NSCLC), colorectal carcinoma (CRC), cervical cancer, ovarian cancer, melanoma, breast carcinoma, neuroendocrine carcinoma, prostate cancer, cholangiocarcinoma, uterine carcinoma, neuroblastoma, peripheral nerve sheath tumor, testicular cancer, bladder cancer, pancreatic cancer and pancreatic cancer.

42. The method of claim 38 or 39, wherein the cancer is a hematologic cancer.

43. The method of claim 42, wherein the hematologic cancer is multiple myeloma, myelodysplastic syndrome (MDS), acute myelogenous leukemia (AML), acute lymphoblastic leukemia (ALL), acute lymphocytic leukemia, chronic lymphogenous leukemia, chronic lymphocytic leukemia (CLL), small lymphocytic lymphoma (SLL), mantle cell lymphoma, diffuse large B-cell lymphoma, follicular lymphoma, or non-Hodgkin's lymphoma.

44. The method of claim 38 or 39, wherein the cancer is breast cancer.

45. The method of claim 38 or 39, wherein the cancer is liver cancer.

46. The method of claim 45, wherein the liver cancer is hepatocellular carcinoma (HCC).

47. The method of claim 38 or 39, wherein the cancer is pancreatic cancer.

48. The method of claim 47, wherein the pancreatic cancer is pancreatic ductal adenocarcinoma.

49. The method of claim 38 or 39, wherein the cancer is colorectal cancer.

50. The method of claim 38 or 39, wherein the cancer is non-small cell lung cancer (NSCLC) or lung adenocarcinoma.

51. The method of claim 38 or 39, wherein the cancer is mantle cell lymphoma or diffuse large B-cell lymphoma.

52. The method of any one of claims 37-51, further comprising administering to the subject one or more additional pharmaceutical agents.

53. A combination comprising at least one compound of any one of claims 1-34, or at least one compound thereof in the form of a pharmaceutically acceptable salt, and one or more additional pharmaceutical agents.

54. A method of inhibiting EZH2 expression or activity in a subject, comprising administering to the subject at least one compound of any one of claims 1-34, or at least one compound thereof in the form of a pharmaceutically acceptable salt, in an amount sufficient to reduce the expression or activity of EZH2.

55. The method of claim 54, wherein the expression or activity of EZH2 is reduced without disrupting cell cycle progression.

56. The method of any one of claims 36-52, 54 and 55, further comprising measuring expression or activity of EZH2 in the subject.

57. The method of any one of claims 36-52, 54 and 55, further comprising measuring expression or activity of EZH2 in the subject, thereby determining efficacy of the compound, or pharmaceutically acceptable salt thereof, in the subject.
